SEQUENCE LISTING (765 sequences)

SEQ ID NO: 1 - DNA, 351 bp, Artificial Sequence
  Synthetic
caggtccagctggtgcagtctgggggaggcttggtacagcctggggggtccctgagactctcctgtgcagcctctggatttactctaagtagttacgacatgcactgggtccgccaatctacaggaaaaggtctggagtgggtctcagctattggttctaccggtgacacatactatccaggctccgtgaagggccgattcaccatcaccagagaaaaagccaagaactccgtgtatcttcaaatgaacagcctgagagccggggacacggctgtgtattactgtgtaagagaggggtgggaggtaccctttgactactggggccagggaaccctggtcactgtctcctca

SEQ ID NO: 2 - protein, 117 aa, Artificial Sequence
  Synthetic
QVQLVQSGGGLVQPGGSLRLSCAASGFTLSSYDMHWVRQSTGKGLEWVSAIGSTGDTYYPGSVKGRFTITREKAKNSVYLQMNSLRAGDTAVYYCVREGWEVPFDYWGQGTLVTVSS

SEQ ID NO: 3 - DNA, 24 bp, Artificial Sequence
  Synthetic
ggatttactctaagtagttacgac

SEQ ID NO: 4 - protein, 8 aa, Artificial Sequence
  Synthetic
GFTLSSYD

SEQ ID NO: 5 - DNA, 21 bp, Artificial Sequence
  Synthetic
attggttctaccggtgacaca

SEQ ID NO: 6 - protein, 7 aa, Artificial Sequence
  Synthetic
IGSTGDT

SEQ ID NO: 7 - DNA, 33 bp, Artificial Sequence
  Synthetic
gtaagagaggggtgggaggtaccctttgactac

SEQ ID NO: 8 - protein, 11 aa, Artificial Sequence
  Synthetic
VREGWEVPFDY

SEQ ID NO: 9 - DNA, 327 bp, Artificial Sequence
  Synthetic
gacatccagatgacccagtctccagccaccctgtctgtgtctccaggggaaagagccgccctctcctgcagggccagtcagagtgttagcagcaacttagcctggtaccaccagaaacctggccaggctcccaggctcctcatctatggtgcatccaccagggccactggtatcccagccaggttcagtggcattgggtctgggacagagttcactctcattatcagcagcctgcagtctgaagattttgcattttatttctgtcagcagtataataactggcctccattcactttcggccctgggaccaaggtggagatcaaacga

SEQ ID NO: 10 - protein, 109 aa, Artificial Sequence
  Synthetic
DIQMTQSPATLSVSPGERAALSCRASQSVSSNLAWYHQKPGQAPRLLIYGASTRATGIPARFSGIGSGTEFTLIISSLQSEDFAFYFCQQYNNWPPFTFGPGTKVEIKR

SEQ ID NO: 11 - DNA, 18 bp, Artificial Sequence
  Synthetic
cagagtgttagcagcaac

SEQ ID NO: 12 - protein, 6 aa, Artificial Sequence
  Synthetic
QSVSSN

SEQ ID NO: 13 - DNA, 9 bp, Artificial Sequence
  Synthetic
ggtgcatcc

SEQ ID NO: 14 - protein, 3 aa, Artificial Sequence
  Synthetic
GAS

SEQ ID NO: 15 - DNA, 30 bp, Artificial Sequence
  Synthetic
cagcagtataataactggcctccattcact

SEQ ID NO: 16 - protein, 10 aa, Artificial Sequence
  Synthetic
QQYNNWPPFT

SEQ ID NO: 17 - DNA, 351 bp, Artificial Sequence
  Synthetic
gaggtgcagctggtggagtctgggggaggcttggtacagcctggggggtccctgagactctcctgtgcagcctctggatttactctaagtagttacgacatgcactgggtccgccaatctacaggaaaaggtctggagtgggtctcagctattggttctaccggtgacacatactatccaggctccgtgaagggccgattcaccatcaccagagaaaaagccaagaactccgtgtatcttcaaatgaacagcctgagagccggggacacggctgtgtattactgtgtaagagaggggtgggaggtaccctttgactactggggccagggaaccctggtcaccgtctcctca

SEQ ID NO: 18 - protein, 117 aa, Artificial Sequence
  Synthetic
EVQLVESGGGLVQPGGSLRLSCAASGFTLSSYDMHWVRQSTGKGLEWVSAIGSTGDTYYPGSVKGRFTITREKAKNSVYLQMNSLRAGDTAVYYCVREGWEVPFDYWGQGTLVTVSS

SEQ ID NO: 19 - DNA, 324 bp, Artificial Sequence
  Synthetic
gaaatagtgatgacgcagtctccagccaccctgtctgtgtctccaggggaaagagccgccctctcctgcagggccagtcagagtgttagcagcaacttagcctggtaccaccagaaacctggccaggctcccaggctcctcatctatggtgcatccaccagggccactggtatcccagccaggttcagtggcattgggtctgggacagagttcactctcattatcagcagcctgcagtctgaagattttgcattttatttctgtcagcagtataataactggcctccattcactttcggccctgggaccaaagtggatatcaaa

SEQ ID NO: 20 - protein, 108 aa, Artificial Sequence
  Synthetic
EIVMTQSPATLSVSPGERAALSCRASQSVSSNLAWYHQKPGQAPRLLIYGASTRATGIPARFSGIGSGTEFTLIISSLQSEDFAFYFCQQYNNWPPFTFGPGTKVDIK

SEQ ID NO: 21 - DNA, 351 bp, Artificial Sequence
  Synthetic
gaggtgcagctggtggagtctgggggaggcttggtacagcctggggggtccctgagactctcctgtgcagcctctggatttactctaagtagttacgacatgcactgggtccgccaagctacaggaaaaggtctggagtgggtctcagctattggttctaccggtgacacatactatccaggctccgtgaagggccgattcaccatctccagagaaaatgccaagaactccttgtatcttcaaatgaacagcctgagagccggggacacggctgtgtattactgtgtaagagaggggtgggaggtaccctttgactactggggccagggaaccctggtcaccgtctcctca

SEQ ID NO: 22 - protein, 117 aa, Artificial Sequence
  Synthetic
EVQLVESGGGLVQPGGSLRLSCAASGFTLSSYDMHWVRQATGKGLEWVSAIGSTGDTYYPGSVKGRFTISRENAKNSLYLQMNSLRAGDTAVYYCVREGWEVPFDYWGQGTLVTVSS

SEQ ID NO: 23 - DNA, 324 bp, Artificial Sequence
  Synthetic
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctccattcactttcggccctgggaccaaagtggatatcaaa

SEQ ID NO: 24 - protein, 108 aa, Artificial Sequence
  Synthetic
EIVMTQSPATLSVSPGERATLSCRASQSVSSNLAWYQQKPGQAPRLLIYGASTRATGIPARFSGSGSGTEFTLTISSLQSEDFAVYYCQQYNNWPPFTFGPGTKVDIK

SEQ ID NO: 25 - DNA, 342 bp, Artificial Sequence
  Synthetic
caggtgcagctggtgcagtctgggggaggcgtggtccagcctgggaggtccctgagactctcctgtgcagcgtctggattcaccttcagtagctatggcatgcactgggtccgccaggctccaggcaaggggctggagtgggtggcgtttataggatttgatggaagtaatatacattatggagactccgtgaggggccgaatcatcatatccagagacaattccgagaacacgttgtatctggaaatgaacagcctgagagccgaggacacggcaatgtactattgtgcgagagagaagggtttagactggggccagggaaccacggtcaccgtctcctca

SEQ ID NO: 26 - protein, 114 aa, Artificial Sequence
  Synthetic
QVQLVQSGGGVVQPGRSLRLSCAASGFTFSSYGMHWVRQAPGKGLEWVAFIGFDGSNIHYGDSVRGRIIISRDNSENTLYLEMNSLRAEDTAMYYCAREKGLDWGQGTTVTVSS

SEQ ID NO: 27 - DNA, 24 bp, Artificial Sequence
  Synthetic
ggattcaccttcagtagctatggc

SEQ ID NO: 28 - protein, 8 aa, Artificial Sequence
  Synthetic
GFTFSSYG

SEQ ID NO: 29 - DNA, 24 bp, Artificial Sequence
  Synthetic
ataggatttgatggaagtaatata

SEQ ID NO: 30 - protein, 8 aa, Artificial Sequence
  Synthetic
IGFDGSNI

SEQ ID NO: 31 - DNA, 21 bp, Artificial Sequence
  Synthetic
gcgagagagaagggtttagac

SEQ ID NO: 32 - protein, 7 aa, Artificial Sequence
  Synthetic
AREKGLD

SEQ ID NO: 33 - DNA, 321 bp, Artificial Sequence
  Synthetic
gccatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattacacttttggccaggggaccaaggtggaaatcaaacga

SEQ ID NO: 34 - protein, 107 aa, Artificial Sequence
  Synthetic
AIQMTQSPSTLSASVGDRVTITCRASQSISSWLAWYQQKPGKAPKLLIYKASSLESGVPSRFSGSGSGTEFTLTISSLQPDDFATYYCQQYNSYYTFGQGTKVEIKR

SEQ ID NO: 35 - DNA, 18 bp, Artificial Sequence
  Synthetic
cagagtattagtagctgg

SEQ ID NO: 36 - protein, 6 aa, Artificial Sequence
  Synthetic
QSISSW

SEQ ID NO: 37 - DNA, 9 bp, Artificial Sequence
  Synthetic
aaggcgtct

SEQ ID NO: 38 - protein, 3 aa, Artificial Sequence
  Synthetic
KAS

SEQ ID NO: 39 - DNA, 24 bp, Artificial Sequence
  Synthetic
caacagtataatagttattacact

SEQ ID NO: 40 - protein, 8 aa, Artificial Sequence
  Synthetic
QQYNSYYT

SEQ ID NO: 41 - DNA, 342 bp, Artificial Sequence
  Synthetic
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcgtttataggatttgatggaagtaatatacattatggagactccgtgaggggccgaatcatcatatccagagacaattccgagaacacgttgtatctggaaatgaacagcctgagagccgaggacacggcaatgtactattgtgcgagagagaagggtttagactggggccagggaaccctggtcaccgtctcctca

SEQ ID NO: 42 - protein, 114 aa, Artificial Sequence
  Synthetic
QVQLVESGGGVVQPGRSLRLSCAASGFTFSSYGMHWVRQAPGKGLEWVAFIGFDGSNIHYGDSVRGRIIISRDNSENTLYLEMNSLRAEDTAMYYCAREKGLDWGQGTLVTVSS

SEQ ID NO: 43 - DNA, 318 bp, Artificial Sequence
  Synthetic
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattacacttttggccaggggaccaagctggagatcaaa

SEQ ID NO: 44 - protein, 106 aa, Artificial Sequence
  Synthetic
DIQMTQSPSTLSASVGDRVTITCRASQSISSWLAWYQQKPGKAPKLLIYKASSLESGVPSRFSGSGSGTEFTLTISSLQPDDFATYYCQQYNSYYTFGQGTKLEIK

SEQ ID NO: 45 - DNA, 342 bp, Artificial Sequence
  Synthetic
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttataggatttgatggaagtaatatatactatgcagactccgtgaagggccgattcaccatctccagagacaattccaagaacacgctgtatctgcaaatgaacagcctgagagctgaggacacggctgtgtattactgtgcgagagagaagggtttagactggggccagggaaccctggtcaccgtctcctca

SEQ ID NO: 46 - protein, 114 aa, Artificial Sequence
  Synthetic
QVQLVESGGGVVQPGRSLRLSCAASGFTFSSYGMHWVRQAPGKGLEWVAVIGFDGSNIYYADSVKGRFTISRDNSKNTLYLQMNSLRAEDTAVYYCAREKGLDWGQGTLVTVSS

SEQ ID NO: 47 - DNA, 319 bp, Artificial Sequence
  Synthetic
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattacacttttggccaggggaccaagctggagatcaaac

SEQ ID NO: 48 - protein, 106 aa, Artificial Sequence; identical to SEQ ID NO: 44
  Synthetic

SEQ ID NO: 49 - DNA, 342 bp, Artificial Sequence
  Synthetic
caggtgcagctgcaggagtctgggggaggcgtggtccagcctgggaggtccctgagactctcctgtgcagcgtctggattcaccttcagtagctatggcatgcactgggtccgccaggctccaggcaaggggctggagtgggtggcgtttataggatttgatggaagtaatatatattatggagactccgtgaggggccgaatcatcatatccagagacaattccgagaacacgttgtatctggaaatgaacagcctgagagccgaggacacggcagtgtattattgtgcgagagagaagggtttagactggggccagggaaccctggtcactgtctcctca

SEQ ID NO: 50 - protein, 114 aa, Artificial Sequence
  Synthetic
QVQLQESGGGVVQPGRSLRLSCAASGFTFSSYGMHWVRQAPGKGLEWVAFIGFDGSNIYYGDSVRGRIIISRDNSENTLYLEMNSLRAEDTAVYYCAREKGLDWGQGTLVTVSS

SEQ ID NO: 51 - DNA, 24 bp, Artificial Sequence
  Synthetic
ggattcaccttcagtagctatggc

SEQ ID NO: 52 - protein, 8 aa, Artificial Sequence
  Synthetic
GFTFSSYG

SEQ ID NO: 53 - DNA, 24 bp, Artificial Sequence
  Synthetic
ataggatttgatggaagtaatata

SEQ ID NO: 54 - protein, 8 aa, Artificial Sequence
  Synthetic
IGFDGSNI

SEQ ID NO: 55 - DNA, 21 bp, Artificial Sequence
  Synthetic
gcgagagagaagggtttagac

SEQ ID NO: 56 - protein, 7 aa, Artificial Sequence
  Synthetic
AREKGLD

SEQ ID NO: 57 - DNA, 342 bp, Artificial Sequence
  Synthetic
gccatccagatgacccagtctccagactccctggctgtgtctctgggcgagagggccaccatcaactgcaagtccagccagagtgtttttcacacctccaacaataagaactacttagtttggtatcagcagaaaccaggacagcctcctaagttgctcctttactgggcctctacccgggaatccggggtccctgaccgattcagtggcagcgggtctgggacagatttcactctcaccatcagcagcctgcaggctgaagatgtggcaaattattactgtcaccaatattacagtattccgtggacgttcggccaagggaccaaggtggagatcaaacga

SEQ ID NO: 58 - protein, 114 aa, Artificial Sequence
  Synthetic
AIQMTQSPDSLAVSLGERATINCKSSQSVFHTSNNKNYLVWYQQKPGQPPKLLLYWASTRESGVPDRFSGSGSGTDFTLTISSLQAEDVANYYCHQYYSIPWTFGQGTKVEIKR

SEQ ID NO: 59 - DNA, 36 bp, Artificial Sequence
  Synthetic
cagagtgtttttcacacctccaacaataagaactac

SEQ ID NO: 60 - protein, 12 aa, Artificial Sequence
  Synthetic
QSVFHTSNNKNY

SEQ ID NO: 61 - DNA, 9 bp, Artificial Sequence
  Synthetic
tgggcctct

SEQ ID NO: 62 - protein, 3 aa, Artificial Sequence
  Synthetic
WAS

SEQ ID NO: 63 - DNA, 27 bp, Artificial Sequence
  Synthetic
caccaatattacagtattccgtggacg

SEQ ID NO: 64 - protein, 9 aa, Artificial Sequence
  Synthetic
HQYYSIPWT

SEQ ID NO: 65 - DNA, 342 bp, Artificial Sequence
  Synthetic
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcgtttataggatttgatggaagtaatatatattatggagactccgtgaggggccgaatcatcatatccagagacaattccgagaacacgttgtatctggaaatgaacagcctgagagccgaggacacggcagtgtattattgtgcgagagagaagggtttagactggggccagggaaccctggtcaccgtctcctca

SEQ ID NO: 66 - protein, 114 aa, Artificial Sequence
  Synthetic
QVQLVESGGGVVQPGRSLRLSCAASGFTFSSYGMHWVRQAPGKGLEWVAFIGFDGSNIYYGDSVRGRIIISRDNSENTLYLEMNSLRAEDTAVYYCAREKGLDWGQGTLVTVSS

SEQ ID NO: 67 - DNA, 339 bp, Artificial Sequence
  Synthetic
gacatcgtgatgacccagtctccagactccctggctgtgtctctgggcgagagggccaccatcaactgcaagtccagccagagtgtttttcacacctccaacaataagaactacttagtttggtatcagcagaaaccaggacagcctcctaagttgctcctttactgggcctctacccgggaatccggggtccctgaccgattcagtggcagcgggtctgggacagatttcactctcaccatcagcagcctgcaggctgaagatgtggcaaattattactgtcaccaatattacagtattccgtggacgttcggccaagggaccaaggtggaaatcaaa

SEQ ID NO: 68 - protein, 113 aa, Artificial Sequence
  Synthetic
DIVMTQSPDSLAVSLGERATINCKSSQSVFHTSNNKNYLVWYQQKPGQPPKLLLYWASTRESGVPDRFSGSGSGTDFTLTISSLQAEDVANYYCHQYYSIPWTFGQGTKVEIK

SEQ ID NO: 69 - DNA, 342 bp, Artificial Sequence; identical to SEQ ID NO: 45
  Synthetic

SEQ ID NO: 70 - protein, 114 aa, Artificial Sequence; identical to SEQ ID NO: 46
  Synthetic

SEQ ID NO: 71 - DNA, 339 bp, Artificial Sequence
  Synthetic
gacatcgtgatgacccagtctccagactccctggctgtgtctctgggcgagagggccaccatcaactgcaagtccagccagagtgtttttcacacctccaacaataagaactacttagcttggtaccagcagaaaccaggacagcctcctaagctgctcatttactgggcctctacccgggaatccggggtccctgaccgattcagtggcagcgggtctgggacagatttcactctcaccatcagcagcctgcaggctgaagatgtggcagtttattactgtcaccaatattacagtattccgtggacgttcggccaagggaccaaggtggaaatcaaa

SEQ ID NO: 72 - protein, 113 aa, Artificial Sequence
  Synthetic
DIVMTQSPDSLAVSLGERATINCKSSQSVFHTSNNKNYLAWYQQKPGQPPKLLIYWASTRESGVPDRFSGSGSGTDFTLTISSLQAEDVAVYYCHQYYSIPWTFGQGTKVEIK

SEQ ID NO: 73 - DNA, 354 bp, Artificial Sequence
  Synthetic
gaagtgcagctggtgcagtctgggggaggcttggtacagcctggggggtccctgagactctcctgtgcagcctctggattcacctttaacaactatgccatgaactgggtccgccaggctccaggaaagggactggactgggtctcaactattagtggtagcggtggtactacaaactacgcagactccgtgaagggccgtttcattatttcccgagacagttccaaacacacgctgtatctgcaaatgaacagcctgagagccgaggacacggccgtatattactgtgcgaaagattctaactggggaaatttcgatctctggggccgtggcaccacggtcactgtctcctca

SEQ ID NO: 74 - protein, 118 aa, Artificial Sequence
  Synthetic
EVQLVQSGGGLVQPGGSLRLSCAASGFTFNNYAMNWVRQAPGKGLDWVSTISGSGGTTNYADSVKGRFIISRDSSKHTLYLQMNSLRAEDTAVYYCAKDSNWGNFDLWGRGTTVTVSS

SEQ ID NO: 75 - DNA, 24 bp, Artificial Sequence
  Synthetic
ggattcacctttaacaactatgcc

SEQ ID NO: 76 - protein, 8 aa, Artificial Sequence
  Synthetic
GFTFNNYA

SEQ ID NO: 77 - DNA, 24 bp, Artificial Sequence
  Synthetic
attagtggtagcggtggtactaca

SEQ ID NO: 78 - protein, 8 aa, Artificial Sequence
  Synthetic
ISGSGGTT

SEQ ID NO: 79 - DNA, 33 bp, Artificial Sequence
  Synthetic
gcgaaagattctaactggggaaatttcgatctc

SEQ ID NO: 80 - protein, 11 aa, Artificial Sequence
  Synthetic
AKDSNWGNFDL

SEQ ID NO: 81 - DNA, 342 bp, Artificial Sequence
  Synthetic
gacatccagatgacccagtctccagactccctggctgtgtctctgggcgagagggccaccatcaactgcaagtccagccagagtgttttatacaggtccaacaataggaacttcttaggttggtaccagcagaaaccagggcagcctcctaatctactcatttactgggcatctacccgggaatccggggtccctgaccgattcagtggcagcgggtctgggacagatttcactctcaccatcagcagcctgcaggctgaagatgtggcagtttattactgtcaacaatattatactactccgtacacttttggccaggggaccaaggtggaaatcaaacga

SEQ ID NO: 82 - protein, 114 aa, Artificial Sequence
  Synthetic
DIQMTQSPDSLAVSLGERATINCKSSQSVLYRSNNRNFLGWYQQKPGQPPNLLIYWASTRESGVPDRFSGSGSGTDFTLTISSLQAEDVAVYYCQQYYTTPYTFGQGTKVEIKR

SEQ ID NO: 83 - DNA, 36 bp, Artificial Sequence
  Synthetic
cagagtgttttatacaggtccaacaataggaacttc

SEQ ID NO: 84 - protein, 12 aa, Artificial Sequence
  Synthetic
QSVLYRSNNRNF

SEQ ID NO: 85 - DNA, 9 bp, Artificial Sequence
  Synthetic
tgggcatct

SEQ ID NO: 86 - protein, 3 aa, Artificial Sequence
  Synthetic
WAS

SEQ ID NO: 87 - DNA, 27 bp, Artificial Sequence
  Synthetic
caacaatattatactactccgtacact

SEQ ID NO: 88 - protein, 9 aa, Artificial Sequence
  Synthetic
QQYYTTPYT

SEQ ID NO: 89 - DNA, 354 bp, Artificial Sequence
  Synthetic
gaggtgcagctggtggagtctgggggaggcttggtacagcctggggggtccctgagactctcctgtgcagcctctggattcacctttaacaactatgccatgaactgggtccgccaggctccaggaaagggactggactgggtctcaactattagtggtagcggtggtactacaaactacgcagactccgtgaagggccgtttcattatttcccgagacagttccaaacacacgctgtatctgcaaatgaacagcctgagagccgaggacacggccgtatattactgtgcgaaagattctaactggggaaatttcgatctctggggccgtggcaccctggtcactgtctcctca

SEQ ID NO: 90 - protein, 118 aa, Artificial Sequence [sequence omitted]

SEQ ID NO: 91 - DNA, 339 bp, Artificial Sequence
  Synthetic
gacatcgtgatgacccagtctccagactccctggctgtgtctctgggcgagagggccaccatcaactgcaagtccagccagagtgttttatacaggtccaacaataggaacttcttaggttggtaccagcagaaaccagggcagcctcctaatctactcatttactgggcatctacccgggaatccggggtccctgaccgattcagtggcagcgggtctgggacagatttcactctcaccatcagcagcctgcaggctgaagatgtggcagtttattactgtcaacaatattatactactccgtacacttttggccaggggaccaagctggagatcaaa

SEQ ID NO: 92 - protein, 113 aa, Artificial Sequence [sequence omitted]

SEQ ID NO: 93 - DNA, 354 bp, Artificial Sequence
  Synthetic
gaggtgcagctggtggagtctgggggaggcttggtacagcctggggggtccctgagactctcctgtgcagcctctggattcacctttaacaactatgccatgagctgggtccgccaggctccagggaaggggctggagtgggtctcagctattagtggtagcggtggtactacatactacgcagactccgtgaagggccggttcaccatctccagagacaattccaagaacacgctgtatctgcaaatgaacagcctgagagccgaggacacggccgtatattactgtgcgaaagattctaactggggaaatttcgatctctggggccgtggcaccctggtcactgtctcctca

SEQ ID NO: 94 - protein, 118 aa, Artificial Sequence
  Synthetic
EVQLVESGGGLVQPGGSLRLSCAASGFTFNNYAMSWVRQAPGKGLEWVSAISGSGGTTYYADSVKGRFTISRDNSKNTLYLQMNSLRAEDTAVYYCAKDSNWGNFDLWGRGTLVTVSS

SEQ ID NO: 95 - DNA, 339 bp, Artificial Sequence
  Synthetic
gacatcgtgatgacccagtctccagactccctggctgtgtctctgggcgagagggccaccatcaactgcaagtccagccagagtgttttatacaggtccaacaataggaacttcttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcaacaatattatactactccgtacacttttggccaggggaccaagctggagatcaaa

SEQ ID NO: 96 - protein, 113 aa, Artificial Sequence
  Synthetic
DIVMTQSPDSLAVSLGERATINCKSSQSVLYRSNNRNFLAWYQQKPGQPPKLLIYWASTRESGVPDRFSGSGSGTDFTLTISSLQAEDVAVYYCQQYYTTPYTFGQGTKLEIK

SEQ ID NO: 97 - DNA, 351 bp, Artificial Sequence
  Synthetic
caggtgcagctggtgcagtctgggggaggcttggtacagcctggggggtccctgagactctcctgtgcagtctctggattcaccctcagtagctacgatatgcactgggtccgccaacctacaggaaaaggtctggagtgggtctcagctattggttctactggtgacacatactatccaggctccgtgaagggccgattcaccatctccagagaaaatgccaagaactccttgtatcttcaaatgaacagcctgagagccggggacacggctgtgtattactgtgcaagagagggatgggacgtaccctttgacttctggggccagggaaccctggtcaccgtctcctca

SEQ ID NO: 98 - protein, 117 aa, Artificial Sequence
  Synthetic
QVQLVQSGGGLVQPGGSLRLSCAVSGFTLSSYDMHWVRQPTGKGLEWVSAIGSTGDTYYPGSVKGRFTISRENAKNSLYLQMNSLRAGDTAVYYCAREGWDVPFDFWGQGTLVTVSS

SEQ ID NO: 99 - DNA, 24 bp, Artificial Sequence
  Synthetic
ggattcaccctcagtagctacgat

SEQ ID NO: 100 - protein, 8 aa, Artificial Sequence
  Synthetic
GFTLSSYD

SEQ ID NO: 101 - DNA, 21 bp, Artificial Sequence
  Synthetic
attggttctactggtgacaca

SEQ ID NO: 102 - protein, 7 aa, Artificial Sequence
  Synthetic
IGSTGDT

SEQ ID NO: 103 - DNA, 33 bp, Artificial Sequence
  Synthetic
gcaagagagggatgggacgtaccctttgacttc

SEQ ID NO: 104 - protein, 11 aa, Artificial Sequence
  Synthetic
AREGWDVPFDF

SEQ ID NO: 105 - DNA, 324 bp, Artificial Sequence
  Synthetic
gccatccagttgacccagtctccatcctccctgtctgcatctgtaggagacagagtcaccatcacttgccgggcaagtcaggacattagaaatgatttaggctggtatcagcagaaaccagggaaagcccctaagctcctgatctatgctgcatccagtttacaaagtggggtcccatcacggttcagcggcagtggatctggcacagatttcactctcaccatcagcagcctgcagcctgaagattttgcaacttattactgtctacaagattacaattacccgtggacgttcggccaagggaccaaggtggagatcaaacga

SEQ ID NO: 106 - protein, 108 aa, Artificial Sequence
  Synthetic
AIQLTQSPSSLSASVGDRVTITCRASQDIRNDLGWYQQKPGKAPKLLIYAASSLQSGVPSRFSGSGSGTDFTLTISSLQPEDFATYYCLQDYNYPWTFGQGTKVEIKR

SEQ ID NO: 107 - DNA, 18 bp, Artificial Sequence
  Synthetic
caggacattagaaatgat

SEQ ID NO: 108 - protein, 6 aa, Artificial Sequence
  Synthetic
QDIRND

SEQ ID NO: 109 - DNA, 9 bp, Artificial Sequence
  Synthetic
gctgcatcc

SEQ ID NO: 110 - protein, 3 aa, Artificial Sequence
  Synthetic
AAS

SEQ ID NO: 111 - DNA, 27 bp, Artificial Sequence
  Synthetic
ctacaagattacaattacccgtggacg

SEQ ID NO: 112 - protein, 9 aa, Artificial Sequence
  Synthetic
LQDYNYPWT

SEQ ID NO: 113 - DNA, 351 bp, Artificial Sequence
  Synthetic
gaggtgcagctggtggagtctgggggaggcttggtacagcctggggggtccctgagactctcctgtgcagtctctggattcaccctcagtagctacgatatgcactgggtccgccaacctacaggaaaaggtctggagtgggtctcagctattggttctactggtgacacatactatccaggctccgtgaagggccgattcaccatctccagagaaaatgccaagaactccttgtatcttcaaatgaacagcctgagagccggggacacggctgtgtattactgtgcaagagagggatgggacgtaccctttgacttctggggccagggaaccctggtcaccgtctcctca

SEQ ID NO: 114 - protein, 117 aa, Artificial Sequence
  Synthetic
EVQLVESGGGLVQPGGSLRLSCAVSGFTLSSYDMHWVRQPTGKGLEWVSAIGSTGDTYYPGSVKGRFTISRENAKNSLYLQMNSLRAGDTAVYYCAREGWDVPFDFWGQGTLVTVSS

SEQ ID NO: 115 - DNA, 321 bp, Artificial Sequence
  Synthetic
gccatccagatgacccagtctccatcctccctgtctgcatctgtaggagacagagtcaccatcacttgccgggcaagtcaggacattagaaatgatttaggctggtatcagcagaaaccagggaaagcccctaagctcctgatctatgctgcatccagtttacaaagtggggtcccatcacggttcagcggcagtggatctggcacagatttcactctcaccatcagcagcctgcagcctgaagattttgcaacttattactgtctacaagattacaattacccgtggacgttcggccaagggaccaaggtggaaatcaaa

SEQ ID NO: 116 - protein, 107 aa, Artificial Sequence
  Synthetic
AIQMTQSPSSLSASVGDRVTITCRASQDIRNDLGWYQQKPGKAPKLLIYAASSLQSGVPSRFSGSGSGTDFTLTISSLQPEDFATYYCLQDYNYPWTFGQGTKVEIK

SEQ ID NO: 117 - DNA, 351 bp, Artificial Sequence
  Synthetic
gaggtgcagctggtggagtctgggggaggcttggtacagcctggggggtccctgagactctcctgtgcagcctctggattcaccctcagtagctacgatatgcactgggtccgccaagctacaggaaaaggtctggagtgggtctcagctattggttctactggtgacacatactatccaggctccgtgaagggccgattcaccatctccagagaaaatgccaagaactccttgtatcttcaaatgaacagcctgagagccggggacacggctgtgtattactgtgcaagagagggatgggacgtaccctttgacttctggggccagggaaccctggtcaccgtctcctca

SEQ ID NO: 118 - protein, 117 aa, Artificial Sequence
  Synthetic
EVQLVESGGGLVQPGGSLRLSCAASGFTLSSYDMHWVRQATGKGLEWVSAIGSTGDTYYPGSVKGRFTISRENAKNSLYLQMNSLRAGDTAVYYCAREGWDVPFDFWGQGTLVTVSS

SEQ ID NO: 119 - DNA, 321 bp, Artificial Sequence
  Synthetic
gccatccagatgacccagtctccatcctccctgtctgcatctgtaggagacagagtcaccatcacttgccgggcaagtcaggacattagaaatgatttaggctggtatcagcagaaaccagggaaagcccctaagctcctgatctatgctgcatccagtttacaaagtggggtcccatcaaggttcagcggcagtggatctggcacagatttcactctcaccatcagcagcctgcagcctgaagattttgcaacttattactgtctacaagattacaattacccgtggacgttcggccaagggaccaaggtggaaatcaaa

SEQ ID NO: 120 - protein, 107 aa, Artificial Sequence; identical to SEQ ID NO: 116
  Synthetic

SEQ ID NO: 121 - DNA, 384 bp, Artificial Sequence
  Synthetic
caggtgcagctgcaggagtcggggccaggactggtgaagccttcggagaccctgtccctcacctgcactgtctctggggactccatcaatacttactactggagctggttccggcagcccccagggaagggactggagtggattgggtatatctattatagtggaaccaccaactacaacccctccctcaagagtcgagtcaccatatcaatagacacgcccaggaaccagttctccctgaagctgatctctgtgaccgcagcggacacggccgtgtattactgtgcgagagagaggattactatgattcggggagttaccctctactattactcctacggtatggacgtctggggccaagggaccacggtcaccgtctcctca

SEQ ID NO: 122 - protein, 128 aa, Artificial Sequence
  Synthetic
QVQLQESGPGLVKPSETLSLTCTVSGDSINTYYWSWFRQPPGKGLEWIGYIYYSGTTNYNPSLKSRVTISIDTPRNQFSLKLISVTAADTAVYYCARERITMIRGVTLYYYSYGMDVWGQGTTVTVSS

SEQ ID NO: 123 - DNA, 24 bp, Artificial Sequence
  Synthetic
ggggactccatcaatacttactac

SEQ ID NO: 124 - protein, 8 aa, Artificial Sequence
  Synthetic
GDSINTYY

SEQ ID NO: 125 - DNA, 21 bp, Artificial Sequence
  Synthetic
atctattatagtggaaccacc

SEQ ID NO: 126 - protein, 7 aa, Artificial Sequence
  Synthetic
IYYSGTT

SEQ ID NO: 127 - DNA, 66 bp, Artificial Sequence
  Synthetic
gcgagagagaggattactatgattcggggagttaccctctactattactcctacggtatggacgtc

SEQ ID NO: 128 - protein, 22 aa, Artificial Sequence
  Synthetic
ARERITMIRGVTLYYYSYGMDV

SEQ ID NO: 129 - DNA, 324 bp, Artificial Sequence
  Synthetic
gacatccagatgacccagtctccatccttcctgtctgcatctgtaggagacagagtcaccatcacttgctgggccagtcaggacattagcagttatttagcctggtatcagcaaaaaccagggatagcccctaagctcctgatctatgctgcatccactttgcaaagtggggtcccatcaaggttcggcggcagtggatctgggacagaattcactctcacaatcagcagcctgcagcctgaagattttgcaacttattactgtcaacagcttaatagttaccctcggacgttcggccaagggaccaaggtggaaatcaaacga

SEQ ID NO: 130 - protein, 108 aa, Artificial Sequence
  Synthetic
DIQMTQSPSFLSASVGDRVTITCWASQDISSYLAWYQQKPGIAPKLLIYAASTLQSGVPSRFGGSGSGTEFTLTISSLQPEDFATYYCQQLNSYPRTFGQGTKVEIKR

SEQ ID NO: 131 - DNA, 18 bp, Artificial Sequence
  Synthetic
caggacattagcagttat

SEQ ID NO: 132 - protein, 6 aa, Artificial Sequence
  Synthetic
QDISSY

SEQ ID NO: 133 - DNA, 9 bp, Artificial Sequence
  Synthetic
gctgcatcc

SEQ ID NO: 134 - protein, 3 aa, Artificial Sequence
  Synthetic
AAS

SEQ ID NO: 135 - DNA, 27 bp, Artificial Sequence
  Synthetic
caacagcttaatagttaccctcggacg

SEQ ID NO: 136 - protein, 9 aa, Artificial Sequence
  Synthetic
QQLNSYPRT

SEQ ID NO: 137 - DNA, 384 bp, Artificial Sequence; identical to SEQ ID NO: 121
  Synthetic

SEQ ID NO: 138 - protein, 128 aa, Artificial Sequence; identical to SEQ ID NO: 122
  Synthetic

SEQ ID NO: 139 - DNA, 321 bp, Artificial Sequence
  Synthetic
gacatccagatgacccagtctccatccttcctgtctgcatctgtaggagacagagtcaccatcacttgctgggccagtcaggacattagcagttatttagcctggtatcagcaaaaaccagggatagcccctaagctcctgatctatgctgcatccactttgcaaagtggggtcccatcaaggttcggcggcagtggatctgggacagaattcactctcacaatcagcagcctgcagcctgaagattttgcaacttattactgtcaacagcttaatagttaccctcggacgttcggccaagggaccaaggtggaaatcaaa

SEQ ID NO: 140 - protein, 107 aa, Artificial Sequence
  Synthetic
DIQMTQSPSFLSASVGDRVTITCWASQDISSYLAWYQQKPGIAPKLLIYAASTLQSGVPSRFGGSGSGTEFTLTISSLQPEDFATYYCQQLNSYPRTFGQGTKVEIK

SEQ ID NO: 141 - DNA, 384 bp, Artificial Sequence
  Synthetic
caggtgcagctgcaggagtcgggcccaggactggtgaagccttcggagaccctgtccctcacctgcactgtctctggggactccatcaatacttactactggagctggatccggcagcccccagggaagggactggagtggattgggtatatctattatagtggaaccaccaactacaacccctccctcaagagtcgagtcaccatatcagtagacacgtccaagaaccagttctccctgaagctgagctctgtgaccgctgcggacacggccgtgtattactgtgcgagagagaggattactatgattcggggagttaccctctactattactcctacggtatggacgtctggggccaagggaccacggtcaccgtctcctca

SEQ ID NO: 142 - protein, 128 aa, Artificial Sequence
  Synthetic
QVQLQESGPGLVKPSETLSLTCTVSGDSINTYYWSWIRQPPGKGLEWIGYIYYSGTTNYNPSLKSRVTISVDTSKNQFSLKLSSVTAADTAVYYCARERITMIRGVTLYYYSYGMDVWGQGTTVTVSS

SEQ ID NO: 143 - DNA, 321 bp, Artificial Sequence
  Synthetic
gacatccagatgacccagtctccatcctccctgtctgcatctgtaggagacagagtcaccatcacttgccgggcaagtcaggacattagcagttatttaggctggtatcagcagaaaccagggaaagcccctaagcgcctgatctatgctgcatccagtttgcaaagtggggtcccatcaaggttcagcggcagtggatctgggacagaattcactctcacaatcagcagcctgcagcctgaagattttgcaacttattactgtcaacagcttaatagttaccctcggacgttcggccaagggaccaaggtggaaatcaaa

SEQ ID NO: 144 - protein, 107 aa, Artificial Sequence
  Synthetic
DIQMTQSPSSLSASVGDRVTITCRASQDISSYLGWYQQKPGKAPKRLIYAASSLQSGVPSRFSGSGSGTEFTLTISSLQPEDFATYYCQQLNSYPRTFGQGTKVEIK

SEQ ID NO: 145 - DNA, 378 bp, Artificial Sequence
  Synthetic
caggtgcagctggtgcagtctggagctgaggtgaagaagcctggggcctcagtgaaggtctcctgcaaggcttctggttacacctttaccaactatggtatcagctgggtgcgacaggcccctggacaaggacttgagttaatgggatggattagtggttacaatggtaacacaaactatgcacaagaactccaggccagagtcaccatgaccacagacacatccacgagcacagcctacatggagctgaggaacctgagatctgacgacacggccgtatattactgtgcgagagatagagtcgttgtagcagctgctaattactacttttattctatggacgtctggggccaagggaccacggtcaccgtctcctca

SEQ ID NO: 146 - protein, 126 aa, Artificial Sequence
  Synthetic
QVQLVQSGAEVKKPGASVKVSCKASGYTFTNYGISWVRQAPGQGLELMGWISGYNGNTNYAQELQARVTMTTDTSTSTAYMELRNLRSDDTAVYYCARDRVVVAAANYYFYSMDVWGQGTTVTVSS

SEQ ID NO: 147 - DNA, 24 bp, Artificial Sequence
  Synthetic
ggttacacctttaccaactatggt

SEQ ID NO: 148 - protein, 8 aa, Artificial Sequence
  Synthetic
GYTFTNYG

SEQ ID NO: 149 - DNA, 24 bp, Artificial Sequence
  Synthetic
attagtggttacaatggtaacaca

SEQ ID NO: 150 - protein, 8 aa, Artificial Sequence
  Synthetic
ISGYNGNT

SEQ ID NO: 151 - DNA, 57 bp, Artificial Sequence
  Synthetic
gcgagagatagagtcgttgtagcagctgctaattactacttttattctatggacgtc

SEQ ID NO: 152 - protein, 19 aa, Artificial Sequence
  Synthetic
ARDRVVVAAANYYFYSMDV

SEQ ID NO: 153 - DNA, 339 bp, Artificial Sequence
  Synthetic
gccatccagatgacccagtctccactctccctgtccgtcacccttggacagccggcctccatctcctgcaggtctagtcaaagcctcgtatacagtgatggagacacctacttgaattggtttcagcagaggccaggccaatctccaaggcgcctaatttataaggtttctaaccgggactctggggtcccagacagattcagcggcagtgggtcaggcactgctttcacactgaaaatcagcggggtggaggccgaggatgttggggtttactactgcatgcaagctacacactggcctcggacgttcggccaagggaccaaggtggaaatcaaacga

SEQ ID NO: 154 - protein, 113 aa, Artificial Sequence
  Synthetic
AIQMTQSPLSLSVTLGQPASISCRSSQSLVYSDGDTYLNWFQQRPGQSPRRLIYKVSNRDSGVPDRFSGSGSGTAFTLKISGVEAEDVGVYYCMQATHWPRTFGQGTKVEIKR

SEQ ID NO: 155 - DNA, 33 bp, Artificial Sequence
  Synthetic
caaagcctcgtatacagtgatggagacacctac

SEQ ID NO: 156 - protein, 11 aa, Artificial Sequence
  Synthetic
QSLVYSDGDTY

SEQ ID NO: 157 - DNA, 9 bp, Artificial Sequence
  Synthetic
aaggtttct

SEQ ID NO: 158 - protein, 3 aa, Artificial Sequence
  Synthetic
KVS

SEQ ID NO: 159 - DNA, 27 bp, Artificial Sequence
  Synthetic
atgcaagctacacactggcctcggacg

SEQ ID NO: 160 - protein, 9 aa, Artificial Sequence
  Synthetic
MQATHWPRT

SEQ ID NO: 161 - DNA, 378 bp, Artificial Sequence
  Synthetic
caggttcagctggtgcagtctggagctgaggtgaagaagcctggggcctcagtgaaggtctcctgcaaggcttctggttacacctttaccaactatggtatcagctgggtgcgacaggcccctggacaaggacttgagttaatgggatggattagtggttacaatggtaacacaaactatgcacaagaactccaggccagagtcaccatgaccacagacacatccacgagcacagcctacatggagctgaggaacctgagatctgacgacacggccgtatattactgtgcgagagatagagtcgttgtagcagctgctaattactacttttattctatggacgtctggggccaagggaccacggtcaccgtctcctca

SEQ ID NO: 162 - protein, 126 aa, Artificial Sequence; identical to SEQ ID NO: 146
  Synthetic

SEQ ID NO: 163 - DNA, 336 bp, Artificial Sequence
  Synthetic
gatgttgtgatgactcagtctccactctccctgtccgtcacccttggacagccggcctccatctcctgcaggtctagtcaaagcctcgtatacagtgatggagacacctacttgaattggtttcagcagaggccaggccaatctccaaggcgcctaatttataaggtttctaaccgggactctggggtcccagacagattcagcggcagtgggtcaggcactgctttcacactgaaaatcagcggggtggaggccgaggatgttggggtttactactgcatgcaagctacacactggcctcggacgttcggccaagggaccaaggtggaaatcaaa

SEQ ID NO: 164 - protein, 112 aa, Artificial Sequence
  Synthetic
DVVMTQSPLSLSVTLGQPASISCRSSQSLVYSDGDTYLNWFQQRPGQSPRRLIYKVSNRDSGVPDRFSGSGSGTAFTLKISGVEAEDVGVYYCMQATHWPRTFGQGTKVEIK

SEQ ID NO: 165 - DNA, 378 bp, Artificial Sequence
  Synthetic
caggttcagctggtgcagtctggagctgaggtgaagaagcctggggcctcagtgaaggtctcctgcaaggcttctggttacacctttaccaactatggtatcagctgggtgcgacaggcccctggacaagggcttgagtggatgggatggattagtggttacaatggtaacacaaactatgcacagaagctccagggcagagtcaccatgaccacagacacatccacgagcacagcctacatggagctgaggagcctgagatctgacgacacggccgtgtattactgtgcgagagatagagtcgttgtagcagctgctaattactacttttattctatggacgtctggggccaagggaccacggtcaccgtctcctca

SEQ ID NO: 166 - protein, 126 aa, Artificial Sequence
  Synthetic
QVQLVQSGAEVKKPGASVKVSCKASGYTFTNYGISWVRQAPGQGLEWMGWISGYNGNTNYAQKLQGRVTMTTDTSTSTAYMELRSLRSDDTAVYYCARDRVVVAAANYYFYSMDVWGQGTTVTVSS

SEQ ID NO: 167 - DNA, 336 bp, Artificial Sequence
  Synthetic
gatgttgtgatgactcagtctccactctccctgcccgtcacccttggacagccggcctccatctcctgcaggtctagtcaaagcctcgtatacagtgatggagacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaagctacacactggcctcggacgttcggccaagggaccaaggtggaaatcaaa

SEQ ID NO: 168 - protein, 112 aa, Artificial Sequence
  Synthetic
DVVMTQSPLSLPVTLGQPASISCRSSQSLVYSDGDTYLNWFQQRPGQSPRRLIYKVSNRDSGVPDRFSGSGSGTDFTLKISRVEAEDVGVYYCMQATHWPRTFGQGTKVEIK

SEQ ID NO: 169 - DNA, 375 bp, Artificial Sequence
  Synthetic
caggtccacttgaaggagtctggtcctacgctggtgaaacccacacagaccctcacgctgacctgcaccttctctggattctcactcatcactagtggagtgggtgtgggctggattcgtcagccccccggaaaggccctggagtggcttgcactcatttattggaatggtgataagcgctacagcccatctctgaagagcaggctcaccatcaccaaggacacctccaaaaaccaggtggtccttacaatgaccaacatggaccctgtggacacagccacatattactgtgcacacaggataactgaaactagttactacttctactacggtatggacgtctggggccaagggaccacggtcaccgtctcctca

SEQ ID NO: 170 - protein, 125 aa, Artificial Sequence
  Synthetic
QVHLKESGPTLVKPTQTLTLTCTFSGFSLITSGVGVGWIRQPPGKALEWLALIYWNGDKRYSPSLKSRLTITKDTSKNQVVLTMTNMDPVDTATYYCAHRITETSYYFYYGMDVWGQGTTVTVSS

SEQ ID NO: 171 - DNA, 30 bp, Artificial Sequence
  Synthetic
ggattctcactcatcactagtggagtgggt

SEQ ID NO: 172 - protein, 10 aa, Artificial Sequence
  Synthetic
GFSLITSGVG

SEQ ID NO: 173 - DNA, 21 bp, Artificial Sequence
  Synthetic
atttattggaatggtgataag

SEQ ID NO: 174 - protein, 7 aa, Artificial Sequence
  Synthetic
IYWNGDK

SEQ ID NO: 175 - DNA, 51 bp, Artificial Sequence
  Synthetic
gcacacaggataactgaaactagttactacttctactacggtatggacgtc

SEQ ID NO: 176 - protein, 17 aa, Artificial Sequence
  Synthetic
AHRITETSYYFYYGMDV

SEQ ID NO: 177 - DNA, 339 bp, Artificial Sequence
  Synthetic
gacatccagatgacccagtctccactctccctgcccgtcacccctggagagccggcctccatctcctgcaggtctagtcagagcctcctgcatagtcatggatacgactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgctcactttcggcggagggaccaaggtggaaatcaaacga

SEQ ID NO: 178 - protein, 113 aa, Artificial Sequence
  Synthetic
DIQMTQSPLSLPVTPGEPASISCRSSQSLLHSHGYDYLDWYLQKPGQSPQLLIYLGSNRASGVPDRFSGSGSGTDFTLKISRVEAEDVGVYYCMQALQTPLTFGGGTKVEIKR

SEQ ID NO: 179 - DNA, 33 bp, Artificial Sequence
  Synthetic
cagagcctcctgcatagtcatggatacgactat

SEQ ID NO: 180 - protein, 11 aa, Artificial Sequence
  Synthetic
QSLLHSHGYDY

SEQ ID NO: 181 - DNA, 9 bp, Artificial Sequence
  Synthetic
ttgggttct

SEQ ID NO: 182 - protein, 3 aa, Artificial Sequence
  Synthetic
LGS

SEQ ID NO: 183 - DNA, 27 bp, Artificial Sequence
  Synthetic
atgcaagctctacaaactccgctcact

SEQ ID NO: 184 - protein, 9 aa, Artificial Sequence
  Synthetic
MQALQTPLT

SEQ ID NO: 185 - DNA, 375 bp, Artificial Sequence
  Synthetic
cagatcaccttgaaggagtctggtcctacgctggtgaaacccacacagaccctcacgctgacctgcaccttctctggattctcactcatcactagtggagtgggtgtgggctggattcgtcagccccccggaaaggccctggagtggcttgcactcatttattggaatggtgataagcgctacagcccatctctgaagagcaggctcaccatcaccaaggacacctccaaaaaccaggtggtccttacaatgaccaacatggaccctgtggacacagccacatattactgtgcacacaggataactgaaactagttactacttctactacggtatggacgtctggggccaagggaccacggtcaccgtctcctca

SEQ ID NO: 186 - protein, 125 aa, Artificial Sequence
  Synthetic
QITLKESGPTLVKPTQTLTLTCTFSGFSLITSGVGVGWIRQPPGKALEWLALIYWNGDKRYSPSLKSRLTITKDTSKNQVVLTMTNMDPVDTATYYCAHRITETSYYFYYGMDVWGQGTTVTVSS

SEQ ID NO: 187 - DNA, 336 bp, Artificial Sequence
  Synthetic
gatattgtgatgactcagtctccactctccctgcccgtcacccctggagagccggcctccatctcctgcaggtctagtcagagcctcctgcatagtcatggatacgactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactccgctcactttcggcggagggaccaaggtggagatcaaa

SEQ ID NO: 188 - protein, 112 aa, Artificial Sequence
  Synthetic
DIVMTQSPLSLPVTPGEPASISCRSSQSLLHSHGYDYLDWYLQKPGQSPQLLIYLGSNRASGVPDRFSGSGSGTDFTLKISRVEAEDVGVYYCMQALQTPLTFGGGTKVEIK

SEQ ID NO: 189 - DNA, 375 bp, Artificial Sequence
  Synthetic
cagatcaccttgaaggagtctggtcctacgctggtgaaacccacacagaccctcacgctgacctgcaccttctctggattctcactcatcactagtggagtgggtgtgggctggatccgtcagcccccaggaaaggccctggagtggcttgcactcatttattggaatggtgataagcgctacagcccatctctgaagagcaggctcaccatcaccaaggacacctccaaaaaccaggtggtccttacaatgaccaacatggaccctgtggacacagccacatattactgtgcacacaggataactgaaactagttactacttctactacggtatggacgtctggggccaagggaccacggtcaccgtctcctca

SEQ ID NO: 190 - protein, 125 aa, Artificial Sequence; identical to SEQ ID NO: 186
  Synthetic

SEQ ID NO: 191 - DNA, 336 bp, Artificial Sequence; identical to SEQ ID NO: 187
  Synthetic

SEQ ID NO: 192 - protein, 112 aa, Artificial Sequence; identical to SEQ ID NO: 188
  Synthetic

SEQ ID NO: 193 - DNA, 375 bp, Artificial Sequence
  Synthetic
cagatcaccttgaaggagtctggtcctactctggtgaaaccctcacagaccctcacgctgacctgcaccttctctgggttctcactcagcactagtggagtgggtgtgggctggatccgtcagcccccaggaaaggccctggagtggcttgcactcatttattggaattctgataagcgctacagcccatctctgaagagcaggctcaccatcaccaaggacacctccaaaaaccaggtagtccttacaatgaccaacatggaccctgtggacacagccacatattactgtgcacacagacatgacagctcgtcctactacttctactacggtatggacgtctggggccaagggatcacggtcaccgtctcctca

SEQ ID NO: 194 - protein, 125 aa, Artificial Sequence
  Synthetic
QITLKESGPTLVKPSQTLTLTCTFSGFSLSTSGVGVGWIRQPPGKALEWLALIYWNSDKRYSPSLKSRLTITKDTSKNQVVLTMTNMDPVDTATYYCAHRHDSSSYYFYYGMDVWGQGITVTVSS

SEQ ID NO: 195 - DNA, 30 bp, Artificial Sequence
  Synthetic
gggttctcactcagcactagtggagtgggt

SEQ ID NO: 196 - protein, 10 aa, Artificial Sequence
  Synthetic
GFSLSTSGVG

SEQ ID NO: 197 - DNA, 21 bp, Artificial Sequence
  Synthetic
atttattggaattctgataag

SEQ ID NO: 198 - protein, 7 aa, Artificial Sequence
  Synthetic
IYWNSDK

SEQ ID NO: 199 - DNA, 51 bp, Artificial Sequence
  Synthetic
gcacacagacatgacagctcgtcctactacttctactacggtatggacgtc

SEQ ID NO: 200 - protein, 17 aa, Artificial Sequence
  Synthetic
AHRHDSSSYYFYYGMDV

SEQ ID NO: 201 - DNA, 339 bp, Artificial Sequence
  Synthetic
gacatccagatgacccagtctccgctctccctgcccgtcacccctggagagccggcctccatctcctgcaggtctagtcagagcctcctccatagtcatggatacaactatttggattggtacctgcagaagccagggcagtctccacaactcctgatctatttgggttctaatcgggcctccggggtccctgacaggttcagtggcggtggatcaggcacagattttacactgaaaatcagcagagtggaggctgaggatgttgggatttattactgcatgcaagctctacagactcctctcactttcggcggagggaccaaggtggagatcaaacga

SEQ ID NO: 202 - protein, 113 aa, Artificial Sequence
  Synthetic
DIQMTQSPLSLPVTPGEPASISCRSSQSLLHSHGYNYLDWYLQKPGQSPQLLIYLGSNRASGVPDRFSGGGSGTDFTLKISRVEAEDVGIYYCMQALQTPLTFGGGTKVEIKR

SEQ ID NO: 203 - DNA, 33 bp, Artificial Sequence
  Synthetic
cagagcctcctccatagtcatggatacaactat

SEQ ID NO: 204 - protein, 11 aa, Artificial Sequence
  Synthetic
QSLLHSHGYNY

SEQ ID NO: 205 - DNA, 9 bp, Artificial Sequence
  Synthetic
ttgggttct

SEQ ID NO: 206 - protein, 3 aa, Artificial Sequence
  Synthetic
LGS

SEQ ID NO: 207 - DNA, 27 bp, Artificial Sequence
  Synthetic
atgcaagctctacagactcctctcact

SEQ ID NO: 208 - protein, 9 aa, Artificial Sequence
  Synthetic
MQALQTPLT

SEQ ID NO: 209 - DNA, 375 bp, Artificial Sequence
  Synthetic
cagatcaccttgaaggagtctggtcctactctggtgaaaccctcacagaccctcacgctgacctgcaccttctctgggttctcactcagcactagtggagtgggtgtgggctggatccgtcagcccccaggaaaggccctggagtggcttgcactcatttattggaattctgataagcgctacagcccatctctgaagagcaggctcaccatcaccaaggacacctccaaaaaccaggtagtccttacaatgaccaacatggaccctgtggacacagccacatattactgtgcacacagacatgacagctcgtcctactacttctactacggtatggacgtctggggccaagggaccacggtcaccgtctcctca

SEQ ID NO: 210 - protein, 125 aa, Artificial Sequence
  Synthetic
QITLKESGPTLVKPSQTLTLTCTFSGFSLSTSGVGVGWIRQPPGKALEWLALIYWNSDKRYSPSLKSRLTITKDTSKNQVVLTMTNMDPVDTATYYCAHRHDSSSYYFYYGMDVWGQGTTVTVSS

SEQ ID NO: 211 - DNA, 336 bp, Artificial Sequence
  Synthetic
gatattgtgatgactcagtctccgctctccctgcccgtcacccctggagagccggcctccatctcctgcaggtctagtcagagcctcctccatagtcatggatacaactatttggattggtacctgcagaagccagggcagtctccacaactcctgatctatttgggttctaatcgggcctccggggtccctgacaggttcagtggcggtggatcaggcacagattttacactgaaaatcagcagagtggaggctgaggatgttgggatttattactgcatgcaagctctacagactcctctcactttcggcggagggaccaaggtggagatcaaa

SEQ ID NO: 212 - protein, 112 aa, Artificial Sequence
  Synthetic
DIVMTQSPLSLPVTPGEPASISCRSSQSLLHSHGYNYLDWYLQKPGQSPQLLIYLGSNRASGVPDRFSGGGSGTDFTLKISRVEAEDVGIYYCMQALQTPLTFGGGTKVEIK

SEQ ID NO: 213 - DNA, 375 bp, Artificial Sequence
  Synthetic
cagatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcactcatttattggaattctgataagcgctacagcccatctctgaagagcaggctcaccatcaccaaggacacctccaaaaaccaggtggtccttacaatgaccaacatggaccctgtggacacagccacatattactgtgcacacagacatgacagctcgtcctactacttctactacggtatggacgtctggggccaagggaccacggtcaccgtctcctca

SEQ ID NO: 214 - protein, 125 aa, Artificial Sequence
  Synthetic
QITLKESGPTLVKPTQTLTLTCTFSGFSLSTSGVGVGWIRQPPGKALEWLALIYWNSDKRYSPSLKSRLTITKDTSKNQVVLTMTNMDPVDTATYYCAHRHDSSSYYFYYGMDVWGQGTTVTVSS

SEQ ID NO: 215 - DNA, 336 bp, Artificial Sequence
  Synthetic
gatattgtgatgactcagtctccactctccctgcccgtcacccctggagagccggcctccatctcctgcaggtctagtcagagcctcctccatagtcatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacagactcctctcactttcggcggagggaccaaggtggagatcaaa

SEQ ID NO: 216 - protein, 112 aa, Artificial Sequence
  Synthetic
DIVMTQSPLSLPVTPGEPASISCRSSQSLLHSHGYNYLDWYLQKPGQSPQLLIYLGSNRASGVPDRFSGSGSGTDFTLKISRVEAEDVGVYYCMQALQTPLTFGGGTKVEIK

SEQ ID NO: 217 - DNA, 381 bp, Artificial Sequence
  Synthetic
gagatgcaactggtggagtctgggggaggcttggtccagcctggggggtccctgagactctcctgtgcagcctctggattcacctttagtagtcactggatgaagtgggtccgccaggctccagggaaggggctggagtgggtggccaacataaaccaagatggaagtgagaaatactatgtggactctgtgaagggccgattcaccatctccagagacaacgccaagaactcactgtttctgcaaatgaacagcctgagagccgaggacacggctgtgtattactgtgcgagagatattgtactaatggtctatgatatggactactactactacggtatggacgtctggggccaagggaccacggtcaccgtctcctca

SEQ ID NO: 218 - protein, 127 aa, Artificial Sequence
  Synthetic
EMQLVESGGGLVQPGGSLRLSCAASGFTFSSHWMKWVRQAPGKGLEWVANINQDGSEKYYVDSVKGRFTISRDNAKNSLFLQMNSLRAEDTAVYYCARDIVLMVYDMDYYYYGMDVWGQGTTVTVSS

SEQ ID NO: 219 - DNA, 24 bp, Artificial Sequence
  Synthetic
ggattcacctttagtagtcactgg

SEQ ID NO: 220 - protein, 8 aa, Artificial Sequence
  Synthetic
GFTFSSHW

SEQ ID NO: 221 - DNA, 24 bp, Artificial Sequence
  Synthetic
ataaaccaagatggaagtgagaaa

SEQ ID NO: 222 - protein, 8 aa, Artificial Sequence
  Synthetic
INQDGSEK

SEQ ID NO: 223 - DNA, 60 bp, Artificial Sequence
  Synthetic
gcgagagatattgtactaatggtctatgatatggactactactactacggtatggacgtc

SEQ ID NO: 224 - protein, 20 aa, Artificial Sequence
  Synthetic
ARDIVLMVYDMDYYYYGMDV

SEQ ID NO: 225 - DNA, 336 bp, Artificial Sequence
  Synthetic
gatattgtgatgactcagtctccactctccctgcccgtcacccctggagagccggcctccatctcctgcaggtctagtcagagcctcctgcatagtaatggaaacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaaactctacaaactccgctcactttcggcggagggaccaaggtggagatcaaa

SEQ ID NO: 226 - protein, 112 aa, Artificial Sequence
  Synthetic
DIVMTQSPLSLPVTPGEPASISCRSSQSLLHSNGNNYLDWYLQKPGQSPQLLIYLGSNRASGVPDRFSGSGSGTDFTLKISRVEAEDVGVYYCMQTLQTPLTFGGGTKVEIK

SEQ ID NO: 227 - DNA, 33 bp, Artificial Sequence
  Synthetic
cagagcctcctgcatagtaatggaaacaactat

SEQ ID NO: 228 - protein, 11 aa, Artificial Sequence
  Synthetic
QSLLHSNGNNY

SEQ ID NO: 229 - DNA, 9 bp, Artificial Sequence
  Synthetic
ttgggttct

SEQ ID NO: 230 - protein, 3 aa, Artificial Sequence
  Synthetic
LGS

SEQ ID NO: 231 - DNA, 27 bp, Artificial Sequence
  Synthetic
atgcaaactctacaaactccgctcact

SEQ ID NO: 232 - protein, 9 aa, Artificial Sequence
  Synthetic
MQTLQTPLT

SEQ ID NO: 233 - DNA, 381 bp, Artificial Sequence
  Synthetic
gaggtgcagctggtggagtctgggggaggcttggtccagcctggggggtccctgagactctcctgtgcagcctctggattcacctttagtagtcactggatgaagtgggtccgccaggctccagggaaggggctggagtgggtggccaacataaaccaagatggaagtgagaaatactatgtggactctgtgaagggccgattcaccatctccagagacaacgccaagaactcactgtttctgcaaatgaacagcctgagagccgaggacacggctgtgtattactgtgcgagagatattgtactaatggtctatgatatggactactactactacggtatggacgtctggggccaagggaccacggtcaccgtctcctca

SEQ ID NO: 234 - protein, 127 aa, Artificial Sequence
  Synthetic
EVQLVESGGGLVQPGGSLRLSCAASGFTFSSHWMKWVRQAPGKGLEWVANINQDGSEKYYVDSVKGRFTISRDNAKNSLFLQMNSLRAEDTAVYYCARDIVLMVYDMDYYYYGMDVWGQGTTVTVSS

SEQ ID NO: 235 - DNA, 336 bp, Artificial Sequence; identical to SEQ ID NO: 225
  Synthetic

SEQ ID NO: 236 - protein, 112 aa, Artificial Sequence; identical to SEQ ID NO: 226
  Synthetic

SEQ ID NO: 237 - DNA, 381 bp, Artificial Sequence
  Synthetic
gaggtgcagctggtggagtctgggggaggcttggtccagcctggggggtccctgagactctcctgtgcagcctctggattcacctttagtagtcactggatgagctgggtccgccaggctccagggaaggggctggagtgggtggccaacataaaccaagatggaagtgagaaatactatgtggactctgtgaagggccgattcaccatctccagagacaacgccaagaactcactgtatctgcaaatgaacagcctgagagccgaggacacggctgtgtattactgtgcgagagatattgtactaatggtctatgatatggactactactactacggtatggacgtctgggggcaagggaccacggtcaccgtctcctca

SEQ ID NO: 238 - protein, 127 aa, Artificial Sequence
  Synthetic
EVQLVESGGGLVQPGGSLRLSCAASGFTFSSHWMSWVRQAPGKGLEWVANINQDGSEKYYVDSVKGRFTISRDNAKNSLYLQMNSLRAEDTAVYYCARDIVLMVYDMDYYYYGMDVWGQGTTVTVSS

SEQ ID NO: 239 - DNA, 336 bp, Artificial Sequence; identical to SEQ ID NO: 225
  Synthetic

SEQ ID NO: 240 - protein, 112 aa, Artificial Sequence; identical to SEQ ID NO: 226
  Synthetic

SEQ ID NO: 241 - DNA, 381 bp, Artificial Sequence
  Synthetic
caggtgcagctggtggagtctgggggaggcgtggtccagcctgggaggtccctgagactctcctgtgcagtctctggattcaccttcagtagctatggcatgcactgggtccgccaggctccaggcaaggggctggagtgggtggcagctatatcatatgatggaagtaataaatactatgtagactccgtgaagggccgattcaccatctccagagacaattccaagaaaacgctgtatctgcaaatgaacagcctgagagctgaggacacggctgtgtataattgtgcgaaaaatattgtactagtgatgtatgatatagactatcactactatgggatggacgtctggggccaagggaccacggtcaccgtctcctca

SEQ ID NO: 242 - protein, 127 aa, Artificial Sequence
  Synthetic
QVQLVESGGGVVQPGRSLRLSCAVSGFTFSSYGMHWVRQAPGKGLEWVAAISYDGSNKYYVDSVKGRFTISRDNSKKTLYLQMNSLRAEDTAVYNCAKNIVLVMYDIDYHYYGMDVWGQGTTVTVSS

SEQ ID NO: 243 - DNA, 24 bp, Artificial Sequence
  Synthetic
ggattcaccttcagtagctatggc

SEQ ID NO: 244 - protein, 8 aa, Artificial Sequence
  Synthetic
GFTFSSYG

SEQ ID NO: 245 - DNA, 24 bp, Artificial Sequence
  Synthetic
atatcatatgatggaagtaataaa

SEQ ID NO: 246 - protein, 8 aa, Artificial Sequence
  Synthetic
ISYDGSNK

SEQ ID NO: 247 - DNA, 60 bp, Artificial Sequence
  Synthetic
gcgaaaaatattgtactagtgatgtatgatatagactatcactactatgggatggacgtc

SEQ ID NO: 248 - protein, 20 aa, Artificial Sequence
  Synthetic
AKNIVLVMYDIDYHYYGMDV

SEQ ID NO: 249 - DNA, 336 bp, Artificial Sequence
  Synthetic
gatattgtgatgactcagtctccactctccctgcccgtcacccctggagagccggcctccatctcctgcaggtctagtcagagcctcctgcatagtaatggatacaactatttggattggtacctgcagaagccagggcagtctccacaactcctgatctatttgggttttaatcgggcctccggggtccctgacaggttcagtggcagtggatcaggcacagattttacactgaaaatcagcagagtggaggctgaggatgttggggtttattactgcatgcaagctctacaaactcctctcactttcggcggagggaccaaggtggagatcaga

SEQ ID NO: 250 - protein, 112 aa, Artificial Sequence
  Synthetic
DIVMTQSPLSLPVTPGEPASISCRSSQSLLHSNGYNYLDWYLQKPGQSPQLLIYLGFNRASGVPDRFSGSGSGTDFTLKISRVEAEDVGVYYCMQALQTPLTFGGGTKVEIR

SEQ ID NO: 251 - DNA, 33 bp, Artificial Sequence
  Synthetic
cagagcctcctgcatagtaatggatacaactat

SEQ ID NO: 252 - protein, 11 aa, Artificial Sequence
  Synthetic
QSLLHSNGYNY

SEQ ID NO: 253 - DNA, 9 bp, Artificial Sequence
  Synthetic
ttgggtttt

SEQ ID NO: 254 - protein, 3 aa, Artificial Sequence
  Synthetic
LGF

SEQ ID NO: 255 - DNA, 27 bp, Artificial Sequence
  Synthetic
atgcaagctctacaaactcctctcact

SEQ ID NO: 256 - protein, 9 aa, Artificial Sequence
  Synthetic
MQALQTPLT

SEQ ID NO: 257 - DNA, 381 bp, Artificial Sequence; identical to SEQ ID NO: 241
  Synthetic

SEQ ID NO: 258 - protein, 127 aa, Artificial Sequence; identical to SEQ ID NO: 242
  Synthetic

SEQ ID NO: 259 - DNA, 336 bp, Artificial Sequence
  Synthetic
gatattgtgatgactcagtctccactctccctgcccgtcacccctggagagccggcctccatctcctgcaggtctagtcagagcctcctgcatagtaatggatacaactatttggattggtacctgcagaagccagggcagtctccacaactcctgatctatttgggttttaatcgggcctccggggtccctgacaggttcagtggcagtggatcaggcacagattttacactgaaaatcagcagagtggaggctgaggatgttggggtttattactgcatgcaagctctacaaactcctctcactttcggcggagggaccaaggtggagatcaaa

SEQ ID NO: 260 - protein, 112 aa, Artificial Sequence
  Synthetic
DIVMTQSPLSLPVTPGEPASISCRSSQSLLHSNGYNYLDWYLQKPGQSPQLLIYLGFNRASGVPDRFSGSGSGTDFTLKISRVEAEDVGVYYCMQALQTPLTFGGGTKVEIK

SEQ ID NO: 261 - DNA, 381 bp, Artificial Sequence
  Synthetic
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaaaatattgtactagtgatgtatgatatagactatcactactatgggatggacgtctgggggcaagggaccacggtcaccgtctcctca

SEQ ID NO: 262 - protein, 127 aa, Artificial Sequence
  Synthetic
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKNIVLVMYDIDYHYYGMDVWGQGTTVTVSS

SEQ ID NO: 263 - DNA, 336 bp, Artificial Sequence
  Synthetic
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttttaatcgggcctccggggtccctgacaggttcagtggcagtggatcaggcacagattttacactgaaaatcagcagagtggaggctgaggatgttggggtttattactgcatgcaagctctacaaactcctctcactttcggcggagggaccaaggtggagatcaaa

SEQ ID NO: 264 - protein, 112 aa, Artificial Sequence; identical to SEQ ID NO: 260
  Synthetic

SEQ ID NO: 265 - DNA, 381 bp, Artificial Sequence; identical to SEQ ID NO: 241
  Synthetic

SEQ ID NO: 266 - protein, 127 aa, Artificial Sequence; identical to SEQ ID NO: 242
  Synthetic

SEQ ID NO: 267 - DNA, 24 bp, Artificial Sequence
  Synthetic
ggattcaccttcagtagctatggc

SEQ ID NO: 268 - protein, 8 aa, Artificial Sequence
  Synthetic
GFTFSSYG

SEQ ID NO: 269 - DNA, 24 bp, Artificial Sequence
  Synthetic
atatcatatgatggaagtaataaa

SEQ ID NO: 270 - protein, 8 aa, Artificial Sequence
  Synthetic
ISYDGSNK

SEQ ID NO: 271 - DNA, 60 bp, Artificial Sequence; identical to SEQ ID NO: 247
  Synthetic

SEQ ID NO: 272 - protein, 20 aa, Artificial Sequence
  Synthetic
AKNIVLVMYDIDYHYYGMDV

SEQ ID NO: 273 - DNA, 336 bp, Artificial Sequence; identical to SEQ ID NO: 249
  Synthetic

SEQ ID NO: 274 - protein, 112 aa, Artificial Sequence; identical to SEQ ID NO: 250
  Synthetic

SEQ ID NO: 275 - DNA, 33 bp, Artificial Sequence; identical to SEQ ID NO: 251
  Synthetic

SEQ ID NO: 276 - protein, 11 aa, Artificial Sequence
  Synthetic
QSLLHSNGYNY

SEQ ID NO: 277 - DNA, 9 bp, Artificial Sequence
  Synthetic
ttgggtttt

SEQ ID NO: 278 - protein, 3 aa, Artificial Sequence
  Synthetic
LGF

SEQ ID NO: 279 - DNA, 27 bp, Artificial Sequence
  Synthetic
atgcaagctctacaaactcctctcact

SEQ ID NO: 280 - protein, 9 aa, Artificial Sequence
  Synthetic
MQALQTPLT

SEQ ID NO: 281 - DNA, 381 bp, Artificial Sequence; identical to SEQ ID NO: 241
  Synthetic

SEQ ID NO: 282 - protein, 127 aa, Artificial Sequence; identical to SEQ ID NO: 242
  Synthetic

SEQ ID NO: 283 - DNA, 336 bp, Artificial Sequence; identical to SEQ ID NO: 259
  Synthetic

SEQ ID NO: 284 - protein, 112 aa, Artificial Sequence; identical to SEQ ID NO: 260
  Synthetic

SEQ ID NO: 285 - DNA, 381 bp, Artificial Sequence; identical to SEQ ID NO: 261
  Synthetic

SEQ ID NO: 286 - protein, 127 aa, Artificial Sequence; identical to SEQ ID NO: 262
  Synthetic

SEQ ID NO: 287 - DNA, 336 bp, Artificial Sequence; identical to SEQ ID NO: 263
  Synthetic

SEQ ID NO: 288 - protein, 112 aa, Artificial Sequence; identical to SEQ ID NO: 260
  Synthetic

SEQ ID NO: 289 - DNA, 372 bp, Artificial Sequence
  Synthetic
cagatcaccttgaaggagtctggtcctacgctggtaaaacccacacagaccctcacgctgacctgcaccttctctgggttctcactcagcgctagtggagtgggtgtgggctggttccgtcagcccccaggaaaggccctggagtggcttgcactcatttattggaatgatgataagcgttacagcccatctctaaagaacagcctcaccatcaccaaggacacctccaaaaaccaggtggtccttacaatgaccaacatggaccctgtggacacagccacatattactgtgcacacagaatacatctatggtcctacttctactacggtatggacgtctggggccaagggaccacggtcaccgtctcctca

SEQ ID NO: 290 - protein, 124 aa, Artificial Sequence
  Synthetic
QITLKESGPTLVKPTQTLTLTCTFSGFSLSASGVGVGWFRQPPGKALEWLALIYWNDDKRYSPSLKNSLTITKDTSKNQVVLTMTNMDPVDTATYYCAHRIHLWSYFYYGMDVWGQGTTVTVSS

SEQ ID NO: 291 - DNA, 30 bp, Artificial Sequence
  Synthetic
gggttctcactcagcgctagtggagtgggt

SEQ ID NO: 292 - protein, 10 aa, Artificial Sequence
  Synthetic
GFSLSASGVG

SEQ ID NO: 293 - DNA, 21 bp, Artificial Sequence
  Synthetic
atttattggaatgatgataag

SEQ ID NO: 294 - protein, 7 aa, Artificial Sequence
  Synthetic
IYWNDDK

SEQ ID NO: 295 - DNA, 48 bp, Artificial Sequence
  Synthetic
gcacacagaatacatctatggtcctacttctactacggtatggacgtc

SEQ ID NO: 296 - protein, 16 aa, Artificial Sequence
  Synthetic
AHRIHLWSYFYYGMDV

SEQ ID NO: 297 - DNA, 336 bp, Artificial Sequence
  Synthetic
gatattgtgatgactcagtctccactctccctgcccgtcacccctggagagccggcctccatctcctgcaggtctagtcagactctcctgcatagtaatggatacaactatttcgattggtacctgcagaagccagggcagtctccacagctcctgatctatttgggttctaatcgggcctccggggtccctgacagattcagtggcagtggatcaggcacagattttacactgaaaatcagcagagtggaggctgaggatgttggaatttattactgcatgcaagctctacaaactcctctcactttcggcggagggaccaaggtggagatcaga

SEQ ID NO: 298 - protein, 112 aa, Artificial Sequence
  Synthetic
DIVMTQSPLSLPVTPGEPASISCRSSQTLLHSNGYNYFDWYLQKPGQSPQLLIYLGSNRASGVPDRFSGSGSGTDFTLKISRVEAEDVGIYYCMQALQTPLTFGGGTKVEIR

SEQ ID NO: 299 - DNA, 33 bp, Artificial Sequence
  Synthetic
cagactctcctgcatagtaatggatacaactat

SEQ ID NO: 300 - protein, 11 aa, Artificial Sequence
  Synthetic
QTLLHSNGYNY

SEQ ID NO: 301 - DNA, 9 bp, Artificial Sequence
  Synthetic
ttgggttct

SEQ ID NO: 302 - protein, 3 aa, Artificial Sequence
  Synthetic
LGS

SEQ ID NO: 303 - DNA, 27 bp, Artificial Sequence
  Synthetic
atgcaagctctacaaactcctctcact

SEQ ID NO: 304 - protein, 9 aa, Artificial Sequence
  Synthetic
MQALQTPLT

SEQ ID NO: 305 - DNA, 372 bp, Artificial Sequence; identical to SEQ ID NO: 289
  Synthetic

SEQ ID NO: 306 - protein, 124 aa, Artificial Sequence; identical to SEQ ID NO: 290
  Synthetic

SEQ ID NO: 307 - DNA, 336 bp, Artificial Sequence
  Synthetic
gatattgtgatgactcagtctccactctccctgcccgtcacccctggagagccggcctccatctcctgcaggtctagtcagactctcctgcatagtaatggatacaactatttcgattggtacctgcagaagccagggcagtctccacagctcctgatctatttgggttctaatcgggcctccggggtccctgacagattcagtggcagtggatcaggcacagattttacactgaaaatcagcagagtggaggctgaggatgttggaatttattactgcatgcaagctctacaaactcctctcactttcggcggagggaccaaggtggagatcaaa

SEQ ID NO: 308 - protein, 112 aa, Artificial Sequence
  Synthetic
DIVMTQSPLSLPVTPGEPASISCRSSQTLLHSNGYNYFDWYLQKPGQSPQLLIYLGSNRASGVPDRFSGSGSGTDFTLKISRVEAEDVGIYYCMQALQTPLTFGGGTKVEIK

SEQ ID NO: 309 - DNA, 372 bp, Artificial Sequence
  Synthetic
cagatcaccttgaaggagtctggtcctacgctggtgaaacccacacagaccctcacgctgacctgcaccttctctgggttctcactcagcgctagtggagtgggtgtgggctggatccgtcagcccccaggaaaggccctggagtggcttgcactcatttattggaatgatgataagcgctacagcccatctctgaagagcaggctcaccatcaccaaggacacctccaaaaaccaggtggtccttacaatgaccaacatggaccctgtggacacagccacatattactgtgcacacagaatacatctatggtcctacttctactacggtatggacgtctgggggcaagggaccacggtcaccgtctcctca

SEQ ID NO: 310 - protein, 124 aa, Artificial Sequence
  Synthetic
QITLKESGPTLVKPTQTLTLTCTFSGFSLSASGVGVGWIRQPPGKALEWLALIYWNDDKRYSPSLKSRLTITKDTSKNQVVLTMTNMDPVDTATYYCAHRIHLWSYFYYGMDVWGQGTTVTVSS

SEQ ID NO: 311 - DNA, 336 bp, Artificial Sequence
  Synthetic
gatattgtgatgactcagtctccactctccctgcccgtcacccctggagagccggcctccatctcctgcaggtctagtcagactctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctctcactttcggcggagggaccaaggtggagatcaaa

SEQ ID NO: 312 - protein, 112 aa, Artificial Sequence
  Synthetic
DIVMTQSPLSLPVTPGEPASISCRSSQTLLHSNGYNYLDWYLQKPGQSPQLLIYLGSNRASGVPDRFSGSGSGTDFTLKISRVEAEDVGVYYCMQALQTPLTFGGGTKVEIK

SEQ ID NO: 313 - DNA, 381 bp, Artificial Sequence
  Synthetic
caggttcagctggtgcagtctggacctgaggtgaagaaccctggggcctcagtgaaggtctcctgcaaggcttctggttacacctttaccacctatggtatcagttgggtacgacaggcccctggacaagggcttgagtggatgggatggatcagcggttacaatggtaaaacaaacgatgcacagaagttccaggacagagtcgccatgaccacagacacatccacgagcacagcctacatggagctgaggagcctgagatctgacgacacggccatttattactgttcgagagatcgtttagtagtaccacctgcccttaattattcctactacgttatggacgtctggggccaagggaccacggtcaccgtctcctca

SEQ ID NO: 314 - protein, 127 aa, Artificial Sequence
  Synthetic
QVQLVQSGPEVKNPGASVKVSCKASGYTFTTYGISWVRQAPGQGLEWMGWISGYNGKTNDAQKFQDRVAMTTDTSTSTAYMELRSLRSDDTAIYYCSRDRLVVPPALNYSYYVMDVWGQGTTVTVSS

SEQ ID NO: 315 - DNA, 24 bp, Artificial Sequence
  Synthetic
ggttacacctttaccacctatggt

SEQ ID NO: 316 - protein, 8 aa, Artificial Sequence
  Synthetic
GYTFTTYG

SEQ ID NO: 317 - DNA, 24 bp, Artificial Sequence
  Synthetic
atcagcggttacaatggtaaaaca

SEQ ID NO: 318 - protein, 8 aa, Artificial Sequence
  Synthetic
ISGYNGKT

SEQ ID NO: 319 - DNA, 60 bp, Artificial Sequence
  Synthetic
tcgagagatcgtttagtagtaccacctgcccttaattattcctactacgttatggacgtc

SEQ ID NO: 320 - protein, 20 aa, Artificial Sequence
  Synthetic
SRDRLVVPPALNYSYYVMDV

SEQ ID NO: 321 - DNA, 336 bp, Artificial Sequence
  Synthetic
gatgttgtgatgactcagtctccactctccctgcccgtcacccttggacagccggcctccatctcctgcaggtctagtcaaagcctcgtatacagtgatggaaacacctacttgaattggtctcagcagaggccaggtcaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccgtacacttttggccaggggaccaagctggagatcaaa

SEQ ID NO: 322 - protein, 112 aa, Artificial Sequence
  Synthetic
DVVMTQSPLSLPVTLGQPASISCRSSQSLVYSDGNTYLNWSQQRPGQSPRRLIYKVSNRDSGVPDRFSGSGSGTDFTLKISRVEAEDVGVYYCMQGTHWPYTFGQGTKLEIK

SEQ ID NO: 323 - DNA, 33 bp, Artificial Sequence
  Synthetic
caaagcctcgtatacagtgatggaaacacctac

SEQ ID NO: 324 - protein, 11 aa, Artificial Sequence
  Synthetic
QSLVYSDGNTY

SEQ ID NO: 325 - DNA, 9 bp, Artificial Sequence
  Synthetic
aaggtttct

SEQ ID NO: 326 - protein, 3 aa, Artificial Sequence
  Synthetic
KVS

SEQ ID NO: 327 - DNA, 27 bp, Artificial Sequence
  Synthetic
atgcaaggtacacactggccgtacact

SEQ ID NO: 328 - protein, 9 aa, Artificial Sequence
  Synthetic
MQGTHWPYT

SEQ ID NO: 329 - DNA, 381 bp, Artificial Sequence; identical to SEQ ID NO: 313
  Synthetic

SEQ ID NO: 330 - protein, 127 aa, Artificial Sequence; identical to SEQ ID NO: 314
  Synthetic

SEQ ID NO: 331 - DNA, 336 bp, Artificial Sequence; identical to SEQ ID NO: 321
  Synthetic

SEQ ID NO: 332 - protein, 112 aa, Artificial Sequence; identical to SEQ ID NO: 322
  Synthetic

SEQ ID NO: 333 - DNA, 381 bp, Artificial Sequence
  Synthetic
caggttcagctggtgcagtctggagctgaggtgaagaagcctggggcctcagtgaaggtctcctgcaaggcttctggttacacctttaccacctatggtatcagctgggtgcgacaggcccctggacaagggcttgagtggatgggatggatcagcggttacaatggtaaaacaaactatgcacagaagctccagggcagagtcaccatgaccacagacacatccacgagcacagcctacatggagctgaggagcctgagatctgacgacacggccgtgtattactgttcgagagatcgtttagtagtaccacctgcccttaattattcctactacgttatggacgtctgggggcaagggaccacggtcaccgtctcctca

SEQ ID NO: 334 - protein, 127 aa, Artificial Sequence
  Synthetic
QVQLVQSGAEVKKPGASVKVSCKASGYTFTTYGISWVRQAPGQGLEWMGWISGYNGKTNYAQKLQGRVTMTTDTSTSTAYMELRSLRSDDTAVYYCSRDRLVVPPALNYSYYVMDVWGQGTTVTVSS

SEQ ID NO: 335 - DNA, 336 bp, Artificial Sequence
  Synthetic
gatgttgtgatgactcagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccgtacacttttggccaggggaccaagctggagatcaaa

SEQ ID NO: 336 - protein, 112 aa, Artificial Sequence
  Synthetic
DVVMTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPYTFGQGTKLEIK

SEQ ID NO: 337 - DNA, 354 bp, Artificial Sequence
  Synthetic
gaggtgcagctggtggagtctgggggaggcctggtcaagcctggggggtccctgagactctcctgtgcagcctctggattcaccttcagtagctatagcatggactgggtccgccaggctccagggaaggggctggagtgggtctcatccattagtagtagtagtagttacatatactacgcagactctgtgaagggccgattcaccatctccagagacaccgccaagaactcactgtatctgcaaatgaacagcctgagagacgaggacacggctgtttattactgtgcgagagagggcagtagcagactttttgactactggggccagggaaccctggtcaccgtctcctca

SEQ ID NO: 338 - protein, 118 aa, Artificial Sequence
  Synthetic
EVQLVESGGGLVKPGGSLRLSCAASGFTFSSYSMDWVRQAPGKGLEWVSSISSSSSYIYYADSVKGRFTISRDTAKNSLYLQMNSLRDEDTAVYYCAREGSSRLFDYWGQGTLVTVSS

SEQ ID NO: 339 - DNA, 24 bp, Artificial Sequence
  Synthetic
ggattcaccttcagtagctatagc

SEQ ID NO: 340 - protein, 8 aa, Artificial Sequence
  Synthetic
GFTFSSYS

SEQ ID NO: 341 - DNA, 24 bp, Artificial Sequence
  Synthetic
attagtagtagtagtagttacata

SEQ ID NO: 342 - protein, 8 aa, Artificial Sequence
  Synthetic
ISSSSSYI

SEQ ID NO: 343 - DNA, 33 bp, Artificial Sequence
  Synthetic
gcgagagagggcagtagcagactttttgactac

SEQ ID NO: 344 - protein, 11 aa, Artificial Sequence
  Synthetic
AREGSSRLFDY

SEQ ID NO: 345 - DNA, 321 bp, Artificial Sequence
  Synthetic
gacatccagatgacccagtctccttccaccctgtctgcatctgtaggagacagagtcaccatcacttgccgggccagtcagagtattagtagctggttggcctggtatcagcagagaccagggaaagcccctaagctcctgatctataaggcgtctagtttagaaggtggagtcccatcaaggttcagcggcagtggatctgggacagaattcactctcaccatcagcagcctgcagcctgaggattttgcaacttattactgccaacagtataatagttattggtacacttttggccaggggaccaagctggagatcaaa

SEQ ID NO: 346 - protein, 107 aa, Artificial Sequence
  Synthetic
DIQMTQSPSTLSASVGDRVTITCRASQSISSWLAWYQQRPGKAPKLLIYKASSLEGGVPSRFSGSGSGTEFTLTISSLQPEDFATYYCQQYNSYWYTFGQGTKLEIK

SEQ ID NO: 347 - DNA, 18 bp, Artificial Sequence
  Synthetic
cagagtattagtagctgg

SEQ ID NO: 348 - protein, 6 aa, Artificial Sequence
  Synthetic
QSISSW

SEQ ID NO: 349 - DNA, 9 bp, Artificial Sequence
  Synthetic
aaggcgtct

SEQ ID NO: 350 - protein, 3 aa, Artificial Sequence
  Synthetic
KAS

SEQ ID NO: 351 - DNA, 27 bp, Artificial Sequence
  Synthetic
caacagtataatagttattggtacact

SEQ ID NO: 352 - protein, 9 aa, Artificial Sequence
  Synthetic
QQYNSYWYT

SEQ ID NO: 353 - DNA, 354 bp, Artificial Sequence; identical to SEQ ID NO: 337
  Synthetic

SEQ ID NO: 354 - protein, 118 aa, Artificial Sequence; identical to SEQ ID NO: 338
  Synthetic

SEQ ID NO: 355 - DNA, 321 bp, Artificial Sequence; identical to SEQ ID NO: 345
  Synthetic

SEQ ID NO: 356 - protein, 107 aa, Artificial Sequence; identical to SEQ ID NO: 346
  Synthetic

SEQ ID NO: 357 - DNA, 354 bp, Artificial Sequence
  Synthetic
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagagagggcagtagcagactttttgactactggggccaaggaaccctggtcaccgtctcctca

SEQ ID NO: 358 - protein, 118 aa, Artificial Sequence
  Synthetic
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCAREGSSRLFDYWGQGTLVTVSS

SEQ ID NO: 359 - DNA, 321 bp, Artificial Sequence
  Synthetic
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttattggtacacttttggccaggggaccaagctggagatcaaa

SEQ ID NO: 360 - protein, 107 aa, Artificial Sequence
  Synthetic
DIQMTQSPSTLSASVGDRVTITCRASQSISSWLAWYQQKPGKAPKLLIYKASSLESGVPSRFSGSGSGTEFTLTISSLQPDDFATYYCQQYNSYWYTFGQGTKLEIK

SEQ ID NO: 361 - DNA, 384 bp, Artificial Sequence
  Synthetic
caggtgcacctggtggagtctgggggaggcttggtcaagcctggagggtccctgagactctcctgtgcagcctctggattcaccttcagtgaccactacatgagctggatccgccaggctccagggaaggggctggagtggatttcatacattagtaatgatggtggtaccaaatactatgtggactctgtggagggccgattcatcatttccagggacaacgccaagaactcattgtatctacatatgaacagcctcagagccgacgacacggccgtgtattactgtgcgagagatcagggatatattggctacgactcgtattattactattcctacggtatggacgtctggggccaagggaccacggtcaccgtcgcctca

SEQ ID NO: 362 - protein, 128 aa, Artificial Sequence
  Synthetic
QVHLVESGGGLVKPGGSLRLSCAASGFTFSDHYMSWIRQAPGKGLEWISYISNDGGTKYYVDSVEGRFIISRDNAKNSLYLHMNSLRADDTAVYYCARDQGYIGYDSYYYYSYGMDVWGQGTTVTVAS

SEQ ID NO: 363 - DNA, 24 bp, Artificial Sequence
  Synthetic
ggattcaccttcagtgaccactac

SEQ ID NO: 364 - protein, 8 aa, Artificial Sequence
  Synthetic
GFTFSDHY

SEQ ID NO: 365 - DNA, 24 bp, Artificial Sequence
  Synthetic
attagtaatgatggtggtaccaaa

SEQ ID NO: 366 - protein, 8 aa, Artificial Sequence
  Synthetic
ISNDGGTK

SEQ ID NO: 367 - DNA, 63 bp, Artificial Sequence
  Synthetic
gcgagagatcagggatatattggctacgactcgtattattactattcctacggtatggacgtc

SEQ ID NO: 368 - protein, 21 aa, Artificial Sequence
  Synthetic
ARDQGYIGYDSYYYYSYGMDV

SEQ ID NO: 369 - DNA, 321 bp, Artificial Sequence
  Synthetic
aaaattgtgttgacgcagtctccaggcaccctgcctttgtttccaggggaaagagccaccctctcctgtagggccagtcagagtgttaacaacaaattcttagcctggtaccagcagaaatctggccaggctcccaggctcctcatctatggtgcatccagcagggccactggcatcccagacaggttcagtggcagtgggtctgggaccgacttcactctcaccatcagcggactggagcctgaagattttgaagtgtattattgtcaagtatatggtaactcactcactctcggcggagggaccaaggtggagatcaag

SEQ ID NO: 370 - protein, 107 aa, Artificial Sequence
  Synthetic
KIVLTQSPGTLPLFPGERATLSCRASQSVNNKFLAWYQQKSGQAPRLLIYGASSRATGIPDRFSGSGSGTDFTLTISGLEPEDFEVYYCQVYGNSLTLGGGTKVEIK

SEQ ID NO: 371 - DNA, 21 bp, Artificial Sequence
  Synthetic
cagagtgttaacaacaaattc

SEQ ID NO: 372 - protein, 7 aa, Artificial Sequence
  Synthetic
QSVNNKF

SEQ ID NO: 373 - DNA, 9 bp, Artificial Sequence
  Synthetic
ggtgcatcc

SEQ ID NO: 374 - protein, 3 aa, Artificial Sequence
  Synthetic
GAS

SEQ ID NO: 375 - DNA, 24 bp, Artificial Sequence
  Synthetic
caagtatatggtaactcactcact

SEQ ID NO: 376 - protein, 8 aa, Artificial Sequence
  Synthetic
QVYGNSLT

SEQ ID NO: 377 - DNA, 384 bp, Artificial Sequence
  Synthetic
caggtgcagctggtggagtctgggggaggcttggtcaagcctggagggtccctgagactctcctgtgcagcctctggattcaccttcagtgaccactacatgagctggatccgccaggctccagggaaggggctggagtggatttcatacattagtaatgatggtggtaccaaatactatgtggactctgtggagggccgattcatcatttccagggacaacgccaagaactcattgtatctacatatgaacagcctcagagccgacgacacggccgtgtattactgtgcgagagatcagggatatattggctacgactcgtattattactattcctacggtatggacgtctggggccaagggaccacggtcaccgtctcctca

SEQ ID NO: 378 - protein, 128 aa, Artificial Sequence
  Synthetic
QVQLVESGGGLVKPGGSLRLSCAASGFTFSDHYMSWIRQAPGKGLEWISYISNDGGTKYYVDSVEGRFIISRDNAKNSLYLHMNSLRADDTAVYYCARDQGYIGYDSYYYYSYGMDVWGQGTTVTVSS

SEQ ID NO: 379 - DNA, 321 bp, Artificial Sequence
  Synthetic
gaaattgtgttgacgcagtctccaggcaccctgcctttgtttccaggggaaagagccaccctctcctgtagggccagtcagagtgttaacaacaaattcttagcctggtaccagcagaaatctggccaggctcccaggctcctcatctatggtgcatccagcagggccactggcatcccagacaggttcagtggcagtgggtctgggaccgacttcactctcaccatcagcggactggagcctgaagattttgaagtgtattattgtcaagtatatggtaactcactcactctcggcggagggaccaaggtggagatcaaa

SEQ ID NO: 380 - protein, 107 aa, Artificial Sequence
  Synthetic
EIVLTQSPGTLPLFPGERATLSCRASQSVNNKFLAWYQQKSGQAPRLLIYGASSRATGIPDRFSGSGSGTDFTLTISGLEPEDFEVYYCQVYGNSLTLGGGTKVEIK

SEQ ID NO: 381 - DNA, 384 bp, Artificial Sequence
  Synthetic
caggtgcagctggtggagtctgggggaggcttggtcaagcctggagggtccctgagactctcctgtgcagcctctggattcaccttcagtgaccactacatgagctggatccgccaggctccagggaaggggctggagtgggtttcatacattagtaatgatggtggtaccaaatactacgcagactctgtgaagggccgattcaccatctccagggacaacgccaagaactcactgtatctgcaaatgaacagcctgagagccgaggacacggccgtgtattactgtgcgagagatcagggatatattggctacgactcgtattattactattcctacggtatggacgtctgggggcaagggaccacggtcaccgtctcctca

SEQ ID NO: 382 - protein, 128 aa, Artificial Sequence
  Synthetic
QVQLVESGGGLVKPGGSLRLSCAASGFTFSDHYMSWIRQAPGKGLEWVSYISNDGGTKYYADSVKGRFTISRDNAKNSLYLQMNSLRAEDTAVYYCARDQGYIGYDSYYYYSYGMDVWGQGTTVTVSS

SEQ ID NO: 383 - DNA, 321 bp, Artificial Sequence
  Synthetic
gaaattgtgttgacgcagtctccaggcaccctgtctttgtctccaggggaaagagccaccctctcctgcagggccagtcagagtgttaacaacaaattcttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcaagtatatggtaactcactcactttcggcggagggaccaaggtggagatcaaa

SEQ ID NO: 384 - protein, 107 aa, Artificial Sequence
  Synthetic
EIVLTQSPGTLSLSPGERATLSCRASQSVNNKFLAWYQQKPGQAPRLLIYGASSRATGIPDRFSGSGSGTDFTLTISRLEPEDFAVYYCQVYGNSLTFGGGTKVEIK

SEQ ID NO: 385 - DNA, 360 bp, Artificial Sequence
  Synthetic
gaggtgcagaaggtggagtctgggggaggcctggtcaagccgggggggtccctgagactctcctgtacagcctctggattcaccttcagtacttataacatgaattgggtccgccaggctccagggaagggactggagtgggtctcatccattaggagtagtagtaattacatatactacgcagactcagtgaagggccgattcaccatctccagagacaacgccaagaattcactgtatctgcaaatgaacagcctgagagccgatgacacggctgtgtattactgtgcgagagatggcagcagttggtacgactactctgactactggggccagggaaccctggtcaccgtctcctca

SEQ ID NO: 386 - protein, 120 aa, Artificial Sequence
  Synthetic
EVQKVESGGGLVKPGGSLRLSCTASGFTFSTYNMNWVRQAPGKGLEWVSSIRSSSNYIYYADSVKGRFTISRDNAKNSLYLQMNSLRADDTAVYYCARDGSSWYDYSDYWGQGTLVTVSS

SEQ ID NO: 387 - DNA, 24 bp, Artificial Sequence
  Synthetic
ggattcaccttcagtacttataac

SEQ ID NO: 388 - protein, 8 aa, Artificial Sequence
  Synthetic
GFTFSTYN

SEQ ID NO: 389 - DNA, 24 bp, Artificial Sequence
  Synthetic
attaggagtagtagtaattacata

SEQ ID NO: 390 - protein, 8 aa, Artificial Sequence
  Synthetic
IRSSSNYI

SEQ ID NO: 391 - DNA, 39 bp, Artificial Sequence
  Synthetic
gcgagagatggcagcagttggtacgactactctgactac

SEQ ID NO: 392 - protein, 13 aa, Artificial Sequence
  Synthetic
ARDGSSWYDYSDY

SEQ ID NO: 393 - DNA, 321 bp, Artificial Sequence
  Synthetic
gacatccagatgacccagtctccttccaccctgtctgcatctgtaggagacagagtcaccatcacttgccgggccagtcagagtattagtagctggttggcctggtatcaacagataccagggaaagcccctaaactcctgatctataaggcgtctagtttagaaaatggggtcccatcaaggttcagcggcagtggatctgggacagaattcactctcatcatcagcagcctgcagcctgatgattttgcaacttattactgccaacagtatattagttattctcggacgttcggccaagggaccaaggtggaaatcaaa

SEQ ID NO: 394 - protein, 107 aa, Artificial Sequence
  Synthetic
DIQMTQSPSTLSASVGDRVTITCRASQSISSWLAWYQQIPGKAPKLLIYKASSLENGVPSRFSGSGSGTEFTLIISSLQPDDFATYYCQQYISYSRTFGQGTKVEIK

SEQ ID NO: 395 - DNA, 18 bp, Artificial Sequence
  Synthetic
cagagtattagtagctgg

SEQ ID NO: 396 - protein, 6 aa, Artificial Sequence
  Synthetic
QSISSW

SEQ ID NO: 397 - DNA, 9 bp, Artificial Sequence
  Synthetic
aaggcgtct

SEQ ID NO: 398 - protein, 3 aa, Artificial Sequence
  Synthetic
KAS

SEQ ID NO: 399 - DNA, 27 bp, Artificial Sequence
  Synthetic
caacagtatattagttattctcggacg

SEQ ID NO: 400 - protein, 9 aa, Artificial Sequence
  Synthetic
QQYISYSRT

SEQ ID NO: 401 - DNA, 360 bp, Artificial Sequence
  Synthetic
gaggtgcagctggtggagtctgggggaggcctggtcaagccgggggggtccctgagactctcctgtacagcctctggattcaccttcagtacttataacatgaattgggtccgccaggctccagggaagggactggagtgggtctcatccattaggagtagtagtaattacatatactacgcagactcagtgaagggccgattcaccatctccagagacaacgccaagaattcactgtatctgcaaatgaacagcctgagagccgatgacacggctgtgtattactgtgcgagagatggcagcagttggtacgactactctgactactggggccagggaaccctggtcaccgtctcctca

SEQ ID NO: 402 - protein, 120 aa, Artificial Sequence
  Synthetic
EVQLVESGGGLVKPGGSLRLSCTASGFTFSTYNMNWVRQAPGKGLEWVSSIRSSSNYIYYADSVKGRFTISRDNAKNSLYLQMNSLRADDTAVYYCARDGSSWYDYSDYWGQGTLVTVSS

SEQ ID NO: 403 - DNA, 321 bp, Artificial Sequence; identical to SEQ ID NO: 393
  Synthetic

SEQ ID NO: 404 - protein, 107 aa, Artificial Sequence; identical to SEQ ID NO: 394
  Synthetic

SEQ ID NO: 405 - DNA, 360 bp, Artificial Sequence
  Synthetic
gaggtgcagctggtggagtctgggggaggcctggtcaagcctggggggtccctgagactctcctgtgcagcctctggattcaccttcagtacttataacatgaactgggtccgccaggctccagggaaggggctggagtgggtctcatccattaggagtagtagtaattacatatactacgcagactcagtgaagggccgattcaccatctccagagacaacgccaagaactcactgtatctgcaaatgaacagcctgagagccgaggacacggctgtgtattactgtgcgagagatggcagcagttggtacgactactctgactactggggccaaggaaccctggtcaccgtctcctca

SEQ ID NO: 406 - protein, 120 aa, Artificial Sequence
  Synthetic
EVQLVESGGGLVKPGGSLRLSCAASGFTFSTYNMNWVRQAPGKGLEWVSSIRSSSNYIYYADSVKGRFTISRDNAKNSLYLQMNSLRAEDTAVYYCARDGSSWYDYSDYWGQGTLVTVSS

SEQ ID NO: 407 - DNA, 321 bp, Artificial Sequence
  Synthetic
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtatattagttattctcggacgttcggccaagggaccaaggtggaaatcaaa

SEQ ID NO: 408 - protein, 107 aa, Artificial Sequence
  Synthetic
DIQMTQSPSTLSASVGDRVTITCRASQSISSWLAWYQQKPGKAPKLLIYKASSLESGVPSRFSGSGSGTEFTLTISSLQPDDFATYYCQQYISYSRTFGQGTKVEIK

SEQ ID NO: 409 - DNA, 360 bp, Artificial Sequence; identical to SEQ ID NO: 401
  Synthetic

SEQ ID NO: 410 - protein, 120 aa, Artificial Sequence; identical to SEQ ID NO: 402
  Synthetic

SEQ ID NO: 411 - DNA, 24 bp, Artificial Sequence
  Synthetic
ggattcaccttcagtacttataac

SEQ ID NO: 412 - protein, 8 aa, Artificial Sequence
  Synthetic
GFTFSTYN

SEQ ID NO: 413 - DNA, 24 bp, Artificial Sequence
  Synthetic
attaggagtagtagtaattacata

SEQ ID NO: 414 - protein, 8 aa, Artificial Sequence
  Synthetic
IRSSSNYI

SEQ ID NO: 415 - DNA, 39 bp, Artificial Sequence; identical to SEQ ID NO: 391
  Synthetic

SEQ ID NO: 416 - protein, 13 aa, Artificial Sequence
  Synthetic
ARDGSSWYDYSDY

SEQ ID NO: 417 - DNA, 321 bp, Artificial Sequence; identical to SEQ ID NO: 393
  Synthetic

SEQ ID NO: 418 - protein, 107 aa, Artificial Sequence; identical to SEQ ID NO: 394
  Synthetic

SEQ ID NO: 419 - DNA, 18 bp, Artificial Sequence
  Synthetic
cagagtattagtagctgg

SEQ ID NO: 420 - protein, 6 aa, Artificial Sequence
  Synthetic
QSISSW

SEQ ID NO: 421 - DNA, 9 bp, Artificial Sequence
  Synthetic
aaggcgtct

SEQ ID NO: 422 - protein, 3 aa, Artificial Sequence
  Synthetic
KAS

SEQ ID NO: 423 - DNA, 27 bp, Artificial Sequence
  Synthetic
caacagtatattagttattctcggacg

SEQ ID NO: 424 - protein, 9 aa, Artificial Sequence
  Synthetic
QQYISYSRT

SEQ ID NO: 425 - DNA, 360 bp, Artificial Sequence; identical to SEQ ID NO: 401
  Synthetic

SEQ ID NO: 426 - protein, 120 aa, Artificial Sequence; identical to SEQ ID NO: 402
  Synthetic

SEQ ID NO: 427 - DNA, 321 bp, Artificial Sequence; identical to SEQ ID NO: 393
  Synthetic

SEQ ID NO: 428 - protein, 107 aa, Artificial Sequence; identical to SEQ ID NO: 394
  Synthetic

SEQ ID NO: 429 - DNA, 360 bp, Artificial Sequence; identical to SEQ ID NO: 405
  Synthetic

SEQ ID NO: 430 - protein, 120 aa, Artificial Sequence; identical to SEQ ID NO: 406
  Synthetic

SEQ ID NO: 431 - DNA, 321 bp, Artificial Sequence; identical to SEQ ID NO: 407
  Synthetic

SEQ ID NO: 432 - protein, 107 aa, Artificial Sequence; identical to SEQ ID NO: 408
  Synthetic

SEQ ID NO: 433 - DNA, 360 bp, Artificial Sequence
  Synthetic
gaggtgcagctggtggagtctgggggaggcctggtcaagccgggggggtccctgagactctcctgtacagcctctggattcaccttcagtacttataacatgaattgggtccgccaggctccagggaagggactggagtgggtctcatccattaggagtagtagtaattacatatactacgcagactcagtgaagggccgattcaccatctccagagacaacgccaagagttcactgtatctgcaaatgaacagcctgagagccgaggacacggctgtgtattactgtgcgagagatggcagcagttggtacgactactctgactactggggccagggaaccctggtcaccgtctcctca

SEQ ID NO: 434 - protein, 120 aa, Artificial Sequence
  Synthetic
EVQLVESGGGLVKPGGSLRLSCTASGFTFSTYNMNWVRQAPGKGLEWVSSIRSSSNYIYYADSVKGRFTISRDNAKSSLYLQMNSLRAEDTAVYYCARDGSSWYDYSDYWGQGTLVTVSS

SEQ ID NO: 435 - DNA, 24 bp, Artificial Sequence
  Synthetic
ggattcaccttcagtacttataac

SEQ ID NO: 436 - protein, 8 aa, Artificial Sequence
  Synthetic
GFTFSTYN

SEQ ID NO: 437 - DNA, 24 bp, Artificial Sequence
  Synthetic
attaggagtagtagtaattacata

SEQ ID NO: 438 - protein, 8 aa, Artificial Sequence
  Synthetic
IRSSSNYI

SEQ ID NO: 439 - DNA, 39 bp, Artificial Sequence; identical to SEQ ID NO: 391
  Synthetic

SEQ ID NO: 440 - protein, 13 aa, Artificial Sequence
  Synthetic
ARDGSSWYDYSDY

SEQ ID NO: 441 - DNA, 321 bp, Artificial Sequence
  Synthetic
gacatccagatgacccagtctccttccaccctgtctgcatctgtaggagacagagtcaccatcacttgccgggccagtcagagtattagtagctggttggcctggtatcaacaggtaccagggaaagcccctaaactcctgatctataaggcgtctagtttagaaaatggggtcccatcaaggttcagcggcagtggatctgggacagaattcactctcatcatcagcagcctgcagcctgatgattttgcaacttattactgccaacagtatattagttattctcggacgttcggccaagggaccaaggtggaaatcaaa

SEQ ID NO: 442 - protein, 107 aa, Artificial Sequence
  Synthetic
DIQMTQSPSTLSASVGDRVTITCRASQSISSWLAWYQQVPGKAPKLLIYKASSLENGVPSRFSGSGSGTEFTLIISSLQPDDFATYYCQQYISYSRTFGQGTKVEIK

SEQ ID NO: 443 - DNA, 18 bp, Artificial Sequence
  Synthetic
cagagtattagtagctgg

SEQ ID NO: 444 - protein, 6 aa, Artificial Sequence
  Synthetic
QSISSW

SEQ ID NO: 445 - DNA, 9 bp, Artificial Sequence
  Synthetic
aaggcgtct

SEQ ID NO: 446 - protein, 3 aa, Artificial Sequence
  Synthetic
KAS

SEQ ID NO: 447 - DNA, 27 bp, Artificial Sequence
  Synthetic
caacagtatattagttattctcggacg

SEQ ID NO: 448 - protein, 9 aa, Artificial Sequence
  Synthetic
QQYISYSRT

SEQ ID NO: 449 - DNA, 360 bp, Artificial Sequence; identical to SEQ ID NO: 433
  Synthetic

SEQ ID NO: 450 - protein, 120 aa, Artificial Sequence; identical to SEQ ID NO: 434
  Synthetic

SEQ ID NO: 451 - DNA, 321 bp, Artificial Sequence; identical to SEQ ID NO: 441
  Synthetic

SEQ ID NO: 452 - protein, 107 aa, Artificial Sequence; identical to SEQ ID NO: 442
  Synthetic

SEQ ID NO: 453 - DNA, 360 bp, Artificial Sequence
  Synthetic
gaggtgcagctggtggagtctgggggaggcctggtcaagcctggggggtccctgagactctcctgtgcagcctctggattcaccttcagtacttataacatgaactgggtccgccaggctccagggaaggggctggagtgggtctcatccattaggagtagtagtaattacatatactacgcagactcagtgaagggccgattcaccatctccagagacaacgccaagaactcactgtatctgcaaatgaacagcctgagagccgaggacacggctgtgtattactgtgcgagagatggcagcagttggtacgactactctgactactggggccagggaaccctggtcaccgtctcctca

SEQ ID NO: 454 - protein, 120 aa, Artificial Sequence; identical to SEQ ID NO: 406
  Synthetic

SEQ ID NO: 455 - DNA, 321 bp, Artificial Sequence; identical to SEQ ID NO: 407
  Synthetic

SEQ ID NO: 456 - protein, 107 aa, Artificial Sequence; identical to SEQ ID NO: 408
  Synthetic

SEQ ID NO: 457 - DNA, 360 bp, Artificial Sequence; identical to SEQ ID NO: 401
  Synthetic

SEQ ID NO: 458 - protein, 120 aa, Artificial Sequence; identical to SEQ ID NO: 402
  Synthetic

SEQ ID NO: 459 - DNA, 24 bp, Artificial Sequence
  Synthetic
ggattcaccttcagtacttataac

SEQ ID NO: 460 - protein, 8 aa, Artificial Sequence
  Synthetic
GFTFSTYN

SEQ ID NO: 461 - DNA, 24 bp, Artificial Sequence
  Synthetic
attaggagtagtagtaattacata

SEQ ID NO: 462 - protein, 8 aa, Artificial Sequence
  Synthetic
IRSSSNYI

SEQ ID NO: 463 - DNA, 39 bp, Artificial Sequence; identical to SEQ ID NO: 391
  Synthetic

SEQ ID NO: 464 - protein, 13 aa, Artificial Sequence
  Synthetic
ARDGSSWYDYSDY

SEQ ID NO: 465 - DNA, 321 bp, Artificial Sequence; identical to SEQ ID NO: 393
  Synthetic

SEQ ID NO: 466 - protein, 107 aa, Artificial Sequence; identical to SEQ ID NO: 394
  Synthetic

SEQ ID NO: 467 - DNA, 18 bp, Artificial Sequence
  Synthetic
cagagtattagtagctgg

SEQ ID NO: 468 - protein, 6 aa, Artificial Sequence
  Synthetic
QSISSW

SEQ ID NO: 469 - DNA, 9 bp, Artificial Sequence
  Synthetic
aaggcgtct

SEQ ID NO: 470 - protein, 3 aa, Artificial Sequence
  Synthetic
KAS

SEQ ID NO: 471 - DNA, 27 bp, Artificial Sequence
  Synthetic
caacagtatattagttattctcggacg

SEQ ID NO: 472 - protein, 9 aa, Artificial Sequence
  Synthetic
QQYISYSRT

SEQ ID NO: 473 - DNA, 360 bp, Artificial Sequence; identical to SEQ ID NO: 401
  Synthetic

SEQ ID NO: 474 - protein, 120 aa, Artificial Sequence; identical to SEQ ID NO: 402
  Synthetic

SEQ ID NO: 475 - DNA, 321 bp, Artificial Sequence; identical to SEQ ID NO: 393
  Synthetic

SEQ ID NO: 476 - protein, 107 aa, Artificial Sequence; identical to SEQ ID NO: 394
  Synthetic

SEQ ID NO: 477 - DNA, 360 bp, Artificial Sequence; identical to SEQ ID NO: 405
  Synthetic

SEQ ID NO: 478 - protein, 120 aa, Artificial Sequence; identical to SEQ ID NO: 406
  Synthetic

SEQ ID NO: 479 - DNA, 321 bp, Artificial Sequence; identical to SEQ ID NO: 407
  Synthetic

SEQ ID NO: 480 - protein, 107 aa, Artificial Sequence; identical to SEQ ID NO: 408
  Synthetic

SEQ ID NO: 481 - DNA, 354 bp, Artificial Sequence
  Synthetic
gaggtgcaactagtggagtctgggggaggcttggtacagcctggggggtccctgagactctcctgtgtagtctctggattcaccttcggtgactacgacatgcactgggtccgtcaagctacaggaagaggtctggagtgggtctcaggtattgctcctgctggtgacacatcctatacaggctccgtgaagggccgattcaccatctccagagagaatgccaagaactccttgcatcttcaaatgaacagcctgacaaccggggacacggctatatattattgtgctagagaggatatagcagtgcctggttttgattactggggccagggaaccctggtcaccgtctcctca

SEQ ID NO: 482 - protein, 118 aa, Artificial Sequence
  Synthetic
EVQLVESGGGLVQPGGSLRLSCVVSGFTFGDYDMHWVRQATGRGLEWVSGIAPAGDTSYTGSVKGRFTISRENAKNSLHLQMNSLTTGDTAIYYCAREDIAVPGFDYWGQGTLVTVSS

SEQ ID NO: 483 - DNA, 24 bp, Artificial Sequence
  Synthetic
ggattcaccttcggtgactacgac

SEQ ID NO: 484 - protein, 8 aa, Artificial Sequence
  Synthetic
GFTFGDYD

SEQ ID NO: 485 - DNA, 21 bp, Artificial Sequence
  Synthetic
attgctcctgctggtgacaca

SEQ ID NO: 486 - protein, 7 aa, Artificial Sequence
  Synthetic
IAPAGDT

SEQ ID NO: 487 - DNA, 36 bp, Artificial Sequence
  Synthetic
gctagagaggatatagcagtgcctggttttgattac

SEQ ID NO: 488 - protein, 12 aa, Artificial Sequence
  Synthetic
AREDIAVPGFDY

SEQ ID NO: 489 - DNA, 324 bp, Artificial Sequence
  Synthetic
gaaatagtgatgacgcagtctccagccaccctgtctgtgtctccaggggaacgaggcaccctctcctgcagggccagtcagagtgttagcagcaacttagcctggtaccagcagaaacctggccaggctcccagactcctcatctatggtgcatccacgagggccactggcttcccagccaggttcagtggcagtgggtctgggacagagttcactctcaccatcagcagcctgcagtctgaagattttgcagtttattactgtcagcagtataataagtggcctccgttcactttcggccctgggaccaaagtggatttcaaa

SEQ ID NO: 490 - protein, 108 aa, Artificial Sequence
  Synthetic
EIVMTQSPATLSVSPGERGTLSCRASQSVSSNLAWYQQKPGQAPRLLIYGASTRATGFPARFSGSGSGTEFTLTISSLQSEDFAVYYCQQYNKWPPFTFGPGTKVDFK

SEQ ID NO: 491 - DNA, 18 bp, Artificial Sequence
  Synthetic
cagagtgttagcagcaac

SEQ ID NO: 492 - protein, 6 aa, Artificial Sequence
  Synthetic
QSVSSN

SEQ ID NO: 493 - DNA, 9 bp, Artificial Sequence
  Synthetic
ggtgcatcc

SEQ ID NO: 494 - protein, 3 aa, Artificial Sequence
  Synthetic
GAS

SEQ ID NO: 495 - DNA, 30 bp, Artificial Sequence
  Synthetic
cagcagtataataagtggcctccgttcact

SEQ ID NO: 496 - protein, 10 aa, Artificial Sequence
  Synthetic
QQYNKWPPFT

SEQ ID NO: 497 - DNA, 354 bp, Artificial Sequence; identical to SEQ ID NO: 481
  Synthetic

SEQ ID NO: 498 - protein, 118 aa, Artificial Sequence; identical to SEQ ID NO: 482
  Synthetic

SEQ ID NO: 499 - DNA, 324 bp, Artificial Sequence
  Synthetic
gaaatagtgatgacgcagtctccagccaccctgtctgtgtctccaggggaacgaggcaccctctcctgcagggccagtcagagtgttagcagcaacttagcctggtaccagcagaaacctggccaggctcccagactcctcatctatggtgcatccacgagggccactggcttcccagccaggttcagtggcagtgggtctgggacagagttcactctcaccatcagcagcctgcagtctgaagattttgcagtttattactgtcagcagtataataagtggcctccgttcactttcggccctgggaccaaagtggatatcaaa

SEQ ID NO: 500 - protein, 108 aa, Artificial Sequence
  Synthetic
EIVMTQSPATLSVSPGERGTLSCRASQSVSSNLAWYQQKPGQAPRLLIYGASTRATGFPARFSGSGSGTEFTLTISSLQSEDFAVYYCQQYNKWPPFTFGPGTKVDIK

SEQ ID NO: 501 - DNA, 354 bp, Artificial Sequence
  Synthetic
gaggtgcagctggtggagtctgggggaggcttggtacagcctggggggtccctgagactctcctgtgcagcctctggattcaccttcggtgactacgacatgcactgggtccgccaagctacaggaaaaggtctggagtgggtctcagctattgctcctgctggtgacacatactatccaggctccgtgaagggccgattcaccatctccagagaaaatgccaagaactccttgtatcttcaaatgaacagcctgagagccggggacacggctgtgtattactgtgctagagaggatatagcagtgcctggttttgattactggggccaaggaaccctggtcaccgtctcctca

SEQ ID NO: 502 - protein, 118 aa, Artificial Sequence
  Synthetic
EVQLVESGGGLVQPGGSLRLSCAASGFTFGDYDMHWVRQATGKGLEWVSAIAPAGDTYYPGSVKGRFTISRENAKNSLYLQMNSLRAGDTAVYYCAREDIAVPGFDYWGQGTLVTVSS

SEQ ID NO: 503 - DNA, 324 bp, Artificial Sequence
  Synthetic
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataagtggcctccgttcactttcggccctgggaccaaagtggatatcaaa

SEQ ID NO: 504 - protein, 108 aa, Artificial Sequence
  Synthetic
EIVMTQSPATLSVSPGERATLSCRASQSVSSNLAWYQQKPGQAPRLLIYGASTRATGIPARFSGSGSGTEFTLTISSLQSEDFAVYYCQQYNKWPPFTFGPGTKVDIK

SEQ ID NO: 505 - DNA, 378 bp, Artificial Sequence
  Synthetic
caaattctgctggtgcaatctggacctgaggtgaaggagcctggggcctcagtgaaggtctcctgcaaggcttctggttacacctttaccaactacgctatcagctgggtgcgacaggtccctggacaagggcttgagtggatgggatgggtcagcgcttacaatggtcacacaaactatgcacatgaagtccagggcagagtcaccatgaccacagacacatccacgaccacagcctacatggagctgaggagcctgagatctgacgacacggccatgtattactgtgcgagagggggtgtagtcgtgccagttgctccccacttctacaacggtatggacgtctggggccaagggaccacggtcaccgtctcctca

SEQ ID NO: 506 - protein, 126 aa, Artificial Sequence
  Synthetic
QILLVQSGPEVKEPGASVKVSCKASGYTFTNYAISWVRQVPGQGLEWMGWVSAYNGHTNYAHEVQGRVTMTTDTSTTTAYMELRSLRSDDTAMYYCARGGVVVPVAPHFYNGMDVWGQGTTVTVSS

SEQ ID NO: 507 - DNA, 24 bp, Artificial Sequence
  Synthetic
ggttacacctttaccaactacgct

SEQ ID NO: 508 - protein, 8 aa, Artificial Sequence
  Synthetic
GYTFTNYA

SEQ ID NO: 509 - DNA, 24 bp, Artificial Sequence
  Synthetic
gtcagcgcttacaatggtcacaca

SEQ ID NO: 510 - protein, 8 aa, Artificial Sequence
  Synthetic
VSAYNGHT

SEQ ID NO: 511 - DNA, 57 bp, Artificial Sequence
  Synthetic
gcgagagggggtgtagtcgtgccagttgctccccacttctacaacggtatggacgtc

SEQ ID NO: 512 - protein, 19 aa, Artificial Sequence
  Synthetic
ARGGVVVPVAPHFYNGMDV

SEQ ID NO: 513 - DNA, 336 bp, Artificial Sequence
  Synthetic
gatattgtgatgactcagtttccactctccctgcccgtcacccctggagagccggcctccatctcctgcaggtctagtcagagcctcctgcatattaatgaatacaactatttggattggtacctaaagaagccagggcagtctccacagctcctgatctatttgggttttaatcgggcctccggggtccctgacaggttcagtggcagtggatcaggcacagattttacactgaaaatcagcagagtggaggctgaggatgttggggtctattactgcatgcaagctcttcaaactccgtggacgttaggccaagggaccaaggtggaaatcaaa

SEQ ID NO: 514 - protein, 112 aa, Artificial Sequence
  Synthetic
DIVMTQFPLSLPVTPGEPASISCRSSQSLLHINEYNYLDWYLKKPGQSPQLLIYLGFNRASGVPDRFSGSGSGTDFTLKISRVEAEDVGVYYCMQALQTPWTLGQGTKVEIK

SEQ ID NO: 515 - DNA, 33 bp, Artificial Sequence
  Synthetic
cagagcctcctgcatattaatgaatacaactat

SEQ ID NO: 516 - protein, 11 aa, Artificial Sequence
  Synthetic
QSLLHINEYNY

SEQ ID NO: 517 - DNA, 9 bp, Artificial Sequence
  Synthetic
ttgggtttt

SEQ ID NO: 518 - protein, 3 aa, Artificial Sequence
  Synthetic
LGF

SEQ ID NO: 519 - DNA, 27 bp, Artificial Sequence
  Synthetic
atgcaagctcttcaaactccgtggacg

SEQ ID NO: 520 - protein, 9 aa, Artificial Sequence
  Synthetic
MQALQTPWT

SEQ ID NO: 521 - DNA, 378 bp, Artificial Sequence
  Synthetic
caggttcagctggtgcagtctggacctgaggtgaaggagcctggggcctcagtgaaggtctcctgcaaggcttctggttacacctttaccaactacgctatcagctgggtgcgacaggtccctggacaagggcttgagtggatgggatgggtcagcgcttacaatggtcacacaaactatgcacatgaagtccagggcagagtcaccatgaccacagacacatccacgaccacagcctacatggagctgaggagcctgagatctgacgacacggccatgtattactgtgcgagagggggtgtagtcgtgccagttgctccccacttctacaacggtatggacgtctggggccaagggaccacggtcaccgtctcctca

SEQ ID NO: 522 - protein, 126 aa, Artificial Sequence
  Synthetic
QVQLVQSGPEVKEPGASVKVSCKASGYTFTNYAISWVRQVPGQGLEWMGWVSAYNGHTNYAHEVQGRVTMTTDTSTTTAYMELRSLRSDDTAMYYCARGGVVVPVAPHFYNGMDVWGQGTTVTVSS

SEQ ID NO: 523 - DNA, 336 bp, Artificial Sequence
  Synthetic
gatattgtgatgactcagtctccactctccctgcccgtcacccctggagagccggcctccatctcctgcaggtctagtcagagcctcctgcatattaatgaatacaactatttggattggtacctaaagaagccagggcagtctccacagctcctgatctatttgggttttaatcgggcctccggggtccctgacaggttcagtggcagtggatcaggcacagattttacactgaaaatcagcagagtggaggctgaggatgttggggtctattactgcatgcaagctcttcaaactccgtggacgttaggccaagggaccaaggtggaaatcaaa

SEQ ID NO: 524 - protein, 112 aa, Artificial Sequence
  Synthetic
DIVMTQSPLSLPVTPGEPASISCRSSQSLLHINEYNYLDWYLKKPGQSPQLLIYLGFNRASGVPDRFSGSGSGTDFTLKISRVEAEDVGVYYCMQALQTPWTLGQGTKVEIK

SEQ ID NO: 525 - DNA, 378 bp, Artificial Sequence
  Synthetic
caggttcagctggtgcagtctggagctgaggtgaagaagcctggggcctcagtgaaggtctcctgcaaggcttctggttacacctttaccaactacgctatcagctgggtgcgacaggcccctggacaagggcttgagtggatgggatgggtcagcgcttacaatggtcacacaaactatgcacagaagctccagggcagagtcaccatgaccacagacacatccacgagcacagcctacatggagctgaggagcctgagatctgacgacacggccgtgtattactgtgcgagagggggtgtagtcgtgccagttgctccccacttctacaacggtatggacgtctgggggcaagggaccacggtcaccgtctcctca

SEQ ID NO: 526 - protein, 126 aa, Artificial Sequence
  Synthetic
QVQLVQSGAEVKKPGASVKVSCKASGYTFTNYAISWVRQAPGQGLEWMGWVSAYNGHTNYAQKLQGRVTMTTDTSTSTAYMELRSLRSDDTAVYYCARGGVVVPVAPHFYNGMDVWGQGTTVTVSS

SEQ ID NO: 527 - DNA, 336 bp, Artificial Sequence
  Synthetic
gatattgtgatgactcagtctccactctccctgcccgtcacccctggagagccggcctccatctcctgcaggtctagtcagagcctcctgcatattaatgaatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctcttcaaactccgtggacgttcggccaagggaccaaggtggaaatcaaa

SEQ ID NO: 528 - protein, 112 aa, Artificial Sequence
  Synthetic
DIVMTQSPLSLPVTPGEPASISCRSSQSLLHINEYNYLDWYLQKPGQSPQLLIYLGSNRASGVPDRFSGSGSGTDFTLKISRVEAEDVGVYYCMQALQTPWTFGQGTKVEIK

SEQ ID NO: 529 - DNA, 351 bp, Artificial Sequence
  Synthetic
gaggtgcagctggtggagtctgggggaggcttggtacagcctggggggtccctgagactctcctgtgcagcctctggattcaccctaagtagctacgacatgcactgggtccgccaagcaacaggaaaaggtctggagtgggtctcagctattggcagtactggtgacacatactatacaggctccgtgatgggccgattcaccatctccagagacgctgccaaaaactccttctatcttgaaatgaacagcctgagagtcggggacacggctgtatattactgtgcaagagagggaataagaacaccctatgattattggggccagggagcccgggtcaccgtctcctca

SEQ ID NO: 530 - protein, 117 aa, Artificial Sequence
  Synthetic
EVQLVESGGGLVQPGGSLRLSCAASGFTLSSYDMHWVRQATGKGLEWVSAIGSTGDTYYTGSVMGRFTISRDAAKNSFYLEMNSLRVGDTAVYYCAREGIRTPYDYWGQGARVTVSS

SEQ ID NO: 531 - DNA, 24 bp, Artificial Sequence
  Synthetic
ggattcaccctaagtagctacgac

SEQ ID NO: 532 - protein, 8 aa, Artificial Sequence
  Synthetic
GFTLSSYD

SEQ ID NO: 533 - DNA, 21 bp, Artificial Sequence
  Synthetic
attggcagtactggtgacaca

SEQ ID NO: 534 - protein, 7 aa, Artificial Sequence
  Synthetic
IGSTGDT

SEQ ID NO: 535 - DNA, 33 bp, Artificial Sequence
  Synthetic
gcaagagagggaataagaacaccctatgattat

SEQ ID NO: 536 - protein, 11 aa, Artificial Sequence
  Synthetic
AREGIRTPYDY

SEQ ID NO: 537 - DNA, 324 bp, Artificial Sequence
  Synthetic
gaaatagtgatgacgcagtctccagccaccctgtctgtgtctccaggggaaagagccaccctctcctgcagggccagtcagagtgttagcagcaatgtagcctggtaccagcagaaacctggccaggctcccaggctcctcatctatggtgcatccaccagggccactggtatcccagccaggttcagtggcagtgggtctgggacagaattcactctcaccatcagcagcctgcagtctgaagattttgcagtttattactgtcagcagtataataattggcctccattcactttcggccctgggaccaaagtggatatcaaa

SEQ ID NO: 538 - protein, 108 aa, Artificial Sequence
  Synthetic
EIVMTQSPATLSVSPGERATLSCRASQSVSSNVAWYQQKPGQAPRLLIYGASTRATGIPARFSGSGSGTEFTLTISSLQSEDFAVYYCQQYNNWPPFTFGPGTKVDIK

SEQ ID NO: 539 - DNA, 18 bp, Artificial Sequence
  Synthetic
cagagtgttagcagcaat

SEQ ID NO: 540 - protein, 6 aa, Artificial Sequence
  Synthetic
QSVSSN

SEQ ID NO: 541 - DNA, 9 bp, Artificial Sequence
  Synthetic
ggtgcatcc

SEQ ID NO: 542 - protein, 3 aa, Artificial Sequence
  Synthetic
GAS

SEQ ID NO: 543 - DNA, 30 bp, Artificial Sequence
  Synthetic
cagcagtataataattggcctccattcact

SEQ ID NO: 544 - protein, 10 aa, Artificial Sequence
  Synthetic
QQYNNWPPFT

SEQ ID NO: 545 - DNA, 351 bp, Artificial Sequence
  Synthetic
gaggtgcagctggtggagtctgggggaggcttggtacagcctggggggtccctgagactctcctgtgcagcctctggattcaccctaagtagctacgacatgcactgggtccgccaagcaacaggaaaaggtctggagtgggtctcagctattggcagtactggtgacacatactatacaggctccgtgatgggccgattcaccatctccagagacgctgccaaaaactccttctatcttgaaatgaacagcctgagagtcggggacacggctgtatattactgtgcaagagagggaataagaacaccctatgattattggggccagggaaccctggtcaccgtctcctca

SEQ ID NO: 546 - protein, 117 aa, Artificial Sequence
  Synthetic
EVQLVESGGGLVQPGGSLRLSCAASGFTLSSYDMHWVRQATGKGLEWVSAIGSTGDTYYTGSVMGRFTISRDAAKNSFYLEMNSLRVGDTAVYYCAREGIRTPYDYWGQGTLVTVSS

SEQ ID NO: 547 - DNA, 324 bp, Artificial Sequence; identical to SEQ ID NO: 537
  Synthetic

SEQ ID NO: 548 - protein, 108 aa, Artificial Sequence; identical to SEQ ID NO: 538
  Synthetic

SEQ ID NO: 549 - DNA, 351 bp, Artificial Sequence
  Synthetic
gaggtgcagctggtggagtctgggggaggcttggtacagcctggggggtccctgagactctcctgtgcagcctctggattcaccctaagtagctacgacatgcactgggtccgccaagctacaggaaaaggtctggagtgggtctcagctattggcagtactggtgacacatactatccaggctccgtgaagggccgattcaccatctccagagaaaatgccaagaactccttgtatcttcaaatgaacagcctgagagccggggacacggctgtgtattactgtgcaagagagggaataagaacaccctatgattattggggccaaggaaccctggtcaccgtctcctca

SEQ ID NO: 550 - protein, 117 aa, Artificial Sequence
  Synthetic
EVQLVESGGGLVQPGGSLRLSCAASGFTLSSYDMHWVRQATGKGLEWVSAIGSTGDTYYPGSVKGRFTISRENAKNSLYLQMNSLRAGDTAVYYCAREGIRTPYDYWGQGTLVTVSS

SEQ ID NO: 551 - DNA, 324 bp, Artificial Sequence
  Synthetic
gaaatagtgatgacgcagtctccagccaccctgtctgtgtctccaggggaaagagccaccctctcctgcagggccagtcagagtgttagcagcaatttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataattggcctccattcactttcggccctgggaccaaagtggatatcaaa

SEQ ID NO: 552 - protein, 108 aa, Artificial Sequence; identical to SEQ ID NO: 24
  Synthetic

SEQ ID NO: 553 - DNA, 351 bp, Artificial Sequence; identical to SEQ ID NO: 529
  Synthetic

SEQ ID NO: 554 - protein, 117 aa, Artificial Sequence; identical to SEQ ID NO: 530
  Synthetic

SEQ ID NO: 555 - DNA, 24 bp, Artificial Sequence
  Synthetic
ggattcaccctaagtagctacgac

SEQ ID NO: 556 - protein, 8 aa, Artificial Sequence
  Synthetic
GFTLSSYD

SEQ ID NO: 557 - DNA, 21 bp, Artificial Sequence
  Synthetic
attggcagtactggtgacaca

SEQ ID NO: 558 - protein, 7 aa, Artificial Sequence
  Synthetic
IGSTGDT

SEQ ID NO: 559 - DNA, 33 bp, Artificial Sequence; identical to SEQ ID NO: 535
  Synthetic

SEQ ID NO: 560 - protein, 11 aa, Artificial Sequence
  Synthetic
AREGIRTPYDY

SEQ ID NO: 561 - DNA, 324 bp, Artificial Sequence; identical to SEQ ID NO: 537
  Synthetic

SEQ ID NO: 562 - protein, 108 aa, Artificial Sequence; identical to SEQ ID NO: 538
  Synthetic

SEQ ID NO: 563 - DNA, 18 bp, Artificial Sequence
  Synthetic
cagagtgttagcagcaat

SEQ ID NO: 564 - protein, 6 aa, Artificial Sequence
  Synthetic
QSVSSN

SEQ ID NO: 565 - DNA, 9 bp, Artificial Sequence
  Synthetic
ggtgcatcc

SEQ ID NO: 566 - protein, 3 aa, Artificial Sequence
  Synthetic
GAS

SEQ ID NO: 567 - DNA, 30 bp, Artificial Sequence; identical to SEQ ID NO: 543
  Synthetic

SEQ ID NO: 568 - protein, 10 aa, Artificial Sequence
  Synthetic
QQYNNWPPFT

SEQ ID NO: 569 - DNA, 351 bp, Artificial Sequence; identical to SEQ ID NO: 545
  Synthetic

SEQ ID NO: 570 - protein, 117 aa, Artificial Sequence; identical to SEQ ID NO: 546
  Synthetic

SEQ ID NO: 571 - DNA, 324 bp, Artificial Sequence; identical to SEQ ID NO: 537
  Synthetic

SEQ ID NO: 572 - protein, 108 aa, Artificial Sequence; identical to SEQ ID NO: 538
  Synthetic

SEQ ID NO: 573 - DNA, 351 bp, Artificial Sequence; identical to SEQ ID NO: 549
  Synthetic

SEQ ID NO: 574 - protein, 117 aa, Artificial Sequence; identical to SEQ ID NO: 550
  Synthetic

SEQ ID NO: 575 - DNA, 324 bp, Artificial Sequence; identical to SEQ ID NO: 551
  Synthetic

SEQ ID NO: 576 - protein, 108 aa, Artificial Sequence; identical to SEQ ID NO: 24
  Synthetic

SEQ ID NO: 577 - DNA, 363 bp, Artificial Sequence
  Synthetic
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattaattggaacagtggtagcataggctatgcggactctgtgaagggccgattcaccatctccagagacaacgccaagcactccctgtatctgcaaatgaacagtctgagacctgaggacacggccttgtattactgtgtaaaagaggtgactacgggatactactacggtatggacgtctggggccaagggaccacggtcaccgtctcctca

SEQ ID NO: 578 - protein, 121 aa, Artificial Sequence
  Synthetic
EVQLVESGGGLVQPGRSLRLSCAASGFTFDDYAMHWVRQAPGKGLEWVSGINWNSGSIGYADSVKGRFTISRDNAKHSLYLQMNSLRPEDTALYYCVKEVTTGYYYGMDVWGQGTTVTVSS

SEQ ID NO: 579 - DNA, 24 bp, Artificial Sequence
  Synthetic
ggattcacctttgatgattatgcc

SEQ ID NO: 580 - protein, 8 aa, Artificial Sequence
  Synthetic
GFTFDDYA

SEQ ID NO: 581 - DNA, 24 bp, Artificial Sequence
  Synthetic
attaattggaacagtggtagcata

SEQ ID NO: 582 - protein, 8 aa, Artificial Sequence
  Synthetic
INWNSGSI

SEQ ID NO: 583 - DNA, 42 bp, Artificial Sequence
  Synthetic
gtaaaagaggtgactacgggatactactacggtatggacgtc

SEQ ID NO: 584 - protein, 14 aa, Artificial Sequence
  Synthetic
VKEVTTGYYYGMDV

SEQ ID NO: 585 - DNA, 321 bp, Artificial Sequence
  Synthetic
gacatccagttgacccagtctccatccttcctgtctgcatctgtaggagacagagtcaccatcacttgctgggccagtcagggcattagcagttatttagcctggtatcagaaaaaaccagggaaagcccctaacctcctgatctatgatgcatccactttgcaaagtggggtcccatcaaggttcagcggcagtggatctgggacagaattcactctcacactcagcagcctgcagcctgaagattttgcaacttattactgtcaacagcttaatatttacccattcactttcggccctgggaccaaagtggatatcaaa

SEQ ID NO: 586 - protein, 107 aa, Artificial Sequence
  Synthetic
DIQLTQSPSFLSASVGDRVTITCWASQGISSYLAWYQKKPGKAPNLLIYDASTLQSGVPSRFSGSGSGTEFTLTLSSLQPEDFATYYCQQLNIYPFTFGPGTKVDIK

SEQ ID NO: 587 - DNA, 18 bp, Artificial Sequence
  Synthetic
cagggcattagcagttat

SEQ ID NO: 588 - protein, 6 aa, Artificial Sequence
  Synthetic
QGISSY

SEQ ID NO: 589 - DNA, 9 bp, Artificial Sequence
  Synthetic
gatgcatcc

SEQ ID NO: 590 - protein, 3 aa, Artificial Sequence
  Synthetic
DAS

SEQ ID NO: 591 - DNA, 27 bp, Artificial Sequence
  Synthetic
caacagcttaatatttacccattcact

SEQ ID NO: 592 - protein, 9 aa, Artificial Sequence
  Synthetic
QQLNIYPFT

SEQ ID NO: 593 - DNA, 363 bp, Artificial Sequence; identical to SEQ ID NO: 577
  Synthetic

SEQ ID NO: 594 - protein, 121 aa, Artificial Sequence; identical to SEQ ID NO: 578
  Synthetic

SEQ ID NO: 595 - DNA, 321 bp, Artificial Sequence; identical to SEQ ID NO: 585
  Synthetic

SEQ ID NO: 596 - protein, 107 aa, Artificial Sequence; identical to SEQ ID NO: 586
  Synthetic

SEQ ID NO: 597 - DNA, 363 bp, Artificial Sequence
  Synthetic
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattaattggaacagtggtagcataggctatgcggactctgtgaagggccgattcaccatctccagagacaacgccaagaactccctgtatctgcaaatgaacagtctgagagctgaggacacggccttgtattactgtgtaaaagaggtgactacgggatactactacggtatggacgtctgggggcaagggaccacggtcaccgtctcctca

SEQ ID NO: 598 - protein, 121 aa, Artificial Sequence
  Synthetic
EVQLVESGGGLVQPGRSLRLSCAASGFTFDDYAMHWVRQAPGKGLEWVSGINWNSGSIGYADSVKGRFTISRDNAKNSLYLQMNSLRAEDTALYYCVKEVTTGYYYGMDVWGQGTTVTVSS

SEQ ID NO: 599 - DNA, 321 bp, Artificial Sequence
  Synthetic
gacatccagttgacccagtctccatccttcctgtctgcatctgtaggagacagagtcaccatcacttgccgggccagtcagggcattagcagttatttagcctggtatcagcaaaaaccagggaaagcccctaagctcctgatctatgatgcatccactttgcaaagtggggtcccatcaaggttcagcggcagtggatctgggacagaattcactctcacaatcagcagcctgcagcctgaagattttgcaacttattactgtcaacagcttaatatttacccattcactttcggccctgggaccaaagtggatatcaaa

SEQ ID NO: 600 - protein, 107 aa, Artificial Sequence
  Synthetic
DIQLTQSPSFLSASVGDRVTITCRASQGISSYLAWYQQKPGKAPKLLIYDASTLQSGVPSRFSGSGSGTEFTLTISSLQPEDFATYYCQQLNIYPFTFGPGTKVDIK

SEQ ID NO: 601 - DNA, 366 bp, Artificial Sequence
  Synthetic
gaggtgcagttgttggagtctgggggaggcttggtacagcctggggggtccctgagactctcctgtgcagcctctggattcacgtttagtagctatgccatgaactgggtccgccaggctccagggaaggggctggattgggtctcaggtatcagtggtaatggtggtagcacctactacgcagactccgtgaagggccggttcaccatctccagagacatttccaagaacacgctgtatgtgcaaatgcacagcctgagagtcgaggacacggccgtttactactgtgcgaaagcccgttattacgatttttggggggggaatttcgatctctggggccgtggcacccaggtcactgtctcctca

SEQ ID NO: 602 - protein, 122 aa, Artificial Sequence
  Synthetic
EVQLLESGGGLVQPGGSLRLSCAASGFTFSSYAMNWVRQAPGKGLDWVSGISGNGGSTYYADSVKGRFTISRDISKNTLYVQMHSLRVEDTAVYYCAKARYYDFWGGNFDLWGRGTQVTVSS

SEQ ID NO: 603 - DNA, 24 bp, Artificial Sequence
  Synthetic
ggattcacgtttagtagctatgcc

SEQ ID NO: 604 - protein, 8 aa, Artificial Sequence
  Synthetic
GFTFSSYA

SEQ ID NO: 605 - DNA, 24 bp, Artificial Sequence
  Synthetic
atcagtggtaatggtggtagcacc

SEQ ID NO: 606 - protein, 8 aa, Artificial Sequence
  Synthetic
ISGNGGST

SEQ ID NO: 607 - DNA, 45 bp, Artificial Sequence
  Synthetic
gcgaaagcccgttattacgatttttggggggggaatttcgatctc

SEQ ID NO: 608 - protein, 15 aa, Artificial Sequence
  Synthetic
AKARYYDFWGGNFDL

SEQ ID NO: 609 - DNA, 324 bp, Artificial Sequence
  Synthetic
gaaattgtgttgacgcagtctccaggcaccctgtctttgtctccaggggaaagagccaccctctcctgcagggccagtcagagtgttagcatcaggtacttagcctggtatcagcagaaacctggccaggctcccaggctcctcatctatggtgcatccagcagggccactggcatcccagacaggttcagtgtcagtgtgtctgggacagacttcactctcaccatcactagactggagcctgaagattttgcagtctattactgtcagcaatatggtagttcaccgctcactttcggcggagggaccaaggtggagatcaaa

SEQ ID NO: 610 - protein, 108 aa, Artificial Sequence
  Synthetic
EIVLTQSPGTLSLSPGERATLSCRASQSVSIRYLAWYQQKPGQAPRLLIYGASSRATGIPDRFSVSVSGTDFTLTITRLEPEDFAVYYCQQYGSSPLTFGGGTKVEIK

SEQ ID NO: 611 - DNA, 21 bp, Artificial Sequence
  Synthetic
cagagtgttagcatcaggtac

SEQ ID NO: 612 - protein, 7 aa, Artificial Sequence
  Synthetic
QSVSIRY

SEQ ID NO: 613 - DNA, 9 bp, Artificial Sequence
  Synthetic
ggtgcatcc

SEQ ID NO: 614 - protein, 3 aa, Artificial Sequence
  Synthetic
GAS

SEQ ID NO: 615 - DNA, 27 bp, Artificial Sequence
  Synthetic
cagcaatatggtagttcaccgctcact

SEQ ID NO: 616 - protein, 9 aa, Artificial Sequence
  Synthetic
QQYGSSPLT

SEQ ID NO: 617 - DNA, 366 bp, Artificial Sequence
  Synthetic
gaggtgcagttgttggagtctgggggaggcttggtacagcctggggggtccctgagactctcctgtgcagcctctggattcacgtttagtagctatgccatgaactgggtccgccaggctccagggaaggggctggattgggtctcaggtatcagtggtaatggtggtagcacctactacgcagactccgtgaagggccggttcaccatctccagagacatttccaagaacacgctgtatgtgcaaatgcacagcctgagagtcgaggacacggccgtttactactgtgcgaaagcccgttattacgatttttggggggggaatttcgatctctggggccgtggcaccctggtcactgtctcctca

SEQ ID NO: 618 - protein, 122 aa, Artificial Sequence
  Synthetic
EVQLLESGGGLVQPGGSLRLSCAASGFTFSSYAMNWVRQAPGKGLDWVSGISGNGGSTYYADSVKGRFTISRDISKNTLYVQMHSLRVEDTAVYYCAKARYYDFWGGNFDLWGRGTLVTVSS

SEQ ID NO: 619 - DNA, 324 bp, Artificial Sequence; identical to SEQ ID NO: 609
  Synthetic

SEQ ID NO: 620 - protein, 108 aa, Artificial Sequence; identical to SEQ ID NO: 610
  Synthetic

SEQ ID NO: 621 - DNA, 366 bp, Artificial Sequence
  Synthetic
gaggtgcagctgttggagtctgggggaggcttggtacagcctggggggtccctgagactctcctgtgcagcctctggattcacgtttagtagctatgccatgagctgggtccgccaggctccagggaaggggctggagtgggtctcagctatcagtggtaatggtggtagcacctactacgcagactccgtgaagggccggttcaccatctccagagacaattccaagaacacgctgtatctgcaaatgaacagcctgagagccgaggacacggccgtatattactgtgcgaaagcccgttattacgatttttggggggggaatttcgatctctggggccgtggcaccctggtcactgtctcctca

SEQ ID NO: 622 - protein, 122 aa, Artificial Sequence
  Synthetic
EVQLLESGGGLVQPGGSLRLSCAASGFTFSSYAMSWVRQAPGKGLEWVSAISGNGGSTYYADSVKGRFTISRDNSKNTLYLQMNSLRAEDTAVYYCAKARYYDFWGGNFDLWGRGTLVTVSS

SEQ ID NO: 623 - DNA, 324 bp, Artificial Sequence
  Synthetic
gaaattgtgttgacgcagtctccaggcaccctgtctttgtctccaggggaaagagccaccctctcctgcagggccagtcagagtgttagcatcaggtacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcaatatggtagttcaccgctcactttcggcggagggaccaaggtggagatcaaa

SEQ ID NO: 624 - protein, 108 aa, Artificial Sequence
  Synthetic
EIVLTQSPGTLSLSPGERATLSCRASQSVSIRYLAWYQQKPGQAPRLLIYGASSRATGIPDRFSGSGSGTDFTLTISRLEPEDFAVYYCQQYGSSPLTFGGGTKVEIK

SEQ ID NO: 625 - DNA, 381 bp, Artificial Sequence
  Synthetic
caggttcagctggtgcagtctggacctgaggtgaagaaccctggggcctcagtgaaggtctcctgcaaggcttctggttacacctttaccacctatggtatcagttgggtacgacaggcccctggacaagggcttgagtggatgggatggatcagcggttacaatggtaaaacaaacgatgcacagaagttccaggacagagtcgccatgaccacagacacatccacgagcacagcctacatggagctgaggagcctgagatctgacgacacggccatttattactgttcgagagatcgtttagtagtaccacctgccctttattattcctactacgttatggacgtctggggccaagggaccacggtcaccgtctcctca

SEQ ID NO: 626 - protein, 127 aa, Artificial Sequence
  Synthetic
QVQLVQSGPEVKNPGASVKVSCKASGYTFTTYGISWVRQAPGQGLEWMGWISGYNGKTNDAQKFQDRVAMTTDTSTSTAYMELRSLRSDDTAIYYCSRDRLVVPPALYYSYYVMDVWGQGTTVTVSS

SEQ ID NO: 627 - DNA, 24 bp, Artificial Sequence
  Synthetic
ggttacacctttaccacctatggt

SEQ ID NO: 628 - protein, 8 aa, Artificial Sequence
  Synthetic
GYTFTTYG

SEQ ID NO: 629 - DNA, 24 bp, Artificial Sequence
  Synthetic
atcagcggttacaatggtaaaaca

SEQ ID NO: 630 - protein, 8 aa, Artificial Sequence
  Synthetic
ISGYNGKT

SEQ ID NO: 631 - DNA, 60 bp, Artificial Sequence
  Synthetic
tcgagagatcgtttagtagtaccacctgccctttattattcctactacgttatggacgtc

SEQ ID NO: 632 - protein, 20 aa, Artificial Sequence
  Synthetic
SRDRLVVPPALYYSYYVMDV

SEQ ID NO: 633 - DNA, 336 bp, Artificial Sequence
  Synthetic
gatgttgtgatgactcagtctccactctccctgcccgtcacccttggacagccggcctccatctcctgcaggtctagtcaaagcctcgtatacagtgatggaaacacctacttgaattggtttcagcagaggccaggtcaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggccgtacacttttggccaggggaccaagctggagatcaaa

SEQ ID NO: 634 - protein, 112 aa, Artificial Sequence; identical to SEQ ID NO: 336
  Synthetic

SEQ ID NO: 635 - DNA, 33 bp, Artificial Sequence; identical to SEQ ID NO: 323
  Synthetic

SEQ ID NO: 636 - protein, 11 aa, Artificial Sequence
  Synthetic
QSLVYSDGNTY

SEQ ID NO: 637 - DNA, 9 bp, Artificial Sequence
  Synthetic
aaggtttct

SEQ ID NO: 638 - protein, 3 aa, Artificial Sequence
  Synthetic
KVS

SEQ ID NO: 639 - DNA, 27 bp, Artificial Sequence
  Synthetic
atgcaaggtacacactggccgtacact

SEQ ID NO: 640 - protein, 9 aa, Artificial Sequence
  Synthetic
MQGTHWPYT

SEQ ID NO: 641 - DNA, 381 bp, Artificial Sequence; identical to SEQ ID NO: 625
  Synthetic

SEQ ID NO: 642 - protein, 127 aa, Artificial Sequence; identical to SEQ ID NO: 626
  Synthetic

SEQ ID NO: 643 - DNA, 336 bp, Artificial Sequence; identical to SEQ ID NO: 633
  Synthetic

SEQ ID NO: 644 - protein, 112 aa, Artificial Sequence; identical to SEQ ID NO: 336
  Synthetic

SEQ ID NO: 645 - DNA, 381 bp, Artificial Sequence
  Synthetic
caggttcagctggtgcagtctggagctgaggtgaagaagcctggggcctcagtgaaggtctcctgcaaggcttctggttacacctttaccacctatggtatcagctgggtgcgacaggcccctggacaagggcttgagtggatgggatggatcagcggttacaatggtaaaacaaactatgcacagaagctccagggcagagtcaccatgaccacagacacatccacgagcacagcctacatggagctgaggagcctgagatctgacgacacggccgtgtattactgttcgagagatcgtttagtagtaccacctgccctttattattcctactacgttatggacgtctgggggcaagggaccacggtcaccgtctcctca

SEQ ID NO: 646 - protein, 127 aa, Artificial Sequence
  Synthetic
QVQLVQSGAEVKKPGASVKVSCKASGYTFTTYGISWVRQAPGQGLEWMGWISGYNGKTNYAQKLQGRVTMTTDTSTSTAYMELRSLRSDDTAVYYCSRDRLVVPPALYYSYYVMDVWGQGTTVTVSS

SEQ ID NO: 647 - DNA, 336 bp, Artificial Sequence; identical to SEQ ID NO: 335
  Synthetic

SEQ ID NO: 648 - protein, 112 aa, Artificial Sequence; identical to SEQ ID NO: 336
  Synthetic

SEQ ID NO: 649 - DNA, 381 bp, Artificial Sequence
  Synthetic
caggttcagctggtgcagtctggacctgaggtgaagaaccctggggcctcagtgaaggtctcctgcaaggcttctggttacacctttaccacctatggtatcagttgggtacgacaggcccctggacaagggcttgagtggatgggatggatcagcggttacaatggtaaaacaaacgatgcacagaagttccaggacagagtcgccatgaccacagacacatccacgagcacagcctacatggagctgaggagcctgagatctgacgacacggccatttattactgttcgagagatcgtttagtagtaccacctgcccttaattattactactacgttatggacgtctggggccaagggaccacggtcaccgtctcctca

SEQ ID NO: 650 - protein, 127 aa, Artificial Sequence
  Synthetic
QVQLVQSGPEVKNPGASVKVSCKASGYTFTTYGISWVRQAPGQGLEWMGWISGYNGKTNDAQKFQDRVAMTTDTSTSTAYMELRSLRSDDTAIYYCSRDRLVVPPALNYYYYVMDVWGQGTTVTVSS

SEQ ID NO: 651 - DNA, 24 bp, Artificial Sequence
  Synthetic
ggttacacctttaccacctatggt

SEQ ID NO: 652 - protein, 8 aa, Artificial Sequence
  Synthetic
GYTFTTYG

SEQ ID NO: 653 - DNA, 24 bp, Artificial Sequence
  Synthetic
atcagcggttacaatggtaaaaca

SEQ ID NO: 654 - protein, 8 aa, Artificial Sequence
  Synthetic
ISGYNGKT

SEQ ID NO: 655 - DNA, 60 bp, Artificial Sequence
  Synthetic
tcgagagatcgtttagtagtaccacctgcccttaattattactactacgttatggacgtc

SEQ ID NO: 656 - protein, 20 aa, Artificial Sequence
  Synthetic
SRDRLVVPPALNYYYYVMDV

SEQ ID NO: 657 - DNA, 336 bp, Artificial Sequence; identical to SEQ ID NO: 633
  Synthetic

SEQ ID NO: 658 - protein, 112 aa, Artificial Sequence; identical to SEQ ID NO: 336
  Synthetic

SEQ ID NO: 659 - DNA, 33 bp, Artificial Sequence; identical to SEQ ID NO: 323
  Synthetic

SEQ ID NO: 660 - protein, 11 aa, Artificial Sequence
  Synthetic
QSLVYSDGNTY

SEQ ID NO: 661 - DNA, 9 bp, Artificial Sequence
  Synthetic
aaggtttct

SEQ ID NO: 662 - protein, 3 aa, Artificial Sequence
  Synthetic
KVS

SEQ ID NO: 663 - DNA, 27 bp, Artificial Sequence
  Synthetic
atgcaaggtacacactggccgtacact

SEQ ID NO: 664 - protein, 9 aa, Artificial Sequence
  Synthetic
MQGTHWPYT

SEQ ID NO: 665 - DNA, 381 bp, Artificial Sequence; identical to SEQ ID NO: 649
  Synthetic

SEQ ID NO: 666 - protein, 127 aa, Artificial Sequence; identical to SEQ ID NO: 650
  Synthetic

SEQ ID NO: 667 - DNA, 336 bp, Artificial Sequence; identical to SEQ ID NO: 633
  Synthetic

SEQ ID NO: 668 - protein, 112 aa, Artificial Sequence; identical to SEQ ID NO: 336
  Synthetic

SEQ ID NO: 669 - DNA, 381 bp, Artificial Sequence
  Synthetic
caggttcagctggtgcagtctggagctgaggtgaagaagcctggggcctcagtgaaggtctcctgcaaggcttctggttacacctttaccacctatggtatcagctgggtgcgacaggcccctggacaagggcttgagtggatgggatggatcagcggttacaatggtaaaacaaactatgcacagaagctccagggcagagtcaccatgaccacagacacatccacgagcacagcctacatggagctgaggagcctgagatctgacgacacggccgtgtattactgttcgagagatcgtttagtagtaccacctgcccttaattattactactacgttatggacgtctgggggcaagggaccacggtcaccgtctcctca

SEQ ID NO: 670 - protein, 127 aa, Artificial Sequence
  Synthetic
QVQLVQSGAEVKKPGASVKVSCKASGYTFTTYGISWVRQAPGQGLEWMGWISGYNGKTNYAQKLQGRVTMTTDTSTSTAYMELRSLRSDDTAVYYCSRDRLVVPPALNYYYYVMDVWGQGTTVTVSS

SEQ ID NO: 671 - DNA, 336 bp, Artificial Sequence; identical to SEQ ID NO: 335
  Synthetic

SEQ ID NO: 672 - protein, 112 aa, Artificial Sequence; identical to SEQ ID NO: 336
  Synthetic

SEQ ID NO: 673 - DNA, 381 bp, Artificial Sequence
  Synthetic
caggttcagctggtgcagtctggacctgaggtgaagaaccctggggcctcagtgaaggtctcctgcaaggcttctggttacacctttaccacctatggtatcagttgggtacgacaggcccctggacaagggcttgagtggatgggatggatcagcggttacaatggtaaaacaaacgatgcacagaagttccaggacagagtcgccatgaccacagacacatccacgagcacagcctacatggagctgaggagcctgagatctgacgacacggccatttattactgttcgagagatcgtttagtagtaccacctgccctttattattactactacgttatggacgtctggggccaagggaccacggtcaccgtctcctca

SEQ ID NO: 674 - protein, 127 aa, Artificial Sequence
  Synthetic
QVQLVQSGPEVKNPGASVKVSCKASGYTFTTYGISWVRQAPGQGLEWMGWISGYNGKTNDAQKFQDRVAMTTDTSTSTAYMELRSLRSDDTAIYYCSRDRLVVPPALYYYYYVMDVWGQGTTVTVSS

SEQ ID NO: 675 - DNA, 24 bp, Artificial Sequence
  Synthetic
ggttacacctttaccacctatggt

SEQ ID NO: 676 - protein, 8 aa, Artificial Sequence
  Synthetic
GYTFTTYG

SEQ ID NO: 677 - DNA, 24 bp, Artificial Sequence
  Synthetic
atcagcggttacaatggtaaaaca

SEQ ID NO: 678 - protein, 8 aa, Artificial Sequence
  Synthetic
ISGYNGKT

SEQ ID NO: 679 - DNA, 60 bp, Artificial Sequence
  Synthetic
tcgagagatcgtttagtagtaccacctgccctttattattactactacgttatggacgtc

SEQ ID NO: 680 - protein, 20 aa, Artificial Sequence
  Synthetic
SRDRLVVPPALYYYYYVMDV

SEQ ID NO: 681 - DNA, 336 bp, Artificial Sequence; identical to SEQ ID NO: 633
  Synthetic

SEQ ID NO: 682 - protein, 112 aa, Artificial Sequence; identical to SEQ ID NO: 336
  Synthetic

SEQ ID NO: 683 - DNA, 33 bp, Artificial Sequence; identical to SEQ ID NO: 323
  Synthetic

SEQ ID NO: 684 - protein, 11 aa, Artificial Sequence
  Synthetic
QSLVYSDGNTY

SEQ ID NO: 685 - DNA, 9 bp, Artificial Sequence
  Synthetic
aaggtttct

SEQ ID NO: 686 - protein, 3 aa, Artificial Sequence
  Synthetic
KVS

SEQ ID NO: 687 - DNA, 27 bp, Artificial Sequence
  Synthetic
atgcaaggtacacactggccgtacact

SEQ ID NO: 688 - protein, 9 aa, Artificial Sequence
  Synthetic
MQGTHWPYT

SEQ ID NO: 689 - DNA, 381 bp, Artificial Sequence; identical to SEQ ID NO: 673
  Synthetic

SEQ ID NO: 690 - protein, 127 aa, Artificial Sequence; identical to SEQ ID NO: 674
  Synthetic

SEQ ID NO: 691 - DNA, 336 bp, Artificial Sequence; identical to SEQ ID NO: 633
  Synthetic

SEQ ID NO: 692 - protein, 112 aa, Artificial Sequence; identical to SEQ ID NO: 336
  Synthetic

SEQ ID NO: 693 - DNA, 381 bp, Artificial Sequence
  Synthetic
caggttcagctggtgcagtctggagctgaggtgaagaagcctggggcctcagtgaaggtctcctgcaaggcttctggttacacctttaccacctatggtatcagctgggtgcgacaggcccctggacaagggcttgagtggatgggatggatcagcggttacaatggtaaaacaaactatgcacagaagctccagggcagagtcaccatgaccacagacacatccacgagcacagcctacatggagctgaggagcctgagatctgacgacacggccgtgtattactgttcgagagatcgtttagtagtaccacctgccctttattattactactacgttatggacgtctgggggcaagggaccacggtcaccgtctcctca

SEQ ID NO: 694 - protein, 127 aa, Artificial Sequence
  Synthetic
QVQLVQSGAEVKKPGASVKVSCKASGYTFTTYGISWVRQAPGQGLEWMGWISGYNGKTNYAQKLQGRVTMTTDTSTSTAYMELRSLRSDDTAVYYCSRDRLVVPPALYYYYYVMDVWGQGTTVTVSS

SEQ ID NO: 695 - DNA, 336 bp, Artificial Sequence; identical to SEQ ID NO: 335
  Synthetic

SEQ ID NO: 696 - protein, 112 aa, Artificial Sequence; identical to SEQ ID NO: 336
  Synthetic

SEQ ID NO: 697 - DNA, 384 bp, Artificial Sequence; identical to SEQ ID NO: 361
  Synthetic

SEQ ID NO: 698 - protein, 128 aa, Artificial Sequence; identical to SEQ ID NO: 362
  Synthetic

SEQ ID NO: 699 - DNA, 24 bp, Artificial Sequence
  Synthetic
ggattcaccttcagtgaccactac

SEQ ID NO: 700 - protein, 8 aa, Artificial Sequence
  Synthetic
GFTFSDHY

SEQ ID NO: 701 - DNA, 24 bp, Artificial Sequence
  Synthetic
attagtaatgatggtggtaccaaa

SEQ ID NO: 702 - protein, 8 aa, Artificial Sequence
  Synthetic
ISNDGGTK

SEQ ID NO: 703 - DNA, 63 bp, Artificial Sequence; identical to SEQ ID NO: 367
  Synthetic

SEQ ID NO: 704 - protein, 21 aa, Artificial Sequence
  Synthetic
ARDQGYIGYDSYYYYSYGMDV

SEQ ID NO: 705 - DNA, 321 bp, Artificial Sequence
  Synthetic
aaaattgtgttgacgcagtctccaggcaccctgcctttgtttccaggggaaagagccaccctctcctgtagggccagtcagagtgttaacaacaaattcttagcctggtaccagcagaaatctggccaggctcccaggctcctcatctatggtgcatccagcagggccactggcatcccagacaggttcagtggcagtgggtctgggaccgacttcactctcaccatcagcggactggagcctgaagattttgaagtgtattattgtcaagtatatggtaactcactcactttcggcggagggaccaaggtggagatcaag

SEQ ID NO: 706 - protein, 107 aa, Artificial Sequence
  Synthetic
KIVLTQSPGTLPLFPGERATLSCRASQSVNNKFLAWYQQKSGQAPRLLIYGASSRATGIPDRFSGSGSGTDFTLTISGLEPEDFEVYYCQVYGNSLTFGGGTKVEIK

SEQ ID NO: 707 - DNA, 21 bp, Artificial Sequence
  Synthetic
cagagtgttaacaacaaattc

SEQ ID NO: 708 - protein, 7 aa, Artificial Sequence
  Synthetic
QSVNNKF

SEQ ID NO: 709 - DNA, 9 bp, Artificial Sequence
  Synthetic
ggtgcatcc

SEQ ID NO: 710 - protein, 3 aa, Artificial Sequence
  Synthetic
GAS

SEQ ID NO: 711 - DNA, 24 bp, Artificial Sequence
  Synthetic
caagtatatggtaactcactcact

SEQ ID NO: 712 - protein, 8 aa, Artificial Sequence
  Synthetic
QVYGNSLT

SEQ ID NO: 713 - DNA, 384 bp, Artificial Sequence; identical to SEQ ID NO: 377
  Synthetic

SEQ ID NO: 714 - protein, 128 aa, Artificial Sequence; identical to SEQ ID NO: 378
  Synthetic

SEQ ID NO: 715 - DNA, 321 bp, Artificial Sequence
  Synthetic
gaaattgtgttgacgcagtctccaggcaccctgcctttgtttccaggggaaagagccaccctctcctgtagggccagtcagagtgttaacaacaaattcttagcctggtaccagcagaaatctggccaggctcccaggctcctcatctatggtgcatccagcagggccactggcatcccagacaggttcagtggcagtgggtctgggaccgacttcactctcaccatcagcggactggagcctgaagattttgaagtgtattattgtcaagtatatggtaactcactcactttcggcggagggaccaaggtggagatcaaa

SEQ ID NO: 716 - protein, 107 aa, Artificial Sequence
  Synthetic
EIVLTQSPGTLPLFPGERATLSCRASQSVNNKFLAWYQQKSGQAPRLLIYGASSRATGIPDRFSGSGSGTDFTLTISGLEPEDFEVYYCQVYGNSLTFGGGTKVEIK

SEQ ID NO: 717 - DNA, 384 bp, Artificial Sequence; identical to SEQ ID NO: 381
  Synthetic

SEQ ID NO: 718 - protein, 128 aa, Artificial Sequence; identical to SEQ ID NO: 382
  Synthetic

SEQ ID NO: 719 - DNA, 321 bp, Artificial Sequence; identical to SEQ ID NO: 383
  Synthetic

SEQ ID NO: 720 - protein, 107 aa, Artificial Sequence; identical to SEQ ID NO: 384
  Synthetic

SEQ ID NO: 721 - DNA, 378 bp, Artificial Sequence; identical to SEQ ID NO: 505
  Synthetic

SEQ ID NO: 722 - protein, 126 aa, Artificial Sequence; identical to SEQ ID NO: 506
  Synthetic

SEQ ID NO: 723 - DNA, 24 bp, Artificial Sequence
  Synthetic
ggttacacctttaccaactacgct

SEQ ID NO: 724 - protein, 8 aa, Artificial Sequence
  Synthetic
GYTFTNYA

SEQ ID NO: 725 - DNA, 24 bp, Artificial Sequence
  Synthetic
gtcagcgcttacaatggtcacaca

SEQ ID NO: 726 - protein, 8 aa, Artificial Sequence
  Synthetic
VSAYNGHT

SEQ ID NO: 727 - DNA, 57 bp, Artificial Sequence; identical to SEQ ID NO: 511
  Synthetic

SEQ ID NO: 728 - protein, 19 aa, Artificial Sequence
  Synthetic
ARGGVVVPVAPHFYNGMDV

SEQ ID NO: 729 - DNA, 336 bp, Artificial Sequence
  Synthetic
gatattgtgatgactcagtttccactctccctgcccgtcacccctggagagccggcctccatctcctgcaggtctagtcagagcctcctgcatattaatgaatacaactatttggattggtacctaaagaagccagggcagtctccacagctcctgatctatttgggttttaatcgggcctccggggtccctgacaggttcagtggcagtggatcaggcacagattttacactgaaaatcagcagagtggaggctgaggatgttggggtctattactgcatgcaagctcttcaaactccgtggacgttcggccaagggaccaaggtggaaatcaaa

SEQ ID NO: 730 - protein, 112 aa, Artificial Sequence
  Synthetic
DIVMTQFPLSLPVTPGEPASISCRSSQSLLHINEYNYLDWYLKKPGQSPQLLIYLGFNRASGVPDRFSGSGSGTDFTLKISRVEAEDVGVYYCMQALQTPWTFGQGTKVEIK

SEQ ID NO: 731 - DNA, 33 bp, Artificial Sequence; identical to SEQ ID NO: 515
  Synthetic

SEQ ID NO: 732 - protein, 11 aa, Artificial Sequence
  Synthetic
QSLLHINEYNY

SEQ ID NO: 733 - DNA, 9 bp, Artificial Sequence
  Synthetic
ttgggtttt

SEQ ID NO: 734 - protein, 3 aa, Artificial Sequence
  Synthetic
LGF

SEQ ID NO: 735 - DNA, 27 bp, Artificial Sequence
  Synthetic
atgcaagctcttcaaactccgtggacg

SEQ ID NO: 736 - protein, 9 aa, Artificial Sequence
  Synthetic
MQALQTPWT

SEQ ID NO: 737 - DNA, 378 bp, Artificial Sequence; identical to SEQ ID NO: 521
  Synthetic

SEQ ID NO: 738 - protein, 126 aa, Artificial Sequence; identical to SEQ ID NO: 522
  Synthetic

SEQ ID NO: 739 - DNA, 336 bp, Artificial Sequence
  Synthetic
gatattgtgatgactcagtctccactctccctgcccgtcacccctggagagccggcctccatctcctgcaggtctagtcagagcctcctgcatattaatgaatacaactatttggattggtacctaaagaagccagggcagtctccacagctcctgatctatttgggttttaatcgggcctccggggtccctgacaggttcagtggcagtggatcaggcacagattttacactgaaaatcagcagagtggaggctgaggatgttggggtctattactgcatgcaagctcttcaaactccgtggacgttcggccaagggaccaaggtggaaatcaaa

SEQ ID NO: 740 - protein, 112 aa, Artificial Sequence
  Synthetic
DIVMTQSPLSLPVTPGEPASISCRSSQSLLHINEYNYLDWYLKKPGQSPQLLIYLGFNRASGVPDRFSGSGSGTDFTLKISRVEAEDVGVYYCMQALQTPWTFGQGTKVEIK

SEQ ID NO: 741 - DNA, 378 bp, Artificial Sequence; identical to SEQ ID NO: 525
  Synthetic

SEQ ID NO: 742 - protein, 126 aa, Artificial Sequence; identical to SEQ ID NO: 526
  Synthetic

SEQ ID NO: 743 - DNA, 336 bp, Artificial Sequence; identical to SEQ ID NO: 527
  Synthetic

SEQ ID NO: 744 - protein, 112 aa, Artificial Sequence; identical to SEQ ID NO: 528
  Synthetic

SEQ ID NO: 745 - protein, 8 aa, Artificial Sequence
  Synthetic
  VARIANT (1)...(8): Xaa = Any amino acid
XXXXXXXX

SEQ ID NO: 746 - protein, 8 aa, Artificial Sequence
  Synthetic
  VARIANT (1)...(8): Xaa - Any amino acid
XXXXXXXX

SEQ ID NO: 747 - protein, 20 aa, Artificial Sequence
  Synthetic
  VARIANT (1)...(20): Xaa = Any amino acid
XXXXXXXXXXXXXXXXXXXX

SEQ ID NO: 748 - protein, 12 aa, Artificial Sequence
  Synthetic
  VARIANT (1)...(12): Xaa = Any amino acid
XXXXXXXXXXXX

SEQ ID NO: 749 - protein, 3 aa, Artificial Sequence
  Synthetic
  VARIANT (1)...(3): Xaa = Any amino acid
XXX

SEQ ID NO: 750 - protein, 9 aa, Artificial Sequence
  Synthetic
  VARIANT (1)...(9): Xaa = Any amino acid
XXXXXXXXX

SEQ ID NO: 751 - protein, 330 aa, Artificial Sequence [sequence omitted]

SEQ ID NO: 752 - protein, 327 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 753 - protein, 327 aa, Artificial Sequence
  Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 754 - DNA, 2076 bp, Homo sapiens
atgggcaccgtcagctccaggcggtcctggtggccgctgccactgctgctgctgctgctgctgctcctgggtcccgcgggcgcccgtgcgcaggaggacgaggacggcgactacgaggagctggtgctagccttgcgttccgaggaggacggcctggccgaagcacccgagcacggaaccacagccaccttccaccgctgcgccaaggatccgtggaggttgcctggcacctacgtggtggtgctgaaggaggagacccacctctcgcagtcagagcgcactgcccgccgcctgcaggcccaggctgcccgccggggatacctcaccaagatcctgcatgtcttccatggccttcttcctggcttcctggtgaagatgagtggcgacctgctggagctggccttgaagttgccccatgtcgactacatcgaggaggactcctctgtctttgcccagagcatcccgtggaacctggagcggattacccctccacggtaccgggcggatgaataccagccccccgacggaggcagcctggtggaggtgtatctcctagacaccagcatacagagtgaccaccgggaaatcgagggcagggtcatggtcaccgacttcgagaatgtgcccgaggaggacgggacccgcttccacagacaggccagcaagtgtgacagtcatggcacccacctggcaggggtggtcagcggccgggatgccggcgtggccaagggtgccagcatgcgcagcctgcgcgtgctcaactgccaagggaagggcacggttagcggcaccctcataggcctggagtttattcggaaaagccagctggtccagcctgtggggccactggtggtgctgctgcccctggcgggtgggtacagccgcgtcctcaacgccgcctgccagcgcctggcgagggctggggtcgtgctggtcaccgctgccggcaacttccgggacgatgcctgcctctactccccagcctcagctcccgaggtcatcacagttggggccaccaatgcccaggaccagccggtgaccctggggactttggggaccaactttggccgctgtgtggacctctttgccccaggggaggacatcattggtgcctccagcgactgcagcacctgctttgtgtcacagagtgggacatcacaggctgctgcccacgtggctggcattgcagccatgatgctgtctgccgagccggagctcaccctggccgagttgaggcagagactgatccacttctctgccaaagatgtcatcaatgaggcctggttccctgaggaccagcgggtactgacccccaacctggtggccgccctgccccccagcacccatggggcaggttggcagctgttttgcaggactgtgtggtcagcacactcggggcctacacggatggccacagccatcgcccgctgcgccccagatgaggagctgctgagctgctccagtttctccaggagtgggaagcggcggggcgagcgcatggaggcccaagggggcaagctggtctgccgggcccacaacgcttttgggggtgagggtgtctacgccattgccaggtgctgcctgctaccccaggccaactgcagcgtccacacagctccaccagctgaggccagcatggggacccgtgtccactgccaccaacagggccacgtcctcacaggctgcagctcccactgggaggtggaggaccttggcacccacaagccgcctgtgctgaggccacgaggtcagcccaaccagtgcgtgggccacagggaggccagcatccacgcttcctgctgccatgccccaggtctggaatgcaaagtcaaggagcatggaatcccggcccctcaggagcaggtgaccgtggcctgcgaggagggctggaccctgactggctgcagtgccctccctgggacctcccacgtcctgggggcctacgccgtagacaacacgtgtgtagtcaggagccgggacgtcagcactacaggcagcaccagcgaagaggccgtgacagccgttgccatctgctgccggagccggcacctggcgcaggcctcccaggagctccag

SEQ ID NO: 755 - protein, 692 aa, Homo sapiens
MGTVSSRRSWWPLPLLLLLLLLLGPAGARAQEDEDGDYEELVLALRSEEDGLAEAPEHGTTATFHRCAKDPWRLPGTYVVVLKEETHLSQSERTARRLQAQAARRGYLTKILHVFHGLLPGFLVKMSGDLLELALKLPHVDYIEEDSSVFAQSIPWNLERITPPRYRADEYQPPDGGSLVEVYLLDTSIQSDHREIEGRVMVTDFENVPEEDGTRFHRQASKCDSHGTHLAGVVSGRDAGVAKGASMRSLRVLNCQGKGTVSGTLIGLEFIRKSQLVQPVGPLVVLLPLAGGYSRVLNAACQRLARAGVVLVTAAGNFRDDACLYSPASAPEVITVGATNAQDQPVTLGTLGTNFGRCVDLFAPGEDIIGASSDCSTCFVSQSGTSQAAAHVAGIAAMMLSAEPELTLAELRQRLIHFSAKDVINEAWFPEDQRVLTPNLVAALPPSTHGAGWQLFCRTVWSAHSGPTRMATAIARCAPDEELLSCSSFSRSGKRRGERMEAQGGKLVCRAHNAFGGEGVYAIARCCLLPQANCSVHTAPPAEASMGTRVHCHQQGHVLTGCSSHWEVEDLGTHKPPVLRPRGQPNQCVGHREASIHASCCHAPGLECKVKEHGIPAPQEQVTVACEEGWTLTGCSALPGTSHVLGAYAVDNTCVVRSRDVSTTGSTSEEAVTAVAICCRSRHLAQASQELQ

SEQ ID NO: 756 - protein, 692 aa, Macaca mulata
MGTVSSRRSWWPLPLPLLLLLLLGPAGARAQEDEDGDYEELVLALRSEEDGLADAPEHGATATFHRCAKDPWRLPGTYVVVLKEETHRSQSERTARRLQAQAARRGYLTKILHVFHHLLPGFLVKMSGDLLELALKLPHVDYIEEDSSVFAQSIPWNLERITPARYRADEYQPPKGGSLVEVYLLDTSIQSDHREIEGRVMVTDFESVPEEDGTRFHRQASKCDSHGTHLAGVVSGRDAGVAKGAGLRSLRVLNCQGKGTVSGTLIGLEFIRKSQLVQPVGPLVVLLPLAGGYSRVFNAACQRLARAGVVLVTAAGNFRDDACLYSPASAPEVITVGATNAQDQPVTLGTLGTNFGRCVDLFAPGEDIIGASSDCSTCFVSRSGTSQAAAHVAGIAAMMLSAEPELTLAELRQRLIHFSAKDVINEAWFPEDQRVLTPNLVAALPPSTHRAGWQLFCRTVWSAHSGPTRMATAVARCAQDEELLSCSSFSRSGKRRGERIEAQGGKRVCRAHNAFGGEGVYAIARCCLLPQVNCSVHTAPPAGASMGTRVHCHQQGHVLTGCSSHWEVEDLGTHKPPVLRPRGQPNQCVGHREASIHASCCHAPGLECKVKEHGIPAPQEQVIVACEDGWTLTGCSPLPGTSHVLGAYAVDNTCVVRSRDVSTTGSTSKEAVAAVAICCRSRHLVQASQELQ

SEQ ID NO: 757 - protein, 694 aa, Mus muscular
MGTHCSAWLRWPLLPLLPPLLLLLLLLCPTGAGAQDEDGDYEELMLALPSQEDGLADEAAHVATATFRRCSKEAWRLPGTYIVVLMEETQRLQIEQTAHRLQTRAARRGYVIKVLHIFYDLFPGFLVKMSSDLLGLALKLPHVEYIEEDSFVFAQSIPWNLERIIPAWHQTEEDRSPDGSSQVEVYLLDTSIQGAHREIEGRVTITDFNSVPEEDGTRFHRQASKCDSHGTHLAGVVSGRDAGVAKGTSLHSLRVLNCQGKGTVSGTLIGLEFIRKSQLIQPSGPLVVLLPLAGGYSRILNAACRHLARTGVVLVAAAGNFRDDACLYSPASAPEVITVGATNAQDQPVTLGTLGTNFGRCVDLFAPGKDIIGASSDCSTCFMSQSGTSQAAAHVAGIVARMLSREPTLTLAELRQRLIHFSTKDVINMAWFPEDQQVLTPNLVATLPPSTHETGGQLLCRTVWSAHSGPTRTATATARCAPEEELLSCSSFSRSGRRRGDWIEAIGGQQVCKALNAFGGEGVYAVARCCLVPRANCSIHNTPAARAGLETHVHCHQKDHVLTGCSFHWEVEDLSVRRQPALRSRRQPGQCVGHQAASVYASCCHAPGLECKIKEHGISGPSEQVTVACEAGWTLTGCNVLPGASLTLGAYSVDNLCVARVHDTARADRTSGEATVAAAICCRSRPSAKASWVQ

SEQ ID NO: 758 - protein, 653 aa, Homo sapiens
EFRCHDGKCISRQFVCDSDRDCLDGSDEASCPVLTCGPASFQCNSSTCIPQLWACDNDPDCEDGSDEWPQRCRGLYVFQGDSSPCSAFEFHCLSGECIHSSWRCDGGPDCKDKSDEENCAVATCRPDEFQCSDGNCIHGSRQCDREYDCKDMSDEVGCVNVTLCEGPNKFKCHSGECITLDKVCNMARDCRDWSDEPIKECGTNECLDNNGGCSHVCNDLKIGYECLCPDGFQLVAQRRCEDIDECQDPDTCSQLCVNLEGGYKCQCEEGFQLDPHTKACKAVGSIAYLFFTNRHEVRKMTLDRSEYTSLIPNLRNVVALDTEVASNRIYWSDLSQRMICSTQLDRAHGVSSYDTVISRDIQAPDGLAVDWIHSNIYWTDSVLGTVSVADTKGVKRKTLFRENGSKPRAIVVDPVHGFMYWTDWGTPAKIKKGGLNGVDIYSLVTENIQWPNGITLDLLSGRLYWVDSKLHSISSIDVNGGNRKTILEDEKRLAHPFSLAVFEDKVFWTDIINEAIFSANRLTGSDVNLLAENLLSPEDMVLFHNLTQPRGVNWCERTTLSNGGCQYLCLPAPQINPHSPKFTCACPDGMLLARDMRSCLTEAEAAVATQETSTVRLKVSSTAVRTQHTTTRPVPDTSRLPGATPGLTTVEIV

SEQ ID NO: 759 - protein, 753 aa, Homo sapiens
MERRAWSLQCTAFVLFCAWCALNSAKAKRQFVNEWAAEIPGGPEAASAIAEELGYDLLGQIGSLENHYLFKHKNHPRRSRRSAFHITKRLSDDDRVIWAEQQYEKERSKRSALRDSALNLFNDPMWNQQWYLQDTRMTAALPKLDLHVIPVWQKGITGKGVVITVLDDGLEWNHTDIYANYDPEASYDFNDNDHDPFPRYDPTNENKHGTRCAGEIAMQANNHKCGVGVAYNSKVGGIRMLDGIVTDAIEASSIGFNPGHVDIYSASWGPNDDGKTVEGPGRLAQKAFEYGVKQGRQGKGSIFVWASGNGGRQGDNCDCDGYTDSIYTISISSASQQGLSPWYAEKCSSTLATSYSSGDYTDQRITSADLHNDCTETHTGTSASAPLAAGIFALALEANPNLTWRDMQHLVVWTSEYDPLANNPGWKKNGAGLMVNSRFGFGLLNAKALVDLADPRTWRSVPEKKECVVKDNDFEPRALKANGEVIIEIPTRACEGQENAIKSLEHVQFEATIEYSRRGDLHVTLTSAAGTSTVLLAERERDTSPNGFKNWDFMSVHTWGENPIGTWTLRITDMSGRIQNEGRIVNWKLILHGTSSQPEHMKQPRVYTSYNTVQNDRRGVEKMVDPGEEQPTQENPKENTLVSKSPSSSSVGGRRDELEEGAPSQAMLRLLQSAFSKNSPPKQSPKKSPSAKLNIPYENFYEALEKLNKPSQLKDSEDSLYNDYVDVFYNTKPYKHRDDRLLQALVDILNEEN

SEQ ID NO: 760 - protein, 785 aa, Homo sapiens
MPKGRQKVPHLDAPLGLPTCLWLELAGLFLLVPWVMGLAGTGGPDGQGTGGPSWAVHLESLEGDGEEETLEQQADALAQAAGLVNAGRIGELQGHYLFVQPAGHRPALEVEAIRQQVEAVLAGHEAVRWHSEQRLLRRAKRSVHFNDPKYPQQWHLNNRRSPGRDINVTGVWERNVTGRGVTVVVVDDGVEHTIQDIAPNYSPEGSYDLNSNDPDPMPHPDVENGNHHGTRCAGEIAAVPNNSFCAVGVAYGSRIAGIRVLDGPLTDSMEAVAFNKHYQINDIYSCSWGPDDDGKTVDGPHQLGKAALQHGVIAGRQGFGSIFVVASGNGGQHNDNCNYDGYANSIYTVTIGAVDEEGRMPFYAEECASMLAVTFSGGDKMLRSIVTTDWDLQKGTGCTEGHTGTSAAAPLAAGMIALMLQVRPCLTWRDVQHIIVFTATRYEDRRAEWVTNEAGFSHSHQHGFGLLNAWRLVNAAKIWTSVPYLASYVSPVLKENKAIPQSPRSLEVLWNVSRMDLEMSGLKTLEHVAVTVSITHPRRGSLELKLFCPSGMMSLIGAPRSMDSDPNGFNDWTFSTVRCWGERARGTYRLVIRDVGDESFQVGILRQWQLTLYGSVWSAVDIRDRQRLLESAMSGKYLHDDFALPCPPGLKIPEEDGYTITPNTLKTLVLVGCFTVFWTVYYMLEVYLSQRNVASNQVCRSGPCHWPHRSRKAKEEGTELESVPLCSSKDPDEVETESRGPPTTSDLLAPDLLEQGDWSLSQNKSALDCPHQHLDVPHGKEEQIC

SEQ ID NO: 761 - protein, 692 aa, Macaca fascicularis
MGTVSSRRSWWPLPLPLLLLLLLGPAGARAQEDEDGDYEELVLALRSEEDGLADAPEHGATATFHRCAKDPWRLPGTYVVVLKEETHRSQSERTARRLQAQAARRGYLTKILHVFHHLLPGFLVKMSGDLLELALKLPHVDYIEEDSSVFAQSIPWNLERITPARYRADEYQPPKGGSLVEVYLLDTSIQSDHREIEGRVMVTDFESVPEEDGTRFHRQASKCDSHGTHLAGVVSGRDAGVAKGAGLRSLRVLNCQGKGTVSGTLIGLEFIRKSQLVQPVGPLVVLLPLAGGYSRVFNAACQRLARAGVVLVTAAGNFRDDACLYSPASAPEVITVGATNAQDQPVTLGTLGTNFGRCVDLFAPGEDIIGASSDCSTCFVSRSGTSQAAAHVAGIAAMMLSAEPELTLAELRQRLIHFSAKDVINEAWFPEDQRVLTPNLVAALPPSTHRAGWQLFCRTVWSAHSGPTRMATAVARCAQDEELLSCSSFSRSGKRRGERIEAQGGKRVCRAHNAFGGEGVYAIARCCLLPQVNCSVHTAPPAGASMGTRVHCHQQGHVLTGCSSHWEVEDLGTHKPPVLRPRGQPNQCVGHREASIHASCCHAPGLECKVKEHGIPAPQEQVIVACEDGWTLTGCSALPGTSHVLGAYAVDNTCVVRSRDVSTTGSTSEEAVAAVAICCRSRHLVQASQELQ

SEQ ID NO: 762 - protein, 698 aa, Mesocricetus auratus
MGTSCSARPRWLLSPLLLLLLLLRYMGASAQDEDAEYEELMLTLQSQDDGLADETDEAPQGATAAFHRCPEEAWRVPGTYIVMLAEEAQWVHIEQTMHRLQTQAARRGYVIKIQHIFYDFLPAFVVKMSSDLLDLALKLPHVKYIEEDSLVFAQSIPWNLDRIIPAGRQAQEYSSSRKVPSGSGQVEVYLLDTSIQSDHREIEGRVTVTDFNSVPEEDGTRFHRQASKCDSHGTHLAGVVSGRDAGVAKGTILHGLRVLNCQGKGIVSGILTGLEFIWKSQLMQPSGPQVVLLPLAGRYSRVLNTACQHLARTGVVLVAAAGNFRDDACLYSPASAPEVITVGATDVQDQPVTLGTLGTNFGRCVDLFAPGKDIIGASSDCSACFMSQSGTSQAAAHVAGIVAMMLTLEPELTLTELRQRLIHFSTKDAINMAWFPEDQRVLTPNLVATLPPSTHGTGGQLLCRTVWSAHSGPTRAATATARCAPGEELLSCSSFSRSGRRRGDRIEAAGTQQVCKALNAFGGEGVYAVARCCLLPRANCSIHTTPAARTSLETHAHCHQKDHVLTGCSLHWEVEGIGVQPLAVLRSRHQPGQCTGHREASVHASCCHAPGLECKIKEHGISGPAEQVTVACEAGWTLTGCNVLPGAFITLGAYAVDNTCVARSRVTDTAGRTGEEATVAAAICCRNRPSAKASWVHQ

SEQ ID NO: 763 - protein, 691 aa, Rattus norvegicus
MGIRCSTWLRWPLSPQLLLLLLLCPTGSRAQDEDGDYEELMLALPSQEDSLVDEASHVATATFRRCSKEAWRLPGTYVVVLMEETQRLQVEQTAHRLQTWAARRGYVIKVLHVFYDLFPGFLVKMSSDLLGLALKLPHVEYIEEDSLVFAQSIPWNLERIIPAWQQTEEDSSPDGSSQVEVYLLDTSIQSGHREIEGRVTITDFNSVPEEDGTRFHRQASKCDSHGTHLAGVVSGRDAGVAKGTSLHSLRVLNCQGKGTVSGTLIGLEFIRKSQLIQPSGPLVVLLPLAGGYSRILNTACQRLARTGVVLVAAAGNFRDDACLYSPASAPEVITVGATNAQDQPVTLGTLGTNFGRCVDLFAPGKDIIGASSDCSTCYMSQSGTSQAAAHVAGIVAMMLNRDPALTLAELRQRLILFSTKDVINMAWFPEDQRVLTPNRVATLPPSTQETGGQLLCRTVWSAHSGPTRTATATARCAPEEELLSCSSFSRSGRRRGDRIEAIGGQQVCKALNAFGGEGVYAVARCCLLPRVNCSIHNTPAARAGPQTPVHCHQKDHVLTGCSFHWEVENLRAQQQPLLRSRHQPGQCVGHQEASVHASCCHAPGLECKIKEHGIAGPAEQVTVACEAGWTLTGCNVLPGASLPLGAYSVDNVCVARIRDAGRADRTSEEATVAAAICCRSRPSAKASWVHQ

SEQ ID NO: 764 - protein, 447 aa, Artificial Sequence
  synthetic
EVQLVESGGGLVQPGGSLRLSCAASGFTFNNYAMNWVRQAPGKGLDWVSTISGSGGTTNYADSVKGRFIISRDSSKHTLYLQMNSLRAEDTAVYYCAKDSNWGNFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 765 - protein, 220 aa, Artificial Sequence
  synthetic
DIVMTQSPDSLAVSLGERATINCKSSQSVLYRSNNRNFLGWYQQKPGQPPNLLIYWASTRESGVPDRFSGSGSGTDFTLTISSLQAEDVAVYYCQQYYTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2014/0356370 A1
(43) Pub. Date: Dec. 4, 2014

(54) METHODS FOR TREATING AUTOSOMAL DOMINANT HYPERCHOLESTEROLEMIA ASSOCIATED WITH PCSK9 GAIN-OF-FUNCTION MUTATIONS

(71) Applicant: REGENERON PHARMACEUTICALS, INC., Tarrytown, NY (US)

(72) Inventors: Gary SWERGOLD, New Rochelle, NY (US); Scott MELLIS, New Rochelle, NY (US); William J. SASIELA, Bloomingdale, NJ (US)

(73) Assignee: REGENERON PHARMACEUTICALS, INC., Tarrytown, NY (US)

(21) Appl. No.: 14/290,462

(22) Filed: May 29, 2014

Related U.S. Application Data
(60) Provisional application No. 61/828,730, filed on May 30, 2013.
(60) Provisional application No. 61/889,553, filed on Oct. 11, 2013.
(60) Provisional application No. 61/901,212, filed on Nov. 7, 2013.

(51) Int. Cl. C07K 16/40; C12Q 1/68

(52) U.S. Cl. CPC C07K 16/40; C12Q 1/68; A61K 2039/505
USPC 424/142.1; 435/6.1

(57) ABSTRACT

The present invention provides methods for treating autosomal dominant hypercholesterolemia (ADH). According to certain embodiments, the ADH is caused by or associated with a gain-of-function mutation (GOFm) in a gene encoding PCSK9. The present invention therefore includes methods comprising selecting a patient who carries a GOFm in one or both alleles of the PCSK9 gene, and administering to the patient a pharmaceutical composition comprising a PCSK9 inhibitor. In certain embodiments, the PCSK9 inhibitor is an anti-PCSK9 antibody such as the exemplary antibody referred to herein as mAb316P.

### CROSS-REFERENCE TO RELATED APPLICATIONS

This application claims the benefit under 35 U.S.C. §119(e) of U.S. provisional application Nos. 61/828,730, filed on May 30, 2013; 61/889,553, filed on Oct. 11, 2013; and 61/901,212, filed on Nov. 7, 2013, the disclosures of which are herein incorporated by reference in their entireties.

### FIELD OF THE INVENTION

The present invention relates to the field of therapeutic treatments of diseases and disorders which are associated with elevated levels of lipids and lipoproteins. More specifically, the invention relates to the administration of PCSK9 inhibitors to treat autosomal dominant hypercholesterolemia in patients with one or more gain of function mutations in the PCSK9 gene.

### BACKGROUND

Autosomal dominant hypercholesterolemia (ADH) is characterized by inherited high serum LDL cholesterol (LDL-C) levels and early onset cardiovascular disease. Causes of ADH include mutations in the LDL receptor (LDLR) and its ligand apolipoprotein B (apoB), and (in ˜1% of patients) gain-of-function mutations (GOFm) in proprotein convertase subtilisin/kexin type 9 (PCSK9), a potent modulator of hepatocyte LDLR.

PCSK9 is a proprotein convertase belonging to the proteinase K subfamily of the secretory subtilase family. The use of PCSK9 inhibitors (anti-PCSK9 antibodies) to reduce serum total cholesterol, LDL cholesterol and serum triglycerides is described in U.S. Pat. Nos. 8,062,640, 8,357,371, and US Patent Application Publication No. 2013/0064834.

### BRIEF SUMMARY OF THE INVENTION

The present invention provides methods for treating autosomal dominant hypercholesterolemia (ADH), e.g., ADH caused by or associated with PCSK9 gain-of-function mutations. The methods of the present invention comprise selecting a patient with ADH who carries a gain-of-function mutation (GOFm) in one or both alleles of the PCSK9 gene, and administering to the patient a pharmaceutical composition comprising a PCSK9 inhibitor.

PCSK9 inhibitors which may be administered in accordance with the methods of the present invention include, e.g., anti-PCSK9 antibodies or antigen-binding fragments thereof. Specific exemplary anti-PCSK9 antibodies which may be used in the practice of the methods of the present invention include any antibodies or antigen-binding fragments as set forth in U.S. Pat. No. 8,357,371, and/or disclosed herein.

The PCSK9 inhibitor may be administered to a subject subcutaneously or intravenously. Furthermore, the PCSK9 inhibitor may be administered to a patient who is on a therapeutic statin regimen at the time of therapeutic intervention.

Other embodiments of the present invention will become apparent from a review of the ensuing detailed description.

### BRIEF DESCRIPTION OF THE FIGURES

FIG. 1A shows the number of individuals (n) with various PCSK9 mutation from the different countries analyzed and the number of pedigrees (P) from each corresponding country. “Min” indicates the minimum number of pedigrees (some individuals did not have pedigree indicated).

FIG. 1B provides a summary of clinical data of patients with a familial gain-of-function mutation (GOFm) in PCSK9.

FIG. 2 shows the distribution of baseline LDL-C for patients with PCSK9 GOFm without LDLR mutations. P-value refers to whether the LDL-C mean levels for a particular mutation are significantly higher (up arrow) or lower (down arrow) than the population of genetic variants.

FIG. 3 shows the mean total cholesterol, LDL-C, HDL-C and triglyceride levels for subjects with PCSK9 GOFm (no LDLR mutation, N=134); FH (LDLR mutation, N=2126 [subjects matched for age and gender from the Dutch registry]); and FDB (ApoB mutation, N=470 [subjects matched for age and gender from the Dutch registry]). (†) indicates P<0.0001 vs. PCSK9-GOFm; (‡) indicates P<0.001 vs. PCSK9-GOFm.

FIG. 4 shows PCSK9-GOFm response to current lipid lowering therapy (LLT) in terms of reported lipid profiles before and after medication. (*) indicates P<0.0001; (†) indicates P<0.0002. LLTs were statins (atorvastatin 10-80 mg, cerivastatin 0.3 mg, fluvastatin 30 mg, pitavastatin 1-4 mg, pravastatin 10-20 mg, rosuvastatin 5-40 mg, simvastatin 20-80 mg), ezetimibe 10 mg, or fenofibrate 100-250 mg.

FIG. 5 shows PCSK9-GOFm response to current lipid lowering therapies (LLTs) in terms of achievement of LDL-C goals for patients with PCSK9-GOFm, no LDLR mutation, baseline and on-treatment LDL-C (N=63). LDL-C goals are based on NCEP-ATP III guidelines (Grundy et al., Circulation 2004, 110:227-239).

FIG. 6 is a schematic of the clinical trial described in Example 3. Group A patients received placebo (PBO) on days −14, 57 85 and 99, and mAb316P (mAb) on days 1, 15, 29, 43 and 71. Group B patients received placebo (PBO) on days −14, 1, 71 and 99, and mAb316P (mAb) on days 15, 29, 43, 57 and 85. The single-blind placebo run-in phase for both groups was from day −14 to day 1; the double-blind treatment period for both groups was from day 1 to day 99. The follow-up phase was from day 99 to day 155.

FIG. 7 shows the percent change from baseline in LDL-C, ApoB, TG, HDL-C, ApoA1 and Lp(a) at Week 8 for all 13 patients in the clinical trial described in Example 3.

FIG. 8 shows the change in measured LDL-C (mg/dL) in Group A and Group B patients over time, following treatment as shown by the gray and white arrows along the x-axis.

FIG. 9 shows the change from baseline in free PCSK9(%) in Group A and Group B patients over time, following treatment as shown by the gray and white arrows along the x-axis.

FIG. 10 shows the change from baseline in LDL-C (%) in patients with various PCSK9 GOF mutations (D364Y, L108R, S127R and R218S) over time, following treatment as shown by the gray and white arrows along the x-axis.

FIG. 11 shows the change from baseline in free PCSK9(%) in patients with various PCSK9 GOF mutations (D364Y, L108R, S127R and R218S) over time, following treatment as shown by the gray and white arrows along the x-axis.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice of the present invention, the preferred methods and materials are now described. All publications mentioned herein are incorporated herein by reference to describe in their entirety.

### Methods for Treating Autosomal Dominant Hypercholesterolemia

The present invention provides methods for treating autosomal dominant hypercholesterolemia (ADH). In certain embodiments, the present invention provides methods for treating ADH caused by, or associated with, one or more gain-of-function mutations (GOFm) in PCSK9. As used herein, the expression “GOFm in PCSK9” means any mutation in one or both alleles encoding the PCSK9 protein which result in an amino acid change that enhances PCSK9's ability to reduce levels of LDL receptor, or is otherwise associated with or correlated with hypercholesterolemia in human patients. Exemplary GOF mutations in PCSK9 include V4I, E32K, D35Y, E48K, P71L, R96C, L108R, S127R, D129N, R215H, F216L, R218S, R357H, D374H, D374Y, S465L and R496W.

The methods of the present invention comprise selecting a patient who carries a GOFm in one or both alleles of the PCSK9 gene, and administering to the patient a pharmaceutical composition comprising a PCSK9 inhibitor. The GOFm can be any PCSK9 GOF mutation. According to certain embodiments, the GOFm encodes a PCSK9 variant protein comprising an amino acid substitution selected from the group consisting of V4I, E32K, D35Y, E48K, P71L, R96C, L108R, S127R, D129N, R215H, F216L, R218S, R357H, D374H, D374Y, S465L and R496W. Methods for determining whether a patient carries a PCSK9 GOFm are known in the art. For example, DNA sequence analysis of the PCSK9 gene can be used in the context of the present invention to identify a patient carrying one or more copies of an allele that encodes a PCSK9 GOFm. Proteomics based approaches for identifying PCSK9 GOF mutations are also included within the scope of the methods of the present invention.

In certain embodiments, the patient may be further selected on the basis of exhibiting one or more additional characteristics or risk factors for hypercholesterolemia. For example, the present invention includes methods in which a patient who carries a PCSK9 GOFm is further selected for treatment on the basis of having a plasma LDL-C level of greater than or equal to about 70 mg/dL (i.e., prior to administration of the pharmaceutical composition comprising the PCSK9 inhibitor). In other embodiments, a patient who carries a PCSK9 GOFm is further selected on the basis of having a body mass index (BMI) of greater than about 18.0 kg/m2 prior to administration of the pharmaceutical composition comprising the PCSK9 inhibitor.

According to the present invention, the patient may be on a background lipid lowering therapy at the time of or prior to administration of the pharmaceutical composition comprising the PCSK9 inhibitor. Examples of background lipid lowering therapy include statins [e.g., cerivastatin, atorvastatin, simvastatin, pitavastatin, rosuvastatin, fluvastatin, lovastatin, pravastatin, etc.], ezetimibe, fibrates, niacin, omega-3 fatty acids, and bile acid resins.

The patient who is to be treated with the methods of the present invention may be selected on the basis of one or more factors selected from the group consisting of age (e.g., older than 40, 45, 50, 55, 60, 65, 70, 75, or 80 years), race, national origin, gender (male or female), exercise habits (e.g., regular exerciser, non-exerciser), other preexisting medical conditions (e.g., type-II diabetes, high blood pressure, etc.), and current medication status (e.g., currently taking statins [e.g., cerivastatin, atorvastatin, simvastatin, pitavastatin, rosuvastatin, fluvastatin, lovastatin, pravastatin, etc.], beta blockers, niacin, etc.). The methods of the present invention can also be used to treat ADH in patients who are intolerant of, non-responsive to, or inadequately responsive to conventional statin therapy. Potential patients can be selected/screened on the basis of one or more of the foregoing selection factors (e.g., by questionnaire, diagnostic evaluation, etc.) before being treated with the methods of the present invention.

The methods of the present invention result in the reduction in serum levels of one or more lipid component selected from the group consisting of LDL-C, ApoB100, non-HDL-C, total cholesterol, VLDL-C, triglycerides, Lp(a) and remnant cholesterol. For example, according to certain embodiments of the present invention, administration of a pharmaceutical composition comprising a PCSK9 inhibitor to a subject with a PCSK9 GOFm, results in a mean percent reduction from baseline in serum low density lipoprotein cholesterol (LDL-C) of at least about 25%, 30%, 40%, 50%, 60%, or greater; a mean percent reduction from baseline in ApoB100 of at least about 25%, 30%, 40%, 50%, 60%, or greater; a mean percent reduction from baseline in non-HDL-C of at least about 25%, 30%, 40%, 50%, 60%, or greater; a mean percent reduction from baseline in total cholesterol of at least about 10%, 15%, 20%, 25%, 30%, 35%, or greater; a mean percent reduction from baseline in VLDL-C of at least about 5%, 10%, 15%, 20%, 25%, 30%, or greater; a mean percent reduction from baseline in triglycerides of at least about 5%, 10%, 15%, 20%, 25%, 30%, 35% or greater; and/or a mean percent reduction from baseline in Lp(a) of at least about 5%, 10%, 15%, 20%, 25%, or greater.

### PCSK9 Inhibitors

The methods of the present invention comprise administering to a patient a therapeutic composition comprising a PCSK9 inhibitor. As used herein, a “PCSK9 inhibitor” is any agent which binds to or interacts with human PCSK9 and inhibits the normal biological function of PCSK9 in vitro or in vivo. Non-limiting examples of categories of PCSK9 inhibitors include small molecule PCSK9 antagonists, peptide-based PCSK9 antagonists (e.g., “peptibody” molecules), and antibodies or antigen-binding fragments of antibodies that specifically bind human PCSK9.

The term “human proprotein convertase subtilisin/kexin type 9” or “human PCSK9” or “hPCSK9”, as used herein, refers to PCSK9 having the nucleic acid sequence shown in SEQ ID NO:754 and the amino acid sequence of SEQ ID NO:755, or a biologically active fragment thereof.

The term “antibody”, as used herein, is intended to refer to immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-PCSK9 antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (Xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, may be adapted for use in the context of an antigen-binding fragment of an antibody of the present invention using routine techniques available in the art.

The constant region of an antibody is important in the ability of an antibody to fix complement and mediate cell-dependent cytotoxicity. Thus, the isotype of an antibody may be selected on the basis of whether it is desirable for the antibody to mediate cytotoxicity.

The term “human antibody”, as used herein, is intended to include antibodies having variable and constant regions derived from human germline immunoglobulin sequences. The human antibodies of the invention may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification.

The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30:105) to levels typically observed using a human IgG1 hinge. The instant invention encompasses antibodies having one or more mutations in the hinge, C_{H}2 or C_{H}3 region which may be desirable, for example, in production, to improve the yield of the desired antibody form.

An “isolated antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present invention. An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” PCSK9, as used in the context of the present invention, includes antibodies that bind PCSK9 or portion thereof with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human PCSK9, however, have cross-reactivity to other antigens, such as PCSK9 molecules from other (non-human) species.

The anti-PCSK9 antibodies useful for the methods of the present invention may comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present invention includes methods involving the use of antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. The use of antibodies and antigen-binding fragments obtained in this general manner are encompassed within the present invention.

The present invention also includes methods involving the use of anti-PCSK9 antibodies comprising variants of any of the HCVR, LCVR, and/or CDR amino acid sequences disclosed herein having one or more conservative substitutions. For example, the present invention includes the use of anti-PCSK9 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “surface plasmon resonance”, as used herein, refers to an optical phenomenon that allows for the analysis of real-time interactions by detection of alterations in protein concentrations within a biosensor matrix, for example using the BIAcore™ system (Biacore Life Sciences division of GE Healthcare, Piscataway, N.J.).

The term “K_{D}”, as used herein, is intended to refer to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

According to certain embodiments, the anti-PCSK9 antibody used in the methods of the present invention is an antibody with pH-dependent binding characteristics. As used herein, the expression “pH-dependent binding” means that the antibody or antigen-binding fragment thereof exhibits “reduced binding to PCSK9 at acidic pH as compared to neutral pH” (for purposes of the present disclosure, both expressions may be used interchangeably). For the example, antibodies “with pH-dependent binding characteristics” includes antibodies and antigen-binding fragments thereof that bind PCSK9 with higher affinity at neutral pH than at acidic pH. In certain embodiments, the antibodies and antigen-binding fragments of the present invention bind PCSK9 with at least 3, 5, 10, 15, 20, 25, 30, 35, 40, 45, 50, 55, 60, 65, 70, 75, 80, 85, 90, 95, 100, or more times higher affinity at neutral pH than at acidic pH.

According to this aspect of the invention, the anti-PCSK9 antibodies with pH-dependent binding characteristics may possess one or more amino acid variations relative to the parental anti-PCSK9 antibody. For example, an anti-PCSK9 antibody with pH-dependent binding characteristics may contain one or more histidine substitutions or insertions, e.g., in one or more CDRs of a parental anti-PCSK9 antibody. Thus, according to certain embodiments of the present invention, methods are provided comprising administering an anti-PCSK9 antibody which comprises CDR amino acid sequences (e.g., heavy and light chain CDRs) which are identical to the CDR amino acid sequences of a parental anti-PCSK9 antibody, except for the substitution of one or more amino acids of one or more CDRs of the parental antibody with a histidine residue. The anti-PCSK9 antibodies with pH-dependent binding may possess, e.g., 1, 2, 3, 4, 5, 6, 7, 8, 9, or more histidine substitutions, either within a single CDR of a parental antibody or distributed throughout multiple (e.g., 2, 3, 4, 5, or 6) CDRs of a parental anti-PCSK9 antibody. For example, the present invention includes the use of anti-PCSK9 antibodies with pH-dependent binding comprising one or more histidine substitutions in HCDR1, one or more histidine substitutions in HCDR2, one or more histidine substitutions in HCDR3, one or more histidine substitutions in LCDR1, one or more histidine substitutions in LCDR2, and/or one or more histidine substitutions in LCDR3, of a parental anti-PCSK9 antibody.

As used herein, the expression “acidic pH” means a pH of 6.0 or less (e.g., less than about 6.0, less than about 5.5, less than about 5.0, etc.). The expression “acidic pH” includes pH values of about 6.0, 5.95, 5.90, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

### Preparation of Human Antibodies

Methods for generating human antibodies in transgenic mice are known in the art. Any such known methods can be used in the context of the present invention to make human antibodies that specifically bind to human PCSK9.

Using VELOCIMMUNE™ technology (see, for example, U.S. Pat. No. 6,596,541, Regeneron Pharmaceuticals) or any other known method for generating monoclonal antibodies, high affinity chimeric antibodies to PCSK9 are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc, using standard procedures known to those skilled in the art. The mouse constant regions are replaced with a desired human constant region to generate the fully human antibody of the invention, for example wild-type or modified IgG1 or IgG4. While the constant region selected may vary according to specific use, high affinity antigen-binding and target specificity characteristics reside in the variable region.

In general, the antibodies that can be used in the methods of the present invention possess high affinities, as described above, when measured by binding to antigen either immobilized on solid phase or in solution phase. The mouse constant regions are replaced with desired human constant regions to generate the fully human antibodies of the invention. While the constant region selected may vary according to specific use, high affinity antigen-binding and target specificity characteristics reside in the variable region.

Specific examples of human antibodies or antigen-binding fragments of antibodies that specifically bind PCSK9 which can be used in the context of the methods of the present invention include any antibody or antigen-binding fragment which comprises the three heavy chain CDRs (HCDR1, HCDR2 and HCDR3) contained within a heavy chain variable region (HCVR) having an amino acid sequence selected from the group consisting of SEQ ID NOs: 2, 18, 22, 26, 42, 46, 50, 66, 70, 74, 90, 94, 98, 114, 118, 122, 138, 142, 146, 162, 166, 170, 186, 190, 194, 210, 214, 218, 234, 238, 242, 258, 262, 266, 282, 286, 290, 306, 310, 314, 330, 334, 338, 354, 358, 362, 378, 382, 386, 402, 406, 410, 426, 430, 434, 450, 454, 458, 474, 478, 482, 498, 502, 506, 522, 526, 530, 546, 550, 554, 570, 574, 578, 594, 598, 602, 618, 622, 626, 642, 646, 650, 666, 670, 674, 690, 694, 698, 714, 718, 722, 738 and 742, or a substantially similar sequence thereof having at least 90%, at least 95%, at least 98% or at least 99% sequence identity. The antibody or antigen-binding fragment may comprise the three light chain CDRs (LCVR1, LCVR2, LCVR3) contained within a light chain variable region (LCVR) having an amino acid sequence selected from the group consisting of SEQ ID NOs: 10, 20, 24, 34, 44, 48, 58, 68, 72, 82, 92, 96, 106, 116, 120, 130, 140, 144, 154, 164, 168, 178, 188, 192, 202, 212, 216, 226, 236, 240, 250, 260, 264, 274, 284, 288, 298, 308, 312, 322, 332, 336, 346, 356, 360, 370, 380, 384, 394, 404, 408, 418, 428, 432, 442, 452, 456, 466, 476, 480, 490, 500, 504, 514, 524, 528, 538, 548, 552, 562, 572, 576, 586, 596, 600, 610, 620, 624, 634, 644, 648, 658, 668, 672, 682, 692, 696, 706, 716, 720, 730, 740 and 744, or a substantially similar sequence thereof having at least 90%, at least 95%, at least 98% or at least 99% sequence identity.

In certain embodiments of the present invention, the antibody or antigen-binding fragment thereof comprises the six CDRs (HCDR1, HCDR2, HCDR3, LCDR1, LCDR2 and LCDR3) from the heavy and light chain variable region amino acid sequence pairs (HCVR/LCVR) selected from the group consisting of SEQ ID NOs: 2/10, 18/20, 22/24, 26/34, 42/44, 46/48, 50/58, 66/68, 70/72, 74/82, 90/92, 94/96, 98/106, 114/116, 118/120, 122/130, 138/140, 142/144, 146/154, 162/164, 166/168, 170/178, 186/188, 190/192, 194/202, 210/212, 214/216, 218/226, 234/236, 238/240, 242/250, 258/260, 262/264, 266/274, 282/284, 286/288, 290/298, 306/308, 310/312, 314/322, 330/332, 334/336, 338/346, 354/356, 358/360, 362/370, 378/380, 382/384, 386/394, 402/404, 406/408, 410/418, 426/428, 430/432, 434/442, 450/452, 454/456, 458/466, 474/476, 478/480, 482/490, 498/500, 502/504, 506/514, 522/524, 526/528, 530/538, 546/548, 550/552, 554/562, 570/572, 574/576, 578/586, 594/596, 598/600, 602/610, 618/620, 622/624, 626/634, 642/644, 646/648, 650/658, 666/668, 670/672, 674/682, 690/692, 694/696, 698/706, 714/716, 718/720, 722/730, 738/740 and 742/744.

In certain embodiments of the present invention, the anti-PCSK9 antibody, or antigen-binding fragment thereof, that can be used in the methods of the present invention has HCDR1/HCDR2/HCDR3/LCDR1/LCDR2/LCDR3 amino acid sequences selected from SEQ ID NOs: 76/78/80/84/86/88 (mAb316P) and 220/222/224/228/230/232 (mAb300N) (See US Patent App. Publ No. 2010/0166768).

In certain embodiments of the present invention, the antibody or antigen-binding fragment thereof comprises HCVR/LCVR amino acid sequence pairs selected from the group consisting of SEQ ID NOs: 2/10, 18/20, 22/24, 26/34, 42/44, 46/48, 50/58, 66/68, 70/72, 74/82, 90/92, 94/96, 98/106, 114/116, 118/120, 122/130, 138/140, 142/144, 146/154, 162/164, 166/168, 170/178, 186/188, 190/192, 194/202, 210/212, 214/216, 218/226, 234/236, 238/240, 242/250, 258/260, 262/264, 266/274, 282/284, 286/288, 290/298, 306/308, 310/312, 314/322, 330/332, 334/336, 338/346, 354/356, 358/360, 362/370, 378/380, 382/384, 386/394, 402/404, 406/408, 410/418, 426/428, 430/432, 434/442, 450/452, 454/456, 458/466, 474/476, 478/480, 482/490, 498/500, 502/504, 506/514, 522/524, 526/528, 530/538, 546/548, 550/552, 554/562, 570/572, 574/576, 578/586, 594/596, 598/600, 602/610, 618/620, 622/624, 626/634, 642/644, 646/648, 650/658, 666/668, 670/672, 674/682, 690/692, 694/696, 698/706, 714/716, 718/720, 722/730, 738/740 and 742/744.

### Pharmaceutical Compositions and Methods of Administration

The present invention includes methods which comprise administering a PCSK9 inhibitor to a patient, wherein the PCSK9 inhibitor is contained within a pharmaceutical composition. The pharmaceutical compositions of the invention are formulated with suitable carriers, excipients, and other agents that provide suitable transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

Various delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents.

A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park Ill.), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc.

### Dosage

The amount of PCSK9 inhibitor (e.g., anti-PCSK9 antibody) administered to a subject according to the methods of the present invention is, generally, a therapeutically effective amount. As used herein, the phrase “therapeutically effective amount” means a dose of PCSK9 inhibitor that results in a detectable reduction (at least about 5%, 10%, 15%, 20%, 25%, 30%, 35%, 40%, 45%, 50%, 55%, 60%, 65%, 70%, 75%, or more from baseline) in one or more parameters selected from the group consisting of LDL-C, ApoB100, non-HDL-C, total cholesterol, VLDL-C, triglycerides, Lp(a) and remnant cholesterol.

In the case of an anti-PCSK9 antibody, a therapeutically effective amount can be from about 0.05 mg to about 600 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 75 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-PCSK9 antibody.

The amount of anti-PCSK9 antibody contained within the individual doses may be expressed in terms of milligrams of antibody per kilogram of patient body weight (i.e., mg/kg). For example, the anti-PCSK9 antibody may be administered to a patient at a dose of about 0.0001 to about 10 mg/kg of patient body weight.

### Combination Therapies

The methods of the present invention, according to certain embodiments, may comprise administering a pharmaceutical composition comprising an anti-PCSK9 antibody to a patient who is on a therapeutic regimen for the treatment of hypercholesterolemia at the time of, or just prior to, administration of the pharmaceutical composition of the invention. For example, a patient who has previously been diagnosed with hypercholesterolemia may have been prescribed and is taking a stable therapeutic regimen of another drug prior to and/or concurrent with administration of a pharmaceutical composition comprising an anti-PCSK9 antibody. The prior or concurrent therapeutic regimen may comprise, e.g., (1) an agent which induces a cellular depletion of cholesterol synthesis by inhibiting 3-hydroxy-3-methylglutaryl (HMG)-coenzyme A (CoA) reductase, such as a statin (e.g., cerivastatin, atorvastatin, simvastatin, pitavastatin, rosuvastatin, fluvastatin, lovastatin, pravastatin, etc.); (2) an agent which inhibits cholesterol uptake and or bile acid re-absorption; (3) an agent which increase lipoprotein catabolism (such as niacin); and/or (4) activators of the LXR transcription factor that plays a role in cholesterol elimination such as 22-hydroxycholesterol. In certain embodiments, the patient, prior to or concurrent with administration of an anti-PCSK9 antibody is on a fixed combination of therapeutic agents such as ezetimibe plus simvastatin; a statin with a bile resin (e.g., cholestyramine, colestipol, colesevelam); niacin plus a statin (e.g., niacin with lovastatin); or with other lipid lowering agents such as omega-3-fatty acid ethyl esters (for example, omacor).

### Administration Regimens

According to certain embodiments of the present invention, multiple doses of a PCSK9 inhibitor (i.e., a pharmaceutical composition comprising a PCSK9 inhibitor) may be administered to a subject over a defined time course. The methods according to this aspect of the invention comprise sequentially administering to a subject multiple doses of a PCSK9 inhibitor. As used herein, “sequentially administering” means that each dose of PCSK9 inhibitor is administered to the subject at a different point in time, e.g., on different days separated by a predetermined interval (e.g., hours, days, weeks or months). The present invention includes methods which comprise sequentially administering to the patient a single initial dose of a PCSK9 inhibitor, followed by one or more secondary doses of the PCSK9 inhibitor, and optionally followed by one or more tertiary doses of the PCSK9 inhibitor.

The terms “initial dose,” “secondary doses,” and “tertiary doses,” refer to the temporal sequence of administration of the individual doses of a pharmaceutical composition comprising a PCSK9 inhibitor. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the PCSK9 inhibitor, but generally may differ from one another in terms of frequency of administration. In certain embodiments, however, the amount of PCSK9 inhibitor contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

According to exemplary embodiments of the present invention, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of antigen-binding molecule which is administered to a patient prior to the administration of the very next dose in the sequence with no intervening doses.

The methods according to this aspect of the invention may comprise administering to a patient any number of secondary and/or tertiary doses of a PCSK9 inhibitor. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2, 4, 6, 8 or more weeks after the immediately preceding dose. Similarly, in embodiments involving multiple tertiary doses, each tertiary dose may be administered at the same frequency as the other tertiary doses. For example, each tertiary dose may be administered to the patient 1 to 2, 4, 6, 8 or more weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

The present invention includes administration regimens comprising an up-titration option (also referred to herein as “dose modification”). As used herein, an “up-titration option” means that, after receiving a particular number of doses of a PCSK9 inhibitor, if a patient has not achieved a specified reduction in one or more defined therapeutic parameters, the dose of the PCSK9 inhibitor is thereafter increased. For example, in the case of a therapeutic regimen comprising administration of 75 mg doses of an anti-PCSK9 antibody to a patient at a frequency of once every two weeks, if after 8 weeks (i.e., 5 doses administered at Week 0, Week 2 and Week 4, Week 6 and Week 8), the patient has not achieved a serum LDL-C concentration of less than 70 mg/dL, then the dose of anti-PCSK9 antibody is increased to e.g., 150 mg administered once every two weeks thereafter (e.g., starting at Week 10 or Week 12, or later).

### Cascade Screening Methods

The present invention also includes cascade screening methods to identify subjects having, or at risk of developing autosomal dominant hypercholesterolemia (ADH). “Cascade screening,” as used herein, means identifying an individual having, or at risk of developing a genetic condition by a process of systematic family tracing of a particular genetic mutation. (See Ned and Sijbrands, PLoS Curr. 2011, 3:RRN1238). The methods according to this aspect of the invention comprise: (a) identifying a first individual with ADH who carries a particular PCSK9-GOFm (aka an “index patient”); and (b) screening biologically-related family members of the index patient for the presence of the PCSK9-GOFm. The PCSK9-GOFm can be any gain-of-function mutation in PCSK9 that is associated with ADH, e.g., V4I, E32K, D35Y, E48K, P71L, R96C, L108R, S127R, D129N, R215H, F216L, R218S, R357H, D374H, D374Y, S465L or R496W.

As used herein, a “biologically-related family member” includes any person sharing a common ancestor with the first individual, including, e.g., first-, second-, third-, fourth-, etc. degree relatives. The methods of the present invention may comprise additional screening steps to identify individuals at risk for ADH, including, e.g., measuring levels of serum LDL-C, HDL-C, non-HDL-C, VLDL-C, Apo B100, Apo A1, triglycerides, Lp(a), and combinations thereof. Standard genetic tests and DNA sequencing methodologies can be used to screen for a PCSK9-GOFm.

According to the methods of this aspect of the invention, if a family member possesses the PCSK9-GOFm that was originally identified in the index patient, then it can be concluded that the family member has or is at risk of developing ADH.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Generation of Human Antibodies to Human PCSK9

Human anti-PCSK9 antibodies were generated as described in U.S. Pat. No. 8,062,640. The exemplary PCSK9 inhibitor used in the following Example is the human anti-PCSK9 antibody designated “mAb316P” (also known as alirocumab). mAb316P has the following amino acid sequence characteristics: heavy chain variable region (HCVR) comprising SEQ ID NO:90; light chain variable domain (LCVR) comprising SEQ ID NO:92; heavy chain complementarity determining region 1 (HCDR1) comprising SEQ ID NO:76; HCDR2 comprising SEQ ID NO:78; HCDR3 comprising SEQ ID NO:80; light chain complementarity determining region 1 (LCDR1) comprising SEQ ID NO:84; LCDR2 comprising SEQ ID NO:86; and LCDR3 comprising SEQ ID NO:88.

### Example 2

### Identification and Characterization of Patients with Autosomal Dominant Hypercholesterolemia Caused by Gain-of-Function Mutations in Proprotein Convertase Subtilisin/Kexin Type 9 (PCSK9), and Comparison with Patients with Familial Hypercholesterolemia (FH) and Familial Defective Apolipoprotein B (FDB)

Autosomal Dominant Hypercholesterolemia (ADH) is a common disorder of lipid metabolism characterized by high levels of serum LDL cholesterol (LDL-C), and early onset cardiovascular disease. (Robinson, J. Manag. Care Pharm. 2013, 19:139-149). The most frequent mutations causing ADH are found in the LDL receptor (LDLR; familial hypercholesterolemia [FH]) or its ligand apolipoprotein B (ApoB; familial defective ApoB [FDB]). ADH can also be caused by gain-of-function mutations in PCSK9 (“PCSK9-GOFm). (Seidah et al., Proc. Natl. Acad. Sci. USA. 2003, 100:928-933). PCSK9 GOFm appear in ˜1% of patients with ADH. To date, relatively few patients with PCSK9 GOFm have been described, and their clinical syndrome is incompletely characterized.

To better understand the geographic and familial distribution of PCSK9 GOFm, their clinical manifestations, and their comparison to FH and FDB a retrospective, cross-sectional parallel-group observational cohort study was conducted. A global survey of potential sites was initiated, with 200 sites contacted in total; 180 sites responded, with 16 positive responses. There were 12 active contributing sites from eight countries (France, Japan, Norway, Portugal, South Africa, The Netherlands, the UK and the USA). Data collected included patient demographics, genotype (PCSK9, LDLR, ApoB, ApoE), baseline and on-treatment lipid profiles, lipid lowering therapies (LLTs), presence of xanthoma, xanthelasma, and corneal arcus, and the occurrence and age of onset of CVD. Patients with PCSK9 GOFm confirmed by molecular testing (DNA sequencing) were matched for age and sex with patients with molecularly proven FH (N=2126) and FDB (N=470) from the Dutch registry. The LDLR mutations were characterized as “defective” (missense, small in-frame indel, synonymous with added splice site) or “deficient” (large or frame-shifting indel, nonsense, splice site, promoter mutations) and compared their lipid profiles.

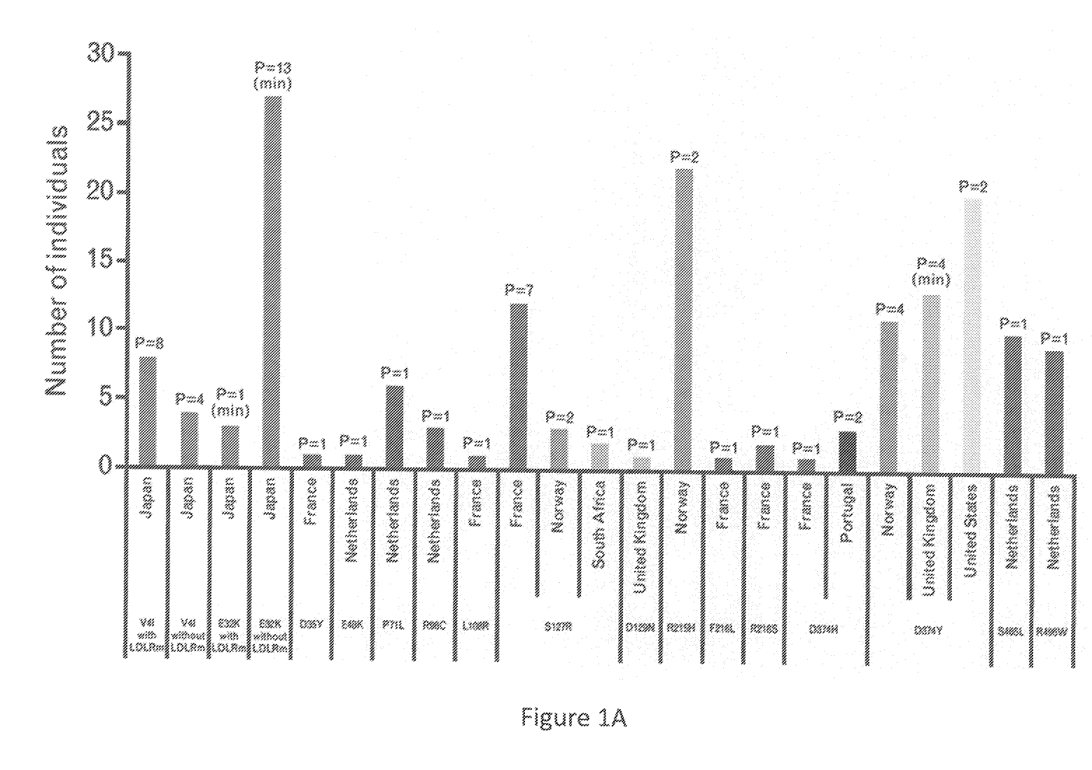

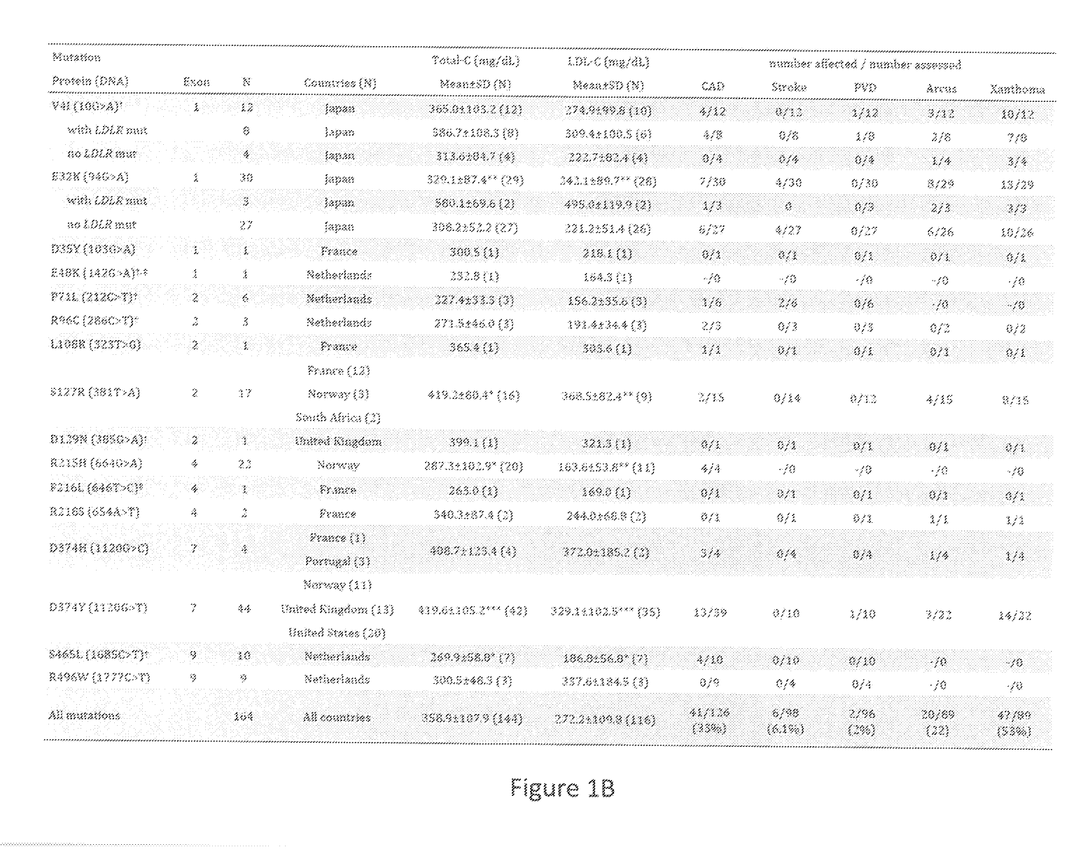

For the present study, 200 lipid specialty centers around the world were contacted and 164 patients (83 men, 81 women) with PCSK9 GOF mutations were identified from 12 centers in France, Japan, Norway, Portugal, South Africa, The Netherlands, the UK, and the USA. Patients carried 16 different missense mutations, 6 of which were previously undescribed. The six previously unreported GOFm are: V4I, E48K, P71L, R96C, D129N and S465L. Individual PCSK9 GOF mutations generally had restricted geographic distributions and were found in a small number of pedigrees. (FIGS. 1A and 1B). Examples include 22 patients with R215H found only in 2 pedigrees in Norway, and 12 patients with V4I and 30 patients with E32K found only in Japan. (FIG. 1). For pooled patients with PCSK9 GOFm, mean untreated total and LDL cholesterol were 359 and 272 mg/dL, respectively. Eleven patients were double heterozygotes for mutations in PCSK9 and LDLR; these patients tended to have higher untreated lipids compared to patients with the same GOF mutation alone, as previously reported for the three E32K double heterozygote patients.

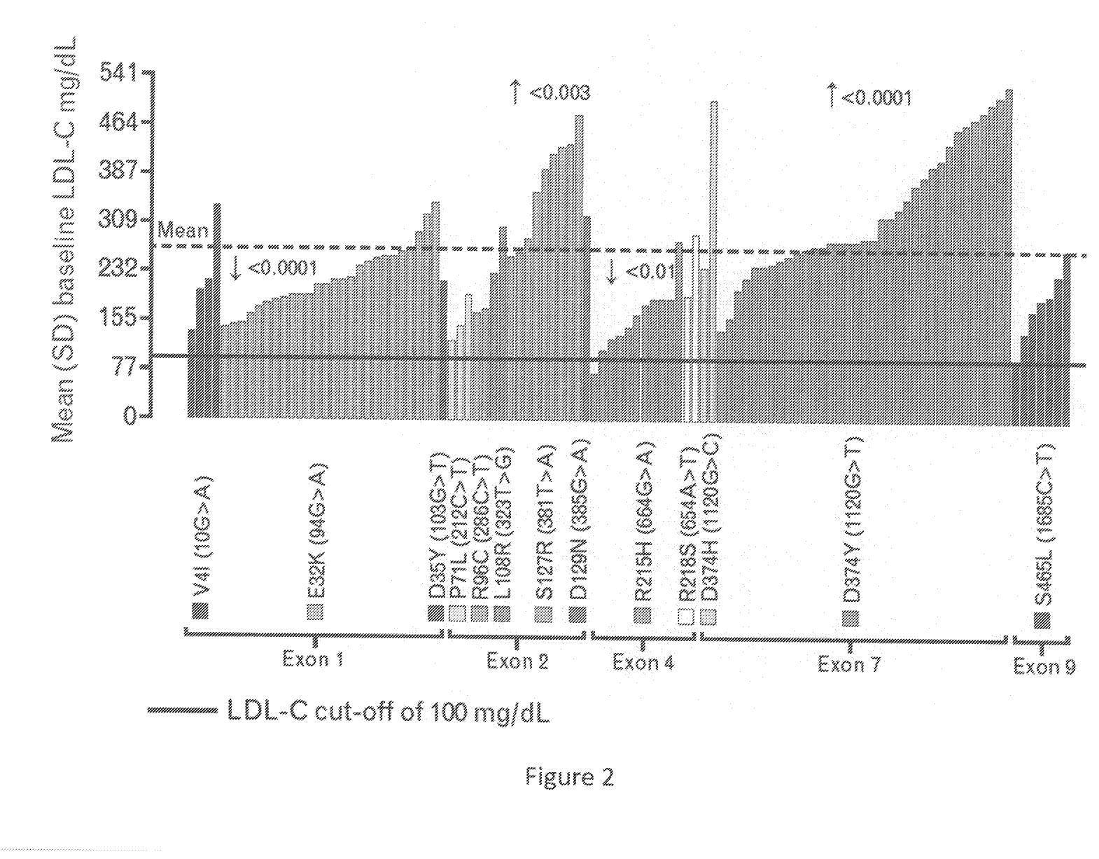

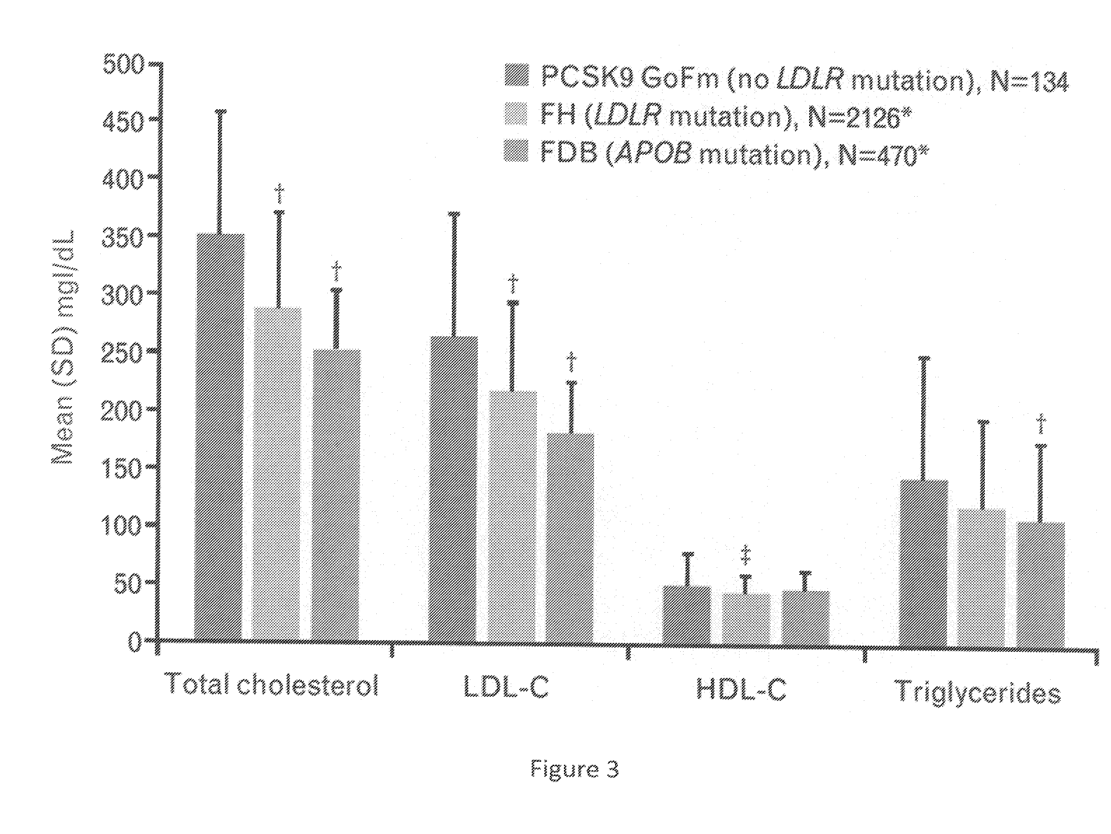

GOF mutations were found in all structural protein domains and 5 of 9 coding exons (FIG. 2), and were associated with varying degrees of lipid abnormalities (FIG. 1B). In general, lipid abnormalities were more severe in patients with PCSK9 GOFm compared to those with FH and FDB; mean untreated LDL-C was highest in patients with PCSK9 GOFm and lowest with FDB (FIG. 3). Untreated lipid levels associated with each mutation were compared to the entire population of patients with GOFm: D374Y and S127R carriers had severe lipid abnormalities, while E32K, R215H and S465L carriers were comparatively mild, although substantial variation was present in patients carrying the same mutation.

Of the patients with PCSK9 for whom data were available, 53% had evidence of xanthoma, 15% had evidence of xanthelasmata, and 22% had evidence of arcus cornealis; and 33% had a history of coronary artery disease; mean onset was 49.4 (13.8) years. Clinical presentation and CVD burden in patients with PCSK9 GOFm are summarized in Table 1A.

TABLE 1A
Clinical Presentation and CVD Burden in Patients with PCSK9 GOFm

| | Current Study | Published Literature | |
|---|---|---|---|
| Cardiovascular Disease | | | |
| Findings, % (N) | PCSK9 GOFm | FH^{a} | FDB^{b} |
| Arterial events | 33% (126) | 33% (1940) | 37% (516) |
| Age of onset, yrs ± SD | 49.4 ± 13.8 | 48.2 | 46.6 ± 9.8 |
| Peripheral events | 2% (96) | — | 21% (516) |
| Age of onset, yrs | 62 | — | — |
| Neural events | 6% (98) | — | — |
| Age of onset, yrs ± SD | 60.0 ± 8.4 | | |
| Reported physical | | | |
| manifestations (% patients) | PCSK9 GOFm | FH^{c} | FDB^{d} |
| Xanthoma | 53% | 44% | 36% |
| Xanthelasmata | 15% | — | — |
| Arcus cornealis | 22% | 31% | 38% |

^{a}Jansen et al., Arterioscler Thromb Vasc Biol. 2005, 25: 1475-1481.
^{b}Fouchier et al., Semin Vasc Med. 2004, 4: 259-264.
^{c}de Sauvage Nolting et al., J Intern Med. 2003, 253: 161-168.
^{d}Hanson et al., Arterioscler Thromb Vasc Biol. 1997, 17: 741-747.

The physical stigmata of elevated cholesterol were frequent among patients with PCSK9 GOFm (FIG. 2), with prevalence similar to FH and FDB (familial defective Apolipoprotein B) as previously reported (Table 1A). Also similar to FH and FDB, 44% of patients with PCSK9 GOFm had a history of cardiovascular disease. Coronary artery disease (CAD) was the most prevalent manifestation (33%) with an average age of onset of 49.4±13.8 years (FIG. 2 and Table 1A).

In a comparison between patients with PCSK9 GOFm and FH and FDB patients drawn from the Dutch Hypercholesterolemia Registry, patients with PCSK9 GOFm had the highest, and FDB patients the lowest mean untreated LDL cholesterol levels (Table 1B). Among patients with FH, those with deficient mutations had higher untreated LDL cholesterol levels than those with defective mutations (Table 1B). Although lipid-lowering therapy (primarily statins) improved lipid profiles in patients with PCSK9 GOFm, a substantial proportion failed to achieve guideline LDL cholesterol levels.

TABLE 1B
Comparison of Untreated Lipid Profiles (means ± SD) of Familial Gain-of-Function
Mutation in PCSK9 (FGP), Familial Defective Apolipoprotein B (FDB) and Defective and
Deficient LDLR Mutations in familial hypercholesterolemia (FH).

| | | | FH by LDLR mutation class | | |
|---|---|---|---|---|---|
| | PCSK9 GOFm | | All FH | Defective LDLR | Deficient LDLR |
| | (N) | FDB (N = 470) | (N = 2126) | (N = 1398) | (N = 728) |
| Age (yr) | 36.7 ± 18.6 | 32.1 ± 16.9 | 28.1 ± 16.5 | 29.2 ± 16.4 | 26.1 ± 16.5 |
| | (135) | | | | |
| Total-C | 351.9 ± 104.4 | 254.8 ± 50.7*** | 290.0 ± 82.8*** | 277.3 ± 74.2*** | 314.8 ± 92.8 |
| (mg/dL | (134) | | | | |
| LDL-C | 266.8 ± 108.3 | 184.8 ± 43.3*** | 219.6 ± 76.6*** | 206.5 ± 67.3*** | 245.2 ± 86.2 |
| (mg/dL) | (108) | | | | |
| HDL-C | 54.2 ± 27.1 | 48.7 ± 16.2 | 46.4 ± 14.3** | 46.8 ± 15.1** | 45.2 ± 13.1*** |
| (mg/dL | (108) | | | | |
| TG | 150.6 ± 115.1 | 111.6 ± 65.5** | 121.3 ± 76.2 | 122.2 ± 77.1* | 120.5 ± 75.3 |
| (mg/dL) | (108) | | | | |

*P < 0.05;
**P < 0.01;
***P < 0.001 when compared to PCSK9 GOF mutation carriers. The eleven patients who were double heterozygotes for mutations in PCSK9 and LDLR were excluded from the analysis.

Index patients with either FH or FDB had higher baseline LDLC levels than family members and patients with deficient LDLR mutations had higher baseline LDLC than those with defective mutations.

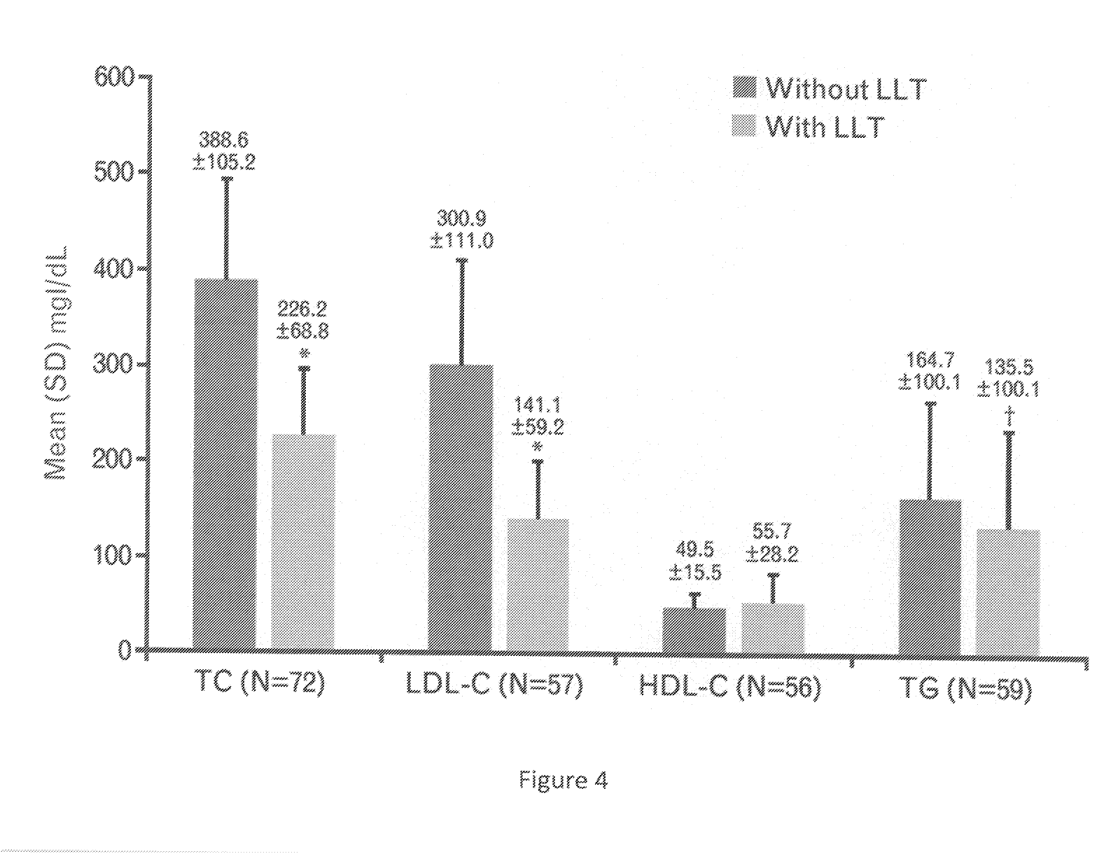

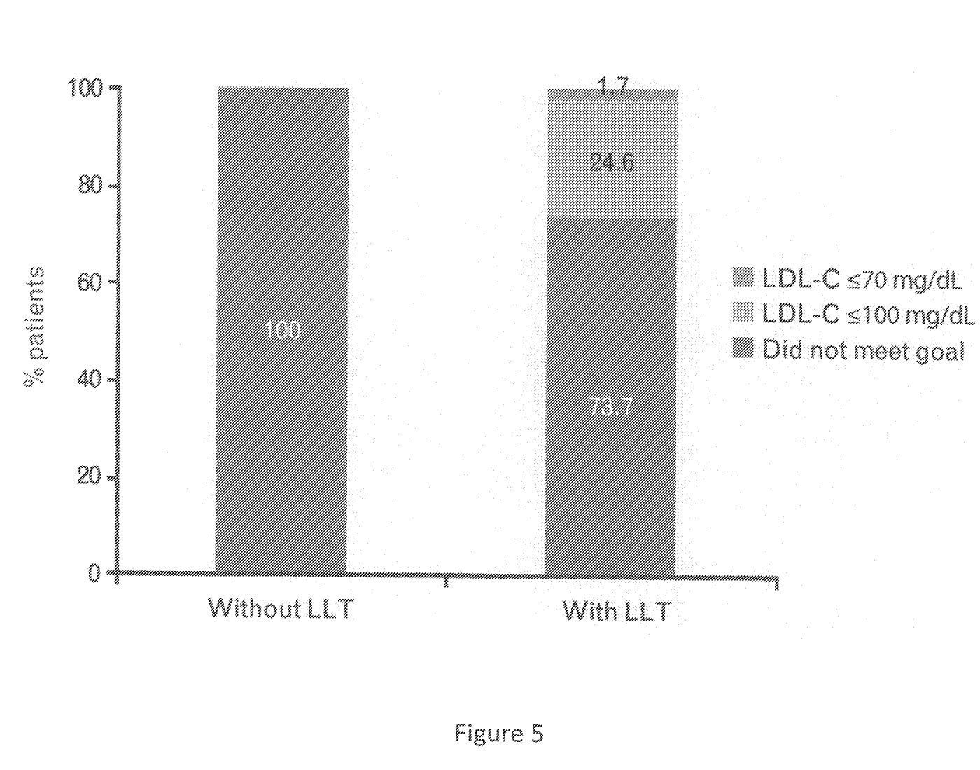

Although lipid profiles may be improved by utilizing existing LTTs (e.g., statins, ezetimibe) in patients with PCSK9 GOFm (FIG. 4), over 70% of patients do not achieve a LDL-C goal of <100 mg/dL, with fewer than 2% achieving <70 mg/dL (FIG. 5).

### Conclusions

PCSK9 GOFm causes a severe form of ADH, and most patients fail to attain target LDL-C on current therapy. The present observational study demonstrated that PCSK9 GOF variants had mostly restricted geographical distributions, were found in a limited number of pedigrees, and exhibited significant phenotypic variability in associated disease severity. While GOF mutations were found throughout the PCSK9 coding sequence, the present study confirms that carriers of either D374Y or S127R mutations had significantly higher untreated LDL cholesterol levels than the mean of all other PCSK9 GOF mutation carriers. The geographic isolation of GOF variants suggests they are likely due to private mutations in different populations. In the present study, extensive efforts were undertaken to define the genetic architecture of ADH in Holland and Japan, and no overlap in the variants were found, supporting the geographical isolation of PCSK9 GOF mutations.

Despite variability in disease severity of individual mutations, pooled analyses of patients with PCSK9 GOFm revealed significantly greater LDL cholesterol levels as compared to patients with FH or FDB. FH patients are found worldwide, and LDLR variants causing FH are distributed throughout the gene (over 1600 reported) with greater disease severity associated with individual mutations. In contrast, FDB is also found worldwide, though the ApoB variant R3527Q, found primarily in northern Europeans, is by far the most common cause (>95%). For the present study, the patients with PCSK9 GOFm were matched with FH and FDB patients from the Dutch Familial Hypercholesterolemia Registry, the largest such resource in the world. Based on the results of the present study, it is concluded that the severity of the PCSK9 GOFm phenotype warrants aggressive lipid-lowering therapies in these patients.

Finally, the results set forth herein suggest that cascade screening would be an effective methods for identifying additional affected family members once an index patient (i.e., a patient with ADH harboring a particular PCSK9 GOFm) is identified.

### Example 3

### Clinical Trial of an Anti-PCSK9 Monoclonal Antibody in Patients with Autosomal Dominant Hypercholesterolemia and Gain-of-Function Mutations in PCSK9

### Introduction

A phase 2 clinical trial was conducted to evaluate the pharmacodynamics and safety of an anti-PCSK9 antibody in patients with autosomal dominant hypercholesterolemia (ADH) and gain-of-function mutations in one or both alleles of the PCSK9 gene (PCSK9-GOFm). The anti-PCSK9 antibody used in this study is a fully human monoclonal antibody referred to herein as mAb316P (also referred to as alirocumab). the primary objective of the present study was to assess the effect of mAb316P on serum low density lipoprotein cholesterol (LDL-C) during 14 weeks of subcutaneously (SC) administered mAb316P in patients with ADH and PCSK9-GOFm. Secondary objectives of the study were to assess in patients with PCSK9-GOFm: (a) the safety and tolerability of SC administered mAb316P; (b) the pharmacodynamic effect of mAb316P on other serum lipids/apolipoproteins (Apo) including: total cholesterol, high-density lipoprotein cholesterol (HDL-C), non-HDL-C, very low-density lipoprotein cholesterol (VLDL-C), triglycerides (TG), ApoB100, ApoA1, and lipoprotein (a) (Lp[a]); (c) the pharmacokinetic (PK) profile of multiple SC doses of mAb316P; and (d) the immunogenicity profile in patients after receiving mAb316P every two weeks (q2w).

### Patient Selection

The target population for this study was men and women with ADH and PCSK9 GOFm, ages 18 to 70 inclusive, who had serum LDL-C level ≧70 mg/dL (×0.0259 mmol/L) at screening on a lipid lowering therapy regimen stable for at least 28 days, and who were considered to be not at goal by the investigator. Patients with GOF mutations were defined as patients who, by previous DNA analysis, were shown to carry 1 or more copies of a mutant PCSK9 gene encoding the D374Y, S127R, F216L, R357H, or R218S PCSK9 protein variants. Patient with other PCSK9 alleles that were deemed to result in a GOF phenotype were also considered for inclusion in the study. A total of 13 patients were enrolled and dosed in the study.

The inclusion criteria for the study were as follows: (1) Man or woman between the ages of 18 and 70 years, inclusive; (2) A history of molecularly confirmed PCSK9 GOFm; (3) Plasma LDL-C levels ≧70 mg/dL (×0.0259 mmol/L) at the screening visit (visit 1 [day −28 to −15]) on a lipid lowering therapy regimen stable for at least 28 days; it was required that LDL-C was considered to be not at goal by the investigator. The possible lipid lowering therapy regimens included, but were not limited to: (i) Statins, (ii) Ezetimibe, (iii) Fibrates, (iv) Niacin, (v) Omega-3 fatty acids, and (vi) Bile acid resins; (4) Body mass index ≧18.0 and ≦40.0 kg/m2 at the screening visit (visit 1 [day −28 to −15]); (5) Systolic blood pressure (BP) ≦150 mm Hg and diastolic BP 595 mm Hg at the screening visit (visit 1 [day −28 to −15]); (6) Willing to refrain from the consumption of no more than 2 standard alcoholic drinks in any 24-hour period for the duration of the study. A standard alcoholic drink was regarded as the equivalent of: 12 ounces beer, 5 ounces of wine, or 1.5 ounces of hard liquor; (7) Willing to refrain from the consumption of alcohol for 24 hours before each study visit; (8) Willing to maintain their usual stable diet and exercise regimen throughout the study; (9) Willing and able to comply with clinic visits and study-related procedures; and (10) Provide signed informed consent.

The exclusion criteria for the study were as follows: (1) Serum triglycerides >350 mg/dL (×0.01129 mmol/L) at the screening visit (visit 1 [day −28 to day −15]) measured after an 8 to 12 hour fast; (2) History of heart failure (New York Heart Association Class II-IV) within the 12 months before the screening visit (visit 1 [day −28 to −15]); (3) History of myocardial infarction, acute coronary syndrome, unstable angina pectoris, stroke, peripheral vascular disease, transient ischemic attack, or cardiac revascularication within the 6 months before the screening visit (visit 1 [day −28 to −15]); (4) History of uncontrolled, clinically significant cardiac dysrhythmias or clinically significant recent changes in ECG 6 months before the screening visit (visit 1 [day −28 to −15]); (5) Known history of active optic nerve disease; (6) History of undergoing LDL apheresis within 3 months before the screening visit (visit 1 [day −28 to −15]); (7) Uncontrolled diabetes mellitus with hemoglobin A1C (HbA1c) >8.5% at the screening visit (visit 1 [day −28 to −15]); (8) Thyroid stimulating hormone (TSH) >1.5× upper limit of normal (ULN) at the screening visit (visit 1 [day −28 to −15]); (9) Alanine aminotransferase (ALT) or aspartate aminotransferase (AST) >2×ULN at the full screening visit (visit 1 [day −28 to −15]) (1 repeat lab was allowed); (10) Creatine phosphokinase (CPK) >3×ULN at the screening visit (visit 1 [day −28 to −15]) (1 repeat lab was allowed); (11) Known sensitivity to monoclonal antibody therapeutics; (12) Participation in a clinical research study evaluating an investigational drug within 30 days, or at least 5 half-lives of the investigational drug, before the screening visit (visit 1 [day −28 to −15]), whichever was longer; (13) Known to be positive for human immunodeficiency virus (HIV), hepatitis B virus, or hepatitis C virus; (14) History of malignancy of any organ system (other than localized basal cell carcinoma of the skin), treated or untreated, within the past 5 years, regardless of whether there is evidence of local recurrence or metastases; (15) Pregnant or breast-feeding women; (16) Sexually active man or woman of childbearing potential who was unwilling to practice adequate contraception during the study (adequate contraceptive measures include stable use of oral contraceptives or other prescription pharmaceutical contraceptives for 2 or more menstrual cycles prior to screening; intrauterine device; bilateral tubal ligation; vasectomy; condom plus contraceptive sponge, foam, or jelly, or diaphragm plus contraceptive sponge, foam, or jelly); and (17) Any medical or psychiatric condition which, in the opinion of the investigator, would place the patient at risk, interfere with patient's participation in the study or interfere with the interpretation of the study results.

### Investigational Drug and Administration

The investigational drug used in this study was mAb316P, which was supplied in a single-use, 1 mL, pre-filled glass syringe, at a concentration of 150 mg/mL in 10 mM histidine, pH 6.0, 0.2% (w/v) polysorbate 20, and 10% (w/v) sucrose. To ensure that a dose of at least 150 mg of mAb316P was delivered from the syringe, the pre-filled syringe contained the targeted dose plus, on average, a 7% overfill (1+0.07 mL). The reference treatment was placebo that matched the mAb316P; it was prepared in the same formulation as mAb316P without the addition of protein.

All study drug injections during the double-blind treatment period were administered SC in the abdomen. All study drug injections during the open-label treatment period (see below) will be administered SC in the abdomen, thigh, or outer upper arm.

### Study Design

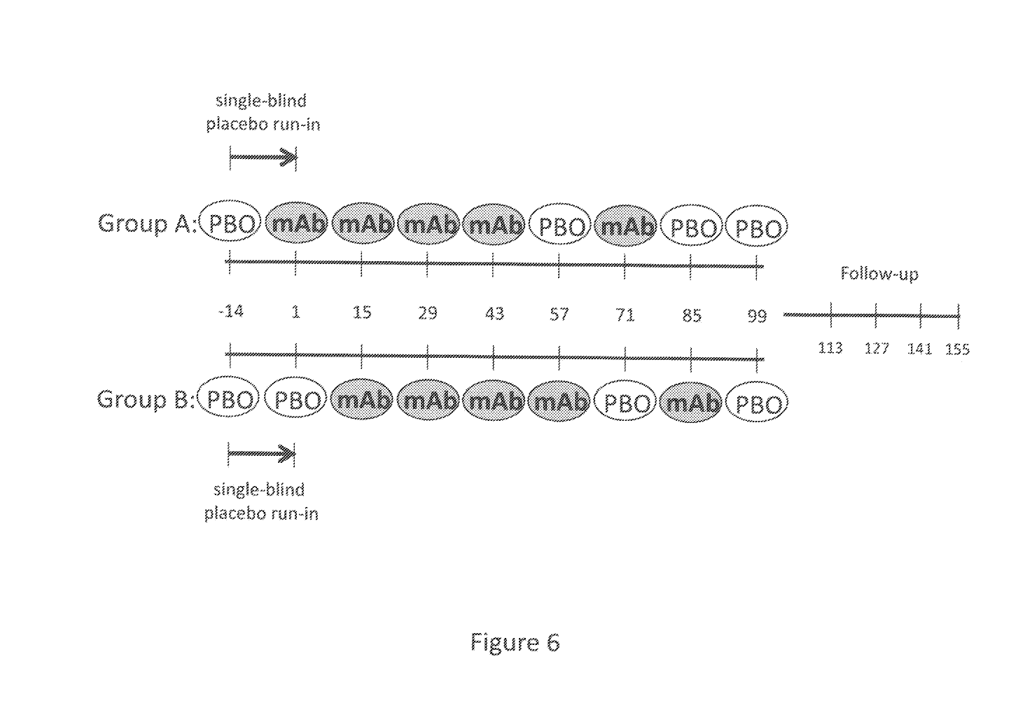

The study design schematic is shown in FIG. 6. The study was designed with a single-blind placebo run-in, double-blind treatment and follow-up periods. The single-blind placebo run-in period (day −14 to day 1) was included to help ensure that patients were stabilized on their daily lipid lowering therapy prior to receiving study drug. As discussed in more detail below, all patients in the study received 5 doses of 150 mg mAb316P SC and 4 doses of matching placebo (FIG. 6). All patients in the study received four bi-weekly doses of mAb316P followed by another dose of mAb316P one month later. After successfully completing the double-blind treatment phase, patients were permitted to enter an open-label treatment period to evaluate the long-term safety and durability of LDL-C lowering of SC administered mAb316P. Patients in the open-label portion of the study will receive 150 mg of mAb316P SC once every two weeks (q2w) for an additional 3 years.

Patients were assessed for study eligibility at the screening visit (visit 1 [day −28 to −15]). Patients on lipid lowering therapy prior to enrollment were required to be on a stable regimen for at least 28 days prior to screening and were required to remain on this regimen through visit 15 (day 155). After visit 15 (day 155), the investigator was permitted to change the patient's lipid lowering therapy if needed.

On day −14, all patients who met the eligibility criteria entered the 2-week, single-blind, placebo run-in phase, and received a dose of placebo administered SC. On study day 1 (baseline), all patients were randomized in an approximate 1:1 ratio to group A (6 patients) or group B (7 patients). On study day 1, all patients entered the double-blind treatment period and received study drug (mAb316P or placebo). Subsequently, patients returned to the clinic q2w for 22 weeks until day 155 for efficacy, safety, and laboratory assessments.

All patients received study drug (mAb316P or placebo) on days 15, 29, 43, 57, 71, 85, and day 99. Patients returned to the clinic for follow-up assessments on days 113, 127, 141, and 155. Patients were instructed to maintain a stable exercise level for the duration of the study, and to avoid rigorous exercise 48 hours before each study visit. Patients were queried on lipid lowering therapy duration and dosing at screening, and lipid lowering therapy compliance at every visit starting at visit 2/day −14.

During the double-blind treatment period, all patients received a total of 5 doses of mAb316P 150 mg SC and 4 doses of placebo SC as follows (FIG. 1): (i) All patients received an SC injection of placebo on day −14; (ii) On study day 1 (baseline), patients in group A received a dose of mAb316P, while patients in group B received a dose of placebo; (iii) On days 15, 29, and 43 patients in both groups (groups A and B) received mAb316P; (iv) On day 57, patients in group A received placebo, while patients in group B received mAb316P; (v) On day 71, patients in group A received mAb316P, while patients in group B received placebo; (vi) On day 85, patients in group A received placebo, while patients in group B received mAb316P; (vii) On day 99, patients in both group A and group B received placebo; and (viii) All patients returned for follow-up visits on days 113, 127, 141, and a visit on day 155.

Blood samples were collected for PK analyses at day 1 (baseline), and at every clinic visit starting at day 15. Blood samples were also collected for the analysis of anti-drug antibody levels at predetermined time points.

### Open Label Extension

All patients who successfully completed visit 15 (day 155) assessments were eligible to enter the open-label treatment period beginning on visit 16 (day 211) through visit 37 (end-of-study). During the open-label treatment period, all patients will receive an SC injection of mAb316P 150 mg q2w (±3 days) beginning on visit 17 (week 32) through visit 36 (end-of-treatment). Patients will be seen in the clinic at visit 16 (day 211), visit 17 (week 32), visit 18 (week 34), visit 19 (week 36), visit 20 (week 40), visit 21 (week 44), visit 22 (week 48), and then every 12 weeks from visit 23 (week 56) through visit 36 (end-of-treatment). Patients will return to the clinic 70 days after their last dose in the study for an end-of-study visit (visit 37). During the open-label treatment period, the patient will be trained to self-administer the mAb316P injections.

During the open-label treatment period, patients who achieve calculated LDL-C levels <25 mg/dL (0.65 mmol/L) on 2 consecutive laboratory assessments during the study will be monitored and managed as per a pre-established protocol. The investigator will be notified of 2 consecutive calculated LDL-C<25 mg/dL (0.65 mmol/L) levels. The efficacy of mAb316P in this population will be assessed by clinical laboratory evaluation of lipid levels. The safety of mAb316P in this population will be assessed by evaluating the incidence of adverse events (AEs) during the treatment-emergent periods, and by a detailed medical history, thorough physical examination, vital signs, weight, electrocardiogram (ECG), and clinical laboratory testing. Blinded safety data will be reviewed on an ongoing basis by the safety monitoring team (SMT). Concomitant medications will be collected from the time the patient signs the informed consent form (ICF) through visit 37 (end-of-study).

### Sample Analysis and Patient Procedures

Fasting blood samples (after an 8 to 12-hour fast) were collected at each clinic visit starting with the screening visit (visit 1 [day −28 to −15]). Laboratory tests performed in the lipid panel included measurement s of HDL-C, non-HDL-C, LDL-C (measured by ultracentrifugation and estimated by Freidwald equation), VLDL-C, total cholesterol, triglycerides, ApoA1, ApoB100, ApoB100/A1 ratio and Lp(a). On visit days when laboratory blood draws were obtained, study drug was administered after the blood draw.

In addition, serum samples for full blood chemistry testing were collected at the screening visit (visit 1 [day −28 to −15]), visit 2 (day −14), day 1 (baseline), days 15, 29, 43, 57, 71, 85, 99, 113, day 155, visit 16 (day 211), visit 17 (week 32), visit 18 (week 34), visit 19 (week 36), visit 21 (week 44), at study visit from visit 23 (week 56) to visit 35 (week 200), visit 36 (end-of-treatment), visit 37 (end-of-study) or early termination. Blood chemistry tests included: sodium, potassium chloride, calcium bicarbonate, glucose, creatinine, albumin, total protein, blood urea nitrogen, AST, ALT, alkaline phosphatase, lactate dehydrogenase, total bilirubin, total cholesterol, uric acid, CPK, and gamma glutamyl transpeptidase.

Blood samples for hematology testing were also collected at the screening visit (visit 1 [day −28 to −15]), day 1 (baseline), days 15, 29, 43, 57, 71, 85, 99, 113, 155, at visit 16 (day 211), visit 17 (week 32), visit 18 (week 34), visit 19 (week 36), visit 21 (week 44), at study visit from visit 23 (week 56) through visit 36 (end-of-treatment), and visit 37 (end-of-study) or early termination. Hematology tests included: hemoglobin, hematocrit, red blood cells, white blood cells, red cell indices, platelet count, and differential: neutrophils, lymphocytes, monocytes, basophils and eosinophils.

Blood samples for troponin I level assessment were also collected at all clinic visits, from the screening visit (visit 1 [day −28 to −15]) to day 155, except at visit 2 (day −14). In addition, urine samples for urinalysis were collected at the screening visit (visit 1 [day −28 to −15]), day 15, day 155, and at every study visit from visit 21 (week 44) through visit 36 (end-of-treatment), and visit 37 (end-of-study) or early termination visit. Urinalysis tests included: color, clarity, pH, specific gravity, ketones, protein, glucose, blood, bilirubin, leukocyte esterase, nitrate, white blood cells, red blood cells, hyaline and other casts, bacteria, epithelial cells, crystals, and yeast.

Other laboratory tests performed in the course of the study included pregnancy testing (for all women of childbearing potential), hs-CRP levels (collected on days −14, 1, 15, 43, 71, 99, 127, 155, visit 16 [day 211], and at every other visit from visit 22 [week 48] to visit 36 [end-of-treatment], visit 37 [end-of-study] or early termination visit), and PCSK9 levels (collected at every clinic visit starting with the screening visit [visit 1 {day −28 to −15}] through visit 15 [day 155]).

### Results

A total of 13 patients participated in the present study. Six patients were assigned to Group A (receiving mAb316P on days 1, 15, 29, 43 and 71, and placebo on days −14, 57 85 and 99) and seven patients were assigned to Group B (receiving mAb316P on days 15, 29, 43, 57 and 85, and placebo on days −14, 1, 71 and 99) (see FIG. 1). All patients completed the double-blind treatment period (to day 99). Seven patients completed the follow-up period (to day 155), 3 in Group A and 4 in Group B. Six patients agreed to participate in the open label treatment period, 2 in Group A and 4 in Group B. Demographics and baseline characteristics of the participating patients are shown in Table 2.

TABLE 2
Summary of Demographic and Baseline Characteristics

| | Group A | Group B | All Patients |
|---|---|---|---|
| | (n = 6) | (n = 7) | (n = 13) |
| Age | | | |
| Mean (SD) | 42.3 (14.72) | 46.6 (13.28) | 44.6 (13.54) |
| Median | 42.5 | 51.0 | 50.0 |
| Min:Max | 25:63 | 18:55 | 18:63 |
| Sex | | | |
| Male | 2 | 2 | 4 |
| Female | 4 | 5 | 9 |
| Race | | | |
| White | 5 | 6 | 11 |
| Indian Ocean Islander | 0 | 1 | 1 |
| Mauritius | 1 | 0 | 1 |
| Ethnicity | | | |
| Hispanic or Latino | 0 | 0 | 0 |
| Not Hispanic or Latino | 6 | 7 | 13 |
| Weight (kg) | | | |
| Mean (SD) | 82.8 (22.02) | 80.4 (16.05) | 81.5 (18.23) |
| Median | 80.9 | 81.8 | 81.8 |
| Min:Max | 55:118 | 60:108 | 55:118 |
| Height (cm) | | | |
| Mean (SD) | 170.1 (10.63) | 162.9 (3.34) | 166.2 (8.17) |
| Median | 169.7 | 163.0 | 165.0 |
| Min:Max | 158:183 | 156:166 | 156:183 |
| BMI (kg/m2) | | | |
| Mean (SD) | 28.5 (6.60) | 30.4 (6.74) | 29.5 (6.47) |
| Median | 26.0 | 31.1 | 26.8 |
| Min:Max | 22:38 | 22:41 | 22:41 |
| <30 | 4 | 3 | 7 |
| >=30 | 2 | 4 | 6 |
| Country | | | |
| FRA | 3 | 4 | 7 |
| USA | 3 | 3 | 6 |
| Lipid Lowering Therapy | | | |
| Statin | 6 | 7 | 13 |
| Ezetimibe | 3 | 3 | 6 |
| Niacin | 3 | 2 | 5 |
| Fibrate | 0 | 1 | 1 |
| Bile Acid Sequestrant | 0 | 1 | 1 |

A summary of baseline disease characteristics is shown in Table 3.

TABLE 3
Summary of Baseline Disease Characteristics

| | Group A | Group B | All Patients |
|---|---|---|---|
| | (n = 6) | (n = 7) | (n = 13) |
| Measured LDL-C | | | |
| (mg/dL) | | | |
| Mean (SD) | 108.83 (33.837) | 144.29 (68.390) | 127.92 (56.161) |
| Median | 93.5 | 136.0 | 111.0 |
| Min:Max | 78.0:168.0 | 82.0:284.0 | 78.0:284.0 |
| Calculated LDL-C | | | |
| (mg/dL) | | | |
| Mean (SD) | 101.50 (31.905) | 137.14 (66.739) | 120.69 (54.710) |
| Median | 86.00 | 124.00 | 101.00 |
| Min:Max | 71.0:154.0 | 73.0:272.0 | 71.0:272.0 |
| HDL-C (mg/dL) | | | |
| Mean (SD) | 57.17 (19.447) | 50.43 (14.741) | 53.54 (16.686) |
| Median | 57.50 | 53.00 | 55.00 |
| Min:Max | 25.0:84.0 | 22.0:70.0 | 22.0:84.0 |
| Non-HDL-C (mg/dL) | | | |
| Mean (SD) | 124.33 (48.997) | 165.86 (75.588) | 146.69 (65.736) |
| Median | 105.50 | 147.00 | 129.00 |
| Min:Max | 79.0:214.0 | 105.0:328.0 | 79.0:328.0 |
| VLDL-C (mg/dL) | | | |
| Mean (SD) | 22.83 (18.766) | 28.86 (14.983) | 26.08 (16.393) |
| Median | 17.00 | 29.00 | 22.00 |
| Min:Max | 9.0:60.0 | 11.0:56.0 | 9.0:60.0 |
| Apo B100 (mg/dL) | | | |
| Mean (SD) | 89.17 (27.287) | 101.00 (15.769) | 95.54 (21.732) |
| Median | 82.00 | 98.00 | 98.00 |
| Min:Max | 57.0:137.0 | 79.0:131.0 | 57.0:137.0 |
| Apo A1 (mg/dL) | | | |
| Mean (SD) | 136.33 (29.750) | 131.43 (30.021) | 133.69 (28.738) |
| Median | 139.5 | 139.00 | 139.00 |
| Min:Max | 90.0:175.0 | 70.0:156.0 | 70.0:175.0 |
| TG (mg/dL) | | | |
| Mean (SD) | 114.33 (94.449) | 144.29 (74.047) | 130.46 (81.853) |
| Median | 84.60 | 144.00 | 112.00 |
| Min:Max | 43.0:301.0 | 55.0:278.0 | 43.0:301.0 |
| Q1 | 61.0 | 66.0 | 66.0 |
| Q3 | 112.0 | 170.0 | 159.0 |
| Lp(a) (mg/dL) | | | |
| Mean (SD) | 53.60 (34.442) | 43.64 (61.891) | 48.24 (49.358) |
| Median | 56.55 | 19.40 | 33.40 |
| Min:Max | 2.0:103.0 | 2.0:178.0 | 2.0:178.0 |
| Q1 | 34.4 | 10.0 | 10.0 |
| Q3 | 69.1 | 56.1 | 66.6 |
| Screening HbA1c (%) | | | |
| Mean (SD) | 5.45 | 6.14 | 5.82 |
| Median | 5.35 | 6.00 | 5.70 |
| Min:Max | 5.1:6.1 | 5.5:7.2 | 5.1:7.2 |
| Glucose (mg/dL) | 97.7 ± 10.8 | 107.6 ± 17.8 | |
| Prior history of | 0 | 3 | 3 |
| diabetes mellitus | | | |
| Prior history of | 0 | 1 | 1 |
| glucose tolerance | | | |
| LDLR genotyping | 4 | 4 | 8 |
| performed prior to | | | |
| study | | | |
| Any LDLR variant | 0/4 | 0/4 | 0/8 |
| identified by history | | | |
| ApoB genotyping | 4 | 4 | 8 |
| performed prior to | | | |
| study | | | |
| Any ApoB variant | 0/4 | 0/4 | 0/8 |
| identified by history | | | |
| PCSK9 variant | | | |
| by history | | | |
| D374Y | 3 | 3 | 6 |
| L108R | 1 | 1 | 2 |
| R218S | 1 | 0 | 1 |
| S127R | 1 | 3 | 4 |
| PCSK9 variant by | | | |
| study-directed | | | |
| genotyping | | | |
| D374Y | 3 | 3 | 6 |
| L108R | 1 | 1 | 2 |
| R218S | 1 | 0 | 1 |
| S127R | 1 | 3 | 4 |

Tables 4-17 summarize the changes in cholesterol levels and other experimental parameters in the two treatment groups over the course of the study.

TABLE 4
Measured LDL-C at Day 15 in Conventional Unit : Analysis of Covariance

| | Group A | Group B |
|---|---|---|
| | (n = 6) | (n = 7) |
| Baseline assessment (mg/dL) | | |
| Mean (SD) | 108.83 (41.992) | 144.29 (43.235) |
| Median | 93.5 | 136.00 |
| Min:Max | 78.0:168.0 | 82.0:284.0 |
| Day 15 assessment (mg/dL) | | |
| Mean (SD) | 45.17 | 126.29 |
| Median | 30.00 | 133.00 |
| Min:Max | 14.0:128.0 | 63.0:196.0 |
| Day 15 percent change | | |
| from baseline | | |
| Mean (SD) | −61.90 (23.881) | −9.26 (12.813) |
| Median | −73.13 | −2.78 |
| Min:Max | −84.6:−23.7 | −30.9:3.0 |
| p-value | 0.0014** | 0.1043 |
| LS Mean (SE) | −62.48 (8.217) | −8.77 (7.575) |
| LS Mean Difference (SE) | −53.72 (11.486) | |
| 95% CI | (−79.31 to −28.12) | |
| p-value vs placebo | 0.0009*** | |
| Day 15 absolute change | | |
| from baseline | | |
| Mean (SD) | −63.67 (23.771) | −18.00 (31.937) |
| Median | −69.00 | −3.00 |
| Min:Max | −96.0:−32.0 | −88.0:3.0 |
| p-value | 0.0012** | 0.1865 |
| LS Mean (SE) | −70.08 (9.543) | −12.50 (8.797) |
| LS Mean Difference (SE) | −57.58 (13.340) | |
| 95% CI | (−87.30 to −27.85) | |
| p-value vs placebo | 0.0015** | |

TABLE 5
Proportions of Patients Achieving 30% Reduction from
Baseline in Measured LDL-C

| | Group A | | Group B | |
|---|---|---|---|---|
| | (n = 6) | | (n = 7) | |
| Day 15 | | | | |
| n % | 5 | (83.3%) | 1 | (14.3%) |
| p-value vs placebo | 0.0291* | | | |
| During the double-blind | | | | |
| treatment period | | | | |
| n % | 6 | (100.0%) | 7 | (100.0%) |
| Time to the first achievement of | | | | |
| 30% reduction in measured LDL-C | | | | |
| from first double blind IP (weeks) | | | | |
| Mean (SD) | 2.40 | (0.854) | 4.55 | (1.806) |

| | | |
|---|---|---|
| Median | 2.07 | 4.14 |
| Min:Max | 2.0:4.1 | 2.1:8.1 |

TABLE 6
ApoB100 at Day 15 in Conventional Unit: Analsis of Covariance

| | Group A | Group B |
|---|---|---|
| | (n = 6) | (n = 7) |
| Baseline assessment (mg/dL) | | |
| Mean (SD) | 89.17 (27.287) | 101.00 (15.769) |
| Median | 82.00 | 98.00 |
| Min:Max | 57.0:137.0 | 79.0:131.0 |
| Day 15 assessment (mg/dL) | | |
| Mean (SD) | 42.75 (43.404) | 99.43 (16.501) |
| Median | 26.25 | 102.00 |
| Min:Max | 17.5:129.0 | 67.0:123.0 |
| Day 15 percent change | | |
| from baseline | | |
| Mean (SD) | −55.86 (30.851) | −1.61 (7.969) |
| Median | −71.14 | −2.83 |
| Min:Max | −79.7:−5.8 | −15.2:9.7 |
| p-value | 0.0068** | 0.6122 |
| LS Mean (SE) | −53.33 (8.678) | −3.78 (8.008) |
| LS Mean Difference (SE) | −49.55 (12.050) | |
| 95% CI | (−76.39 to −22.70) | |
| p-value vs placebo | 0.0021** | |
| Day 15 absolute change | | |
| from baseline | | |
| Mean (SD) | −46.42 (26.622) | −1.57 (7.185) |
| Median | −60.00 | −3.00 |
| Min:Max | −68.5:−8.0 | −12.0:9.0 |
| p-value | 0.0079** | 0.5839 |
| LS Mean (SE) | −45.20 (7.996) | −2.61 (7.379) |
| LS Mean Difference (SE) | −42.59 (11.103) | |
| 95% CI | (−67.33 to −17.86) | |
| p-value vs placebo | 0.0033** | |

TABLE 7
Non-HDL-C at Day 15 in Conventional Unit: Analysis of Covariance

| | Group A | Group B |
|---|---|---|
| | (n = 6) | (n = 7) |
| Baseline assessment (mg/dL) | | |
| Mean (SD) | 124.33 (48.997) | 165.86 (75.588) |
| Median | 105.50 | 147.00 |
| Min:Max | 79.0:214.0 | 105.0:328.0 |
| Day 15 assessment (mg/dL) | | |
| Mean (SD) | 59.00 (58.100) | 149.00 (49.605) |
| Median | 40.00 | 150.00 |
| Min:Max | 20.0:176.0 | 80.0:243.0 |
| Day 15 percent change | | |
| from baseline | | |
| Mean (SD) | −56.93 (23.579) | −7.44 (13.073) |
| Median | −65.90 | 1.23 |
| Min:Max | −80.7:−17.7 | −25.9:3.6 |
| p-value | 0.0020** | 0.1826 |
| LS Mean (SE) | −56.87 (8.217) | −7.50 (7.575) |
| LS Mean Difference (SE) | −49.37 (11.487) | |
| 95% CI | (−74.96 to −23.77) | |
| p-value vs placebo | 0.0016** | |
| Day 15 absolute change | | |
| from baseline | | |
| Mean (SD) | −65.33 (27.090) | −16.86 (32.657) |
| Median | −69.00 | 2.00 |
| Min:Max | −102:−32.0 | −85.0:5.0 |
| p-value | 0.0020** | 0.2210 |
| LS Mean (SE) | −71.30 (10.961) | −11.74 (10.104) |
| LS Mean Difference (SE) | −59.56 (15.322) | |
| 95% CI | (−93.70 to −25.42) | |
| p-value vs placebo | 0.0030** | |

TABLE 8
Total Cholesterol at Day 15 in Conventional Unit: Analysis of Covariance

| | Group A | Group B |
|---|---|---|
| | (n = 6) | (n = 7) |
| Baseline assessment (mg/dL) | | |
| Mean (SD) | 181.33 (41.889) | 216.29 (78.612) |
| Median | 163.50 | 201.00 |
| Min:Max | 139.0:239.0 | 140.0:380.0 |
| Day 15 assessment (mg/dL) | | |
| Mean (SD) | 117.67 (44.437) | 196.00 (50.777) |
| Median | 103.00 | 198.00 |
| Min:Max | 76.0:198.0 | 136.0:287.0 |
| Day 15 percent change | | |
| from baseline | | |
| Mean (SD) | −35.81 (14.546) | −7.14 (10.463) |
| Median | −42.29 | −1.73 |
| Min:Max | −50.8:−17.2 | −24.5:3.0 |
| p-value | 0.0018** | 0.1210 |
| LS Mean (SE) | −36.94 (5.203) | −6.18 (4.802) |
| LS Mean Difference (SE) | −30.75 (7.224) | |
| 95% CI | (−46.85 to −14.66) | |
| p-value vs placebo | 0.0017** | |
| Day 15 absolute change | | |
| from baseline | | |
| Mean (SD) | −63.67 (25.594) | −20.29 (34.717) |
| Median | −63.67 (25.594) | −3.00 |
| Min:Max | −94.0:−25.0 | −93.0:5.0 |
| p-value | 0.0017** | 0.1731 |
| LS Mean (SE) | −70.03 (9.579) | −14.83 (8.840) |
| LS Mean Difference (SE) | −55.20 (13.300) | |
| 95% CI | (−84.84 to −25.57) | |
| p-value vs placebo | 0.0020** | |

TABLE 9
Ratio of ApoB100/ApoA1 at Day 15: Analysis of Covariance

| | Group A | Group B |
|---|---|---|
| | (n = 6) | (n = 7) |
| Baseline assessment | | |
| Mean (SD) | 0.71 (0.401) | 0.82 (0.296) |
| Median | 0.57 | 0.81 |
| Min:Max | 0.5:1.5 | 0.5:1.4 |
| Day 15 assessment | | |
| Mean (SD) | 0.37 (0.504) | 0.80 (0.302) |
| Median | 0.16 | 0.83 |
| Min:Max | 0.1:1.4 | 0.4:1.4 |
| Day 15 absolute change | | |
| from baseline | | |
| Mean (SD) | −0.34 (0.152) | −0.03 (0.048) |
| Median | −0.38 | −0.02 |
| Min:Max | −0.5:−0.1 | −0.1:0.0 |
| p-value | 0.0029** | 0.2082 |
| LS Mean (SE) | −0.33 (0.042) | −0.03 (0.039) |
| LS Mean Difference (SE) | −0.30 (0.058) | |
| 95% CI | (−0.42 to −0.17) | |
| p-value vs placebo | 0.0005*** | |

TABLE 10
Calculated LDL-C at Day 15 in Conventional Unit: Analysis of Covariance

| | Group A | Group B |
|---|---|---|
| | (n = 6) | (n = 7) |
| Baseline assessment (mg/dL) | | |
| Mean (SD) | 101.50 (31.905) | 137.14 (66.739) |
| Median | 86.00 | 124.00 |
| Min:Max | 71.0:154.0 | 73.0:272.0 |
| Day 15 assessment (mg/dL) | | |
| Mean (SD) | 39.83 (38.175) | 119.71 (47.853) |
| Median | 29.00 | 116.00 |
| Min:Max | 11.0:115.0 | 57.0:205.0 |
| Day 15 percent change | | |
| from baseline | | |
| Mean (SD) | −63.82 (23.896) | −10.72 (9.863) |
| Median | −71.90 | −6.56 |
| Min:Max | −87.0:−25.4 | −24.6:1.4 |
| p-value | 0.0012** | 0.0283* |
| LS Mean (SE) | −63.82 (7.829) | −10.71 (7.215) |
| LS Mean Difference (SE) | −53.11 (10.962) | |
| 95% CI | (−77.53 to −28.68) | |
| p-value vs placebo | 0.0007*** | |
| Day 15 absolute change | | |
| from baseline | | |
| Mean (SD) | −61.67 (24.130) | −17.43 (23.358) |
| Median | −63.50 | −8.00 |
| Min:Max | −97.0:−32.0 | −67.0:2.0 |
| p-value | 0.0015** | 0.0958 |
| LS Mean (SE) | −66.79 (8.360) | −13.04 (7.704) |
| LS Mean Difference (SE) | −53.75 (11.706) | |
| 95% CI | (−79.83 to −27.66) | |
| p-value vs placebo | 0.0010*** | |

TABLE 11
HDL-C at Day 15 in Conventional Unit: Analysis of Covariance

| | Group A | Group B |
|---|---|---|
| | (n = 6) | (n = 7) |
| Baseline assessment (mg/dL) | | |
| Mean (SD) | 57.17 (19.447) | 50.43 (14.741) |
| Median | 57.50 | 53.00 |
| Min:Max | 25.0:84.0 | 22.0:70.0 |
| Day 15 assessment (mg/dL) | | |
| Mean (SD) | 58.67 (22.624) | 47.00 (15.144) |
| Median | 58.50 | 47.00 |
| Min:Max | 22.0:92.0 | 23.0:72.0 |
| Day 15 percent change | | |
| from baseline | | |
| Mean (SD) | 1.40 (13.481) | −6.45 (7.948) |
| Median | 8.46 | −10.95 |
| Min:Max | −19.1:11.6 | −14.9:3.4 |
| p-value | 0.8088 | 0.0754 |
| LS Mean (SE) | 1.04 (4.631) | −6.14 (4.280) |
| LS Mean Difference (SE) | 7.18 (6.376) | |
| 95% CI | (−7.03 to 21.39) | |
| p-value vs placebo | 0.2864 | |
| Day 15 absolute change | | |
| from baseline | | |
| Mean (SD) | 1.50 (8.118) | −3.43 (4.237) |
| Median | 5.00 | −6.00 |
| Min:Max | −13.0:8.0 | −8.0:2.0 |
| p-value | 0.6698 | 0.0761 |
| LS Mean (SE) | 1.33 (2.710) | −3.28 (2.505) |
| LS Mean Difference (SE) | 4.61 (3.731) | |
| 95% CI | (−3.70 to 12.92) | |
| p-value vs placebo | 0.2450 | |

TABLE 12
VLDL-C at Day 15 in Conventional Unit: Analysis of Covariance

| | Group A | Group B |
|---|---|---|
| | (n = 6) | (n = 7) |
| Baseline assessment (mg/dL) | | |
| Mean (SD) | 22.83 (18.766) | 28.86 (14.983) |
| Median | 17.00 | 29.00 |
| Min:Max | 9.0:60.0 | 11.0:56.0 |
| Day 15 assessment (mg/dL) | | |
| Mean (SD) | 19.17 (20.721) | 29.29 (12.919) |
| Median | 11.50 | 34.00 |
| Min:Max | 8.0:61.0 | 12.0:44.0 |
| Day 15 percent change | | |
| from baseline | | |
| Mean (SD) | −23.67 (17.608) | 6.56 (27.208) |
| Median | −28.09 | 10.71 |
| Min:Max | −46.3:1.9 | −32.4:37.3 |
| p-value | 0.0216* | 0.5474 |
| LS Mean (SE) | −23.81 (10.089) | 6.68 (9.328) |
| LS Mean Difference (SE) | −30.49 (13.866) | |
| 95% CI | (−61.38 to 0.41) | |
| p-value vs placebo | 0.0526 | |
| Day 15 absolute change | | |
| from baseline | | |
| Mean (SD) | −3.67 (3.204) | 0.43 (10.612) |
| Median | −4.00 | 1.00 |
| Min:Max | −7.0:1.0 | −18.0:11.0 |
| p-value | 0.0379* | 0.9184 |
| LS Mean (SE) | −4.02 (3.423) | 0.74 (3.165) |
| LS Mean Difference (SE) | −4.76 (4.705) | |
| 95% CI | (−15.24 to 5.72) | |
| p-value vs placebo | 0.3355 | |

TABLE 13
ApoA1 at Day 15 in Conventional Unit: Analysis of Covariance

| | Group A | Group B |
|---|---|---|
| | (n = 6) | (n = 7) |
| Baseline assessment (mg/dL) | | |
| Mean (SD) | 136.33 (29.750) | 131.43 (30.021) |
| Median | 139.50 | 139.00 |
| Min:Max | 90.0:175.0 | 70.0:156.0 |
| Day 15 assessment (mg/dL) | | |
| Mean (SD) | 141.17 (30.954) | 134.57 (34.278) |
| Median | 138.00 | 143.00 |
| Min:Max | 93.0:181.0 | 74.0:185.0 |
| Day 15 percent change | | |
| from baseline | | |
| Mean (SD) | 3.96 (9.820) | 2.40 (9.099) |
| Median | 5.81 | 0.00 |
| Min:Max | −14.5:13.4 | −5.6:20.9 |
| p-value | 0.3690 | 0.5118 |
| LS Mean (SE) | 4.05 (4.023) | 2.32 (3.724) |
| LS Mean Difference (SE) | 1.73 (5.493) | |
| 95% CI | (−10.51 to 13.97) | |
| p-value vs placebo | 0.7593 | |
| Day 15 absolute change | | |
| from baseline | | |
| Mean (SD) | 4.83 (14.162) | 3.14 (13.545) |
| Median | 8.00 | 0.00 |
| Min:Max | −22.0:17.0 | −7.0:32.0 |
| p-value | 0.4413 | 0.5618 |
| LS Mean (SE) | 4.85 (5.934) | 3.13 (5.492) |
| LS Mean Difference (SE) | 1.71 (8.101) | |
| 95% CI | (−16.34 to 19.76) | |
| p-value vs placebo | 0.8366 | |

TABLE 14
TG at Day 15 in Conventional Unit: Rank-Based Analysis of Covariance

| | Group A | Group B |
|---|---|---|
| | (n = 6) | (n = 7) |
| Baseline assessment (mg/dL) | | |
| Median | 84.50 | 144.00 |
| Mean (SD) | 114.33 (94.449) | 144.29 (74.047) |
| Q1:Q3 | 61.00:112.00 | 66.00:170.00 |
| Min:Max | 43.0:301.0 | 55.0:278.0 |
| Day 15 assessment (mg/dL) | | |
| Median | 55.00 | 167.00 |
| Mean (SD) | 95.83 (104.519) | 146.71 (63.560) |
| Q1:Q3 | 41.00:76.00 | 72.00:199.00 |
| Min:Max | 41.0:307.0 | 62.0:221.0 |
| Day 15 percent change | | |
| from baseline | | |
| Median | −27.87 | 12.90 |
| Mean (SD) | −23.12 (17.921) | 7.29 (26.985) |
| Q1:Q3 | −33.33:−6.12 | −27.22:29.69 |
| Min:Max | −45.6:2.1 | −31.5:38.0 |
| p-value | 0.0625 | 0.5781 |
| Difference in median vs placebo | −40.77 | |
| p-value vs placebo | 0.0461* | |
| Day 15 absolute change | | |
| from baseline | | |
| Median | −21.00 | 7.00 |
| Mean (SD) | −18.50 (17.524) | 2.43 (51.807) |
| Q1:Q3 | −36.00:−2.00 | −43.00:51.00 |
| Min:Max | −37.0:6.0 | −88.0:55.0 |
| p-value | 0.0938 | 0.6875 |
| Difference in median vs placebo | −28.00 | |
| p-value vs placebo | 0.2125 | |

TABLE 15
Lp(a) at Day 15 in Conventional Unit: Rank-Based Analysis of Covariance

| | Group A | Group B |
|---|---|---|
| | (n = 6) | (n = 7) |
| Baseline assessment (mg/dL) | | |
| Median | 56.55 | 19.40 |
| Mean (SD) | 53.60 (34.442) | 43.64 (61.891) |
| Q1:Q3 | 34.40:69.10 | 9.95:56.10 |
| Min:Max | 2.0:103.0 | 2.0:178.0 |
| Day 15 assessment (mg/dL) | | |
| Median | 37.15 | 13.00 |
| Mean (SD) | 42.13 (33.902) | 47.04 (73.443) |
| Q1:Q3 | 16.60:77.40 | 8.95:57.60 |
| Min:Max | 2.0:82.5 | 2.0:208.0 |
| Day 15 percent change | | |
| from baseline | | |
| Median | −20.95 | 0.00 |
| Mean (SD) | −21.78 (28.592) | −4.26 (15.382) |
| Q1:Q3 | −40.70:0.00 | −10.05:2.67 |
| Min:Max | −64.3:16.2 | −33.0:16.9 |
| p-value | 0.1250 | 0.6875 |
| Difference in median vs placebo | −20.95 | |
| p-value vs placebo | 0.1317 | |
| Day 15 absolute change | | |
| from baseline | | |
| Median | −14.60 | 0.00 |
| Mean (SD) | −11.47 (14.622) | 3.40 (12.001) |
| Q1:Q3 | −20.50:0.00 | −1.00:1.50 |
| Min:Max | −29.9:10.8 | −6.4:30.0 |
| p-value | 0.1250 | 1.0000 |
| Difference in median vs placebo | −14.60 | |
| p-value vs placebo | 0.0999 | |

In the foregoing tables (Table 3-14), [1] indicates that p-value was obtained through one sample t-test. *, ** and *** P-value are significant at 0.05, 0.01 and 0.001 level, respectively.

Changes in LDL-C levels over the course of the study are summarized in Table 16.

TABLE 16
Changes in LDL-C Levels From Baseline at Various Time Points
[ Values Expressed as Mean LDL-C mg/mL (SD) ]

| | Group A | | Group B | | All Patients | |
|---|---|---|---|---|---|---|
| | (n = 6) | | (n = 7) | | (n = 13) | |
| Baseline | 108.8 | (33.84) | 125.9 | (43.64) | 118.0 | (38.83) |
| Week 2 | 45.2 | (41.99) | 60.4 | (28.54) | 53.4 | (34.71) |
| Change from Baseline | −63.7 | (23.77) | −65.4 | (38.21) | −64.6 | (31.08) |
| Percent Change from | −61.9 | (23.88) | −49.9 | (27.96) | −55.5 | (25.83) |
| Baseline | | | | | | |
| Week 4 | 41.8 | (37.38) | 54.7 | (34.10) | 48.8 | (34.76) |
| Change from Baseline | −67.0 | (21.65) | −71.1 | (37.16) | −69.2 | (29.84) |
| Percent Change from | −64.5 | (21.80) | −55.9 | (26.57) | −59.9 | (23.89) |
| Baseline | | | | | | |
| Week 6 | 32.2 | (15.69) | 41.9 | (17.84) | 37.4 | (16.94) |
| Change from Baseline | −76.7 | (27.84) | −84.0 | (41.67) | −80.6 | (34.72) |
| Percent Change from | −69.9 | (14.53) | −63.3 | (21.22) | −66.4 | (18.02) |
| Baseline | | | | | | |
| Week 8 | 31.3 | (14.72) | 33.1 | (27.09) | 32.3 | (21.40) |
| Change from Baseline | −77.5 | (34.86) | −92.7 | (24.42) | −85.7 | (29.44) |
| Percent Change from | −69.5 | (18.01) | −76.5 | (14.85) | −73.3 | (16.08) |
| Baseline | | | | | | |
| Week 10 | 55.8 | (26.84) | 76.1 | (40.68) | 66.8 | (35.20) |
| Change from Baseline | −53.0 | (34.44) | −49.7 | (51.24) | −51.2 | (42.54) |
| Percent Change from | −47.5 | (27.38) | −32.1 | (42.40) | −39.2 | (35.71) |
| Baseline | | | | | | |
| Week 12 | 41.2 | (24.59) | 49.9 | (19.92) | 45.8 | (21.69) |
| Change from Baseline | −67.7 | (35.99) | −76.0 | (43.67) | −72.2 | (38.88) |
| Percent Change from | −61.0 | (26.41) | −56.2 | (21.53) | −58.4 | (22.99) |
| Baseline | | | | | | |
| Week 14 | 81.0 | (37.23) | 54.2 | (23.22) | 68.8 | (33.24) |
| Change from Baseline | −27.8 | (35.19) | −61.2 | (56.00) | −43.0 | (46.66) |
| Percent Change from | −25.2 | (30.70) | −43.6 | (39.28) | −33.6 | (34.36) |
| Baseline | | | | | | |
| Week 16 | 104.8 | (43.14) | 99.1 | (49.50) | 101.5 | (44.96) |
| Change from Baseline | −7.2 | (18.71) | −26.7 | (39.92) | −18.6 | (33.13) |
| Percent Change from | −8.2 | (20.21) | −18.5 | (27.93) | −14.2 | (24.53) |
| Baseline | | | | | | |
| Week 18 | 118.8 | (63.28) | 97.4 | (40.13) | 107.3 | (50.96) |
| Change from Baseline | 10.0 | (30.61) | −28.4 | (41.39) | −10.7 | (40.55) |
| Percent Change from | 3.9 | (23.96) | −16.9 | (33.08) | −7.3 | (30.04) |
| Baseline | | | | | | |
| Week 20 | 108.7 | (60.67) | 109.9 | (49.34) | 109.3 | (52.45) |
| Change from Baseline | −0.2 | (31.42) | −16.0 | (36.83) | −8.7 | (34.02) |
| Percent Change from | −5.3 | (29.42) | −9.9 | (26.27) | −7.8 | (26.67) |
| Baseline | | | | | | |

| | | | | |
|---|---|---|---|---|
| Week 22 | 106.7 | (63.56) | N/A | N/A |
| Change from Baseline | −2.2 | (33.01) | N/A | N/A |
| Percent Change from | −7.5 | (29.13) | N/A | N/A |
| Baseline | | | | |

TABLE 17
Lipid Parameters in Group A and B Patients Combined After 8 Weeks of mAb316P Treatment

| | | | 8 Weeks of mAb316P |
|---|---|---|---|
| | Baseline | | Treatment |
| | Group A (N = 6) | Group B (N = 7) | Combined (p-value) |
| LDL-C (measured, mg/dL) | 108.8 ± 33.8 | 144.3 ± 68.4 | 32.3 ± 21.4 |
| % change from baseline | | | −73.3 ± 16.1 (<0.0001) |
| HDL-C (mg/dL) | 57.2 ± 19.4 | 50.4 ± 14.7 | 55.8 ± 18.0 |
| % change from baseline | | | 7.9 ± 13.7 (0.0603) |
| Triglycerides (mg/dL, | 84.5 (61.0:112.0) | 144.0 (66.0:170.0) | 64.0 (42:86) |
| median [IQR] | | | |
| % change from baseline | | | −37.8 (−46:−27) |
| | | | (0.0002) |
| VLDL-C (measured, mg/dL) | 22.8 ± 18.8 | 28.9 ± 15.0 | 14.4 ± 8.1 |
| % change from baseline | | | −39.5 ± 17.5 (<0.0001) |
| ApoB-100 (mg/dL) | 89.2 ± 27.3 | 101.0 ± 15.8 | 32.4 ± 15.3 |
| % change from baseline | | | −65.0 ± 16.6 (<0.0001) |
| Lp(a) (mg/dL, median [IQR]) | 56.6 (34.4:69.1) | 19.4 (10.0:56.1) | 11.9 (4:51) |
| % change from baseline | | | −43.3 (−65:4) |
| | | | (0.0020) |

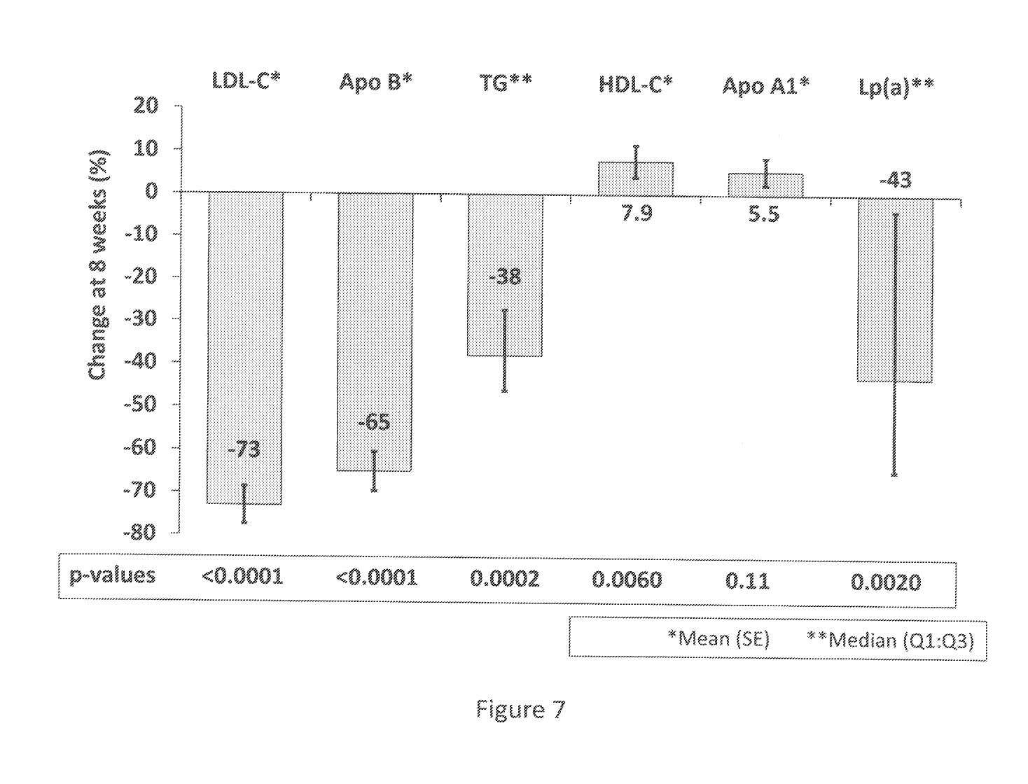

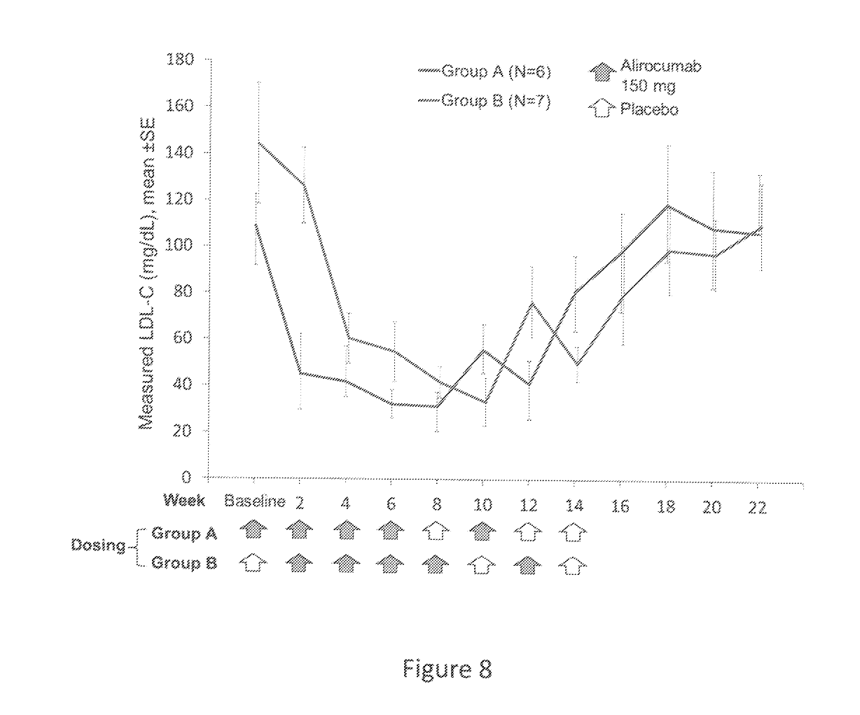

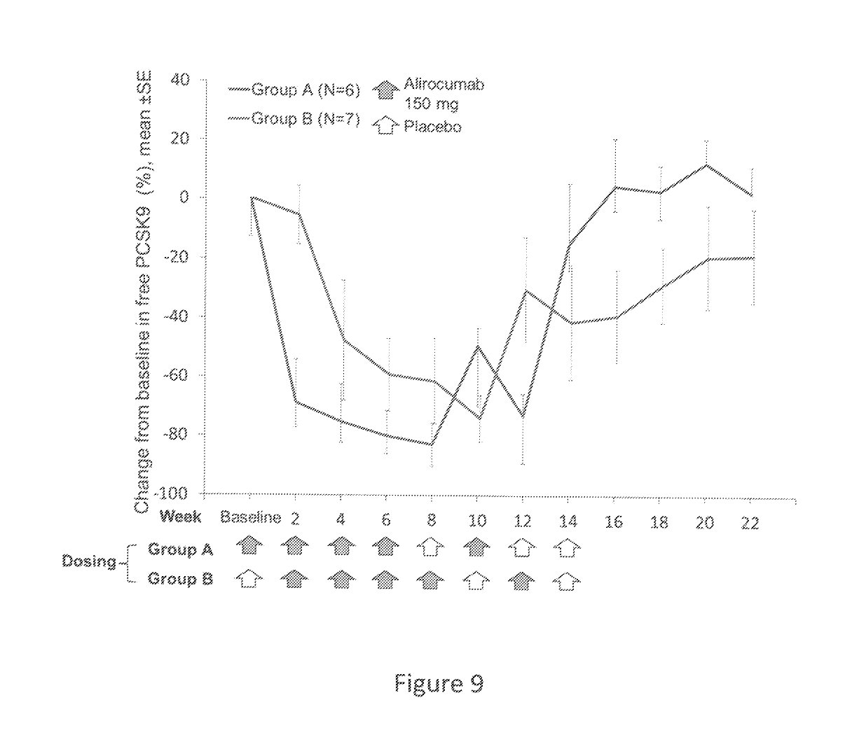

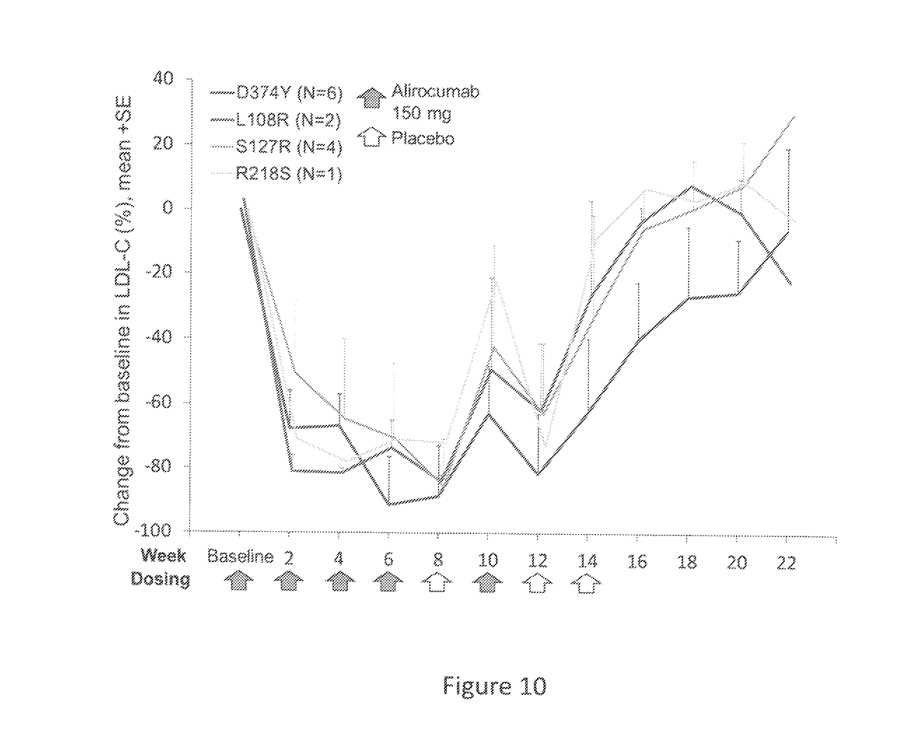

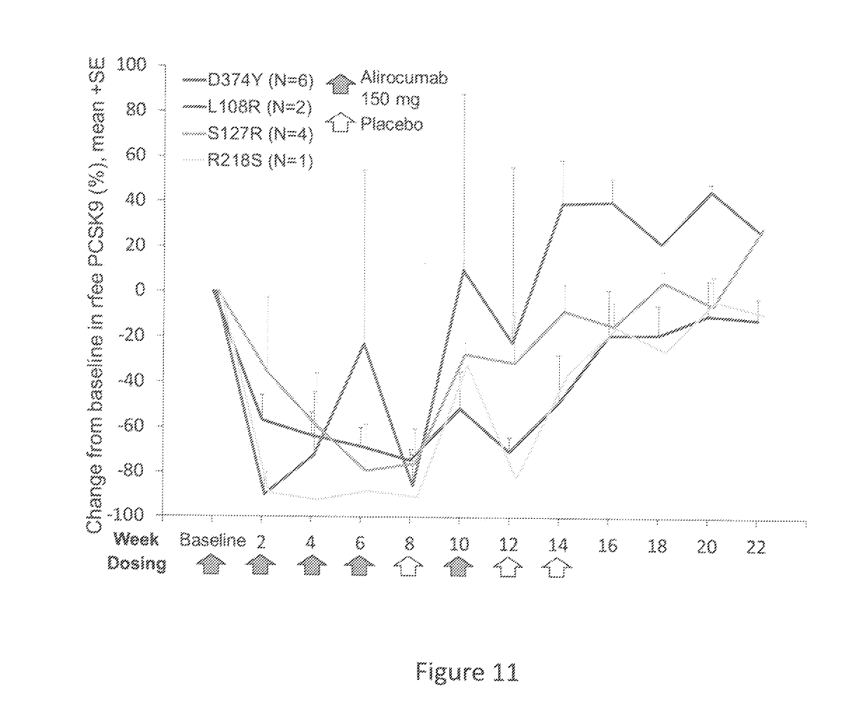

The percent changes from baseline in lipid levels (LDL-C, ApoB, TG, HDL-C, ApoA1 and Lp(a)) at week 8 for all 13 patients are summarized in FIG. 7. Changes in LDL-C and free PCSK9 levels for the different treatment groups and various GOFm populations are shown in FIGS. 8-11.

### Summary/Conclusions

Patients who enrolled in this study (mean age 44.6 years, mean LDL-C at baseline 127.9 mg/dL) carried four different PCSK9 GOF mutations (L108R, S127R, R218S, and D374Y). All patients completed the double-blind (to week 14) and safety follow-up (to week 22) parts of the study.

In the present study, mAb316P administration significantly reduced LDL cholesterol in all patients with PCSK9 GOFm enrolled, and this was temporally correlated with free PCSK9 reductions. By utilizing a novel randomized placebo-phase design, each patient contributed to the safety and efficacy data, while still enabling comparison of mAb316P administration to placebo. During the 2-week placebo-controlled portion of the trial, mAb316P administration also significantly reduced apolipoprotein B and triglycerides. In particular, at week 2, LS mean reductions from baseline (mAb316P vs placebo) were: LDL-C 53.7%, ApoB 49.6%, non-HDL-C 49.4%, total cholesterol 30.7%, and ApoB/ApoA1 0.30 (all p-values 5 0.002). A post-hoc pooled analysis of all subjects after 8 weeks of alirocumab treatment also revealed statistically significant reductions of Lp(a) and very low density cholesterol. In addition, treatment was generally well tolerated.

While all mutation carriers responded to treatment, our results suggest that the rate of reduction in LDL cholesterol may differ in patients carrying different GOF mutations, and this may correlate with the rate reduction of free PCSK9 after alirocumab administration.

This study confirms that mAb316P is a promising therapeutic option for patients with autosomal dominant hypercholesterolemia caused by PCSK9 gain-of-function mutations.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

What is claimed is:

1. A method for treating autosomal dominant hypercholesterolemia (ADH), wherein the method comprises selecting a patient who carries a gain-of-function mutation (GOFm) in one or both alleles of the PCSK9 gene, and administering to the patient a pharmaceutical composition comprising a PCSK9 inhibitor.

2. The method of claim 1, wherein the GOFm encodes a PCSK9 variant protein comprising an amino acid substitution selected from the group consisting of V4I, E32K, D35Y, E48K, P71L, R96C, L108R, S127R, D129N, R215H, F216L, R218S, R357H, D374H, D374Y, S465L and R496W.

3. The method of claim 1, wherein the GOFm encodes a PCSK9 variant protein comprising an amino acid substitution selected from the group consisting of S127R, F216L, R218S, R357H and D374Y.

4. The method of claim 1, wherein the patient is further selected on the basis of having a plasma LDL-C level greater than or equal to about 70 mg/dL prior to administration of the pharmaceutical composition comprising the PCSK9 inhibitor.

5. The method of claim 1, wherein the patient is on a background lipid lowering therapy at the time of and/or prior to administration of the pharmaceutical composition comprising the PCSK9 inhibitor.

6. The method of claim 5, wherein the background lipid lowering therapy is selected from the group consisting of statins, ezetimibe, fibrates, niacin, omega-3 fatty acids, and bile acid resins.

7. The method of claim 1, wherein the patient is further selected on the basis of having a body mass index (BMI) of greater than about 18.0 kg/m2 prior to administration of the pharmaceutical composition comprising the PCSK9 inhibitor.

8. The method of claim 1, wherein the PCSK9 inhibitor is an antibody or antigen-binding fragment thereof that specifically binds PCSK9.

9. The method of claim 8, wherein the pharmaceutical composition comprises 20 mg to 200 mg of the PCSK9 inhibitor.

10. The method of claim 9, wherein the pharmaceutical composition comprises 50 mg to 150 mg of the PCSK9 inhibitor.

11. The method of claim 10, wherein the pharmaceutical composition comprises 50 mg of the PCSK9 inhibitor.

12. The method of claim 10, wherein the pharmaceutical composition comprises 75 mg of the PCSK9 inhibitor.

13. The method of claim 10, wherein the pharmaceutical composition comprises 100 mg of the PCSK9 inhibitor.

14. The method of claim 10, wherein the pharmaceutical composition comprises 150 mg of the PCSK9 inhibitor.

15. The method of claim 8, wherein the antibody or antigen binding fragment thereof comprises the heavy and light chain CDRs of a HCVR/LCVR amino acid sequence pair selected from the group consisting of SEQ ID NOs: 90/92 and 218/226.

16. The method of claim 15, wherein the antibody or antigen-binding fragment thereof comprises heavy and light chain CDR amino acid sequences having SEQ ID NOs:220, 222, 224, 228, 230 and 232.

17. The method of claim 16, wherein the antibody or antigen-binding fragment thereof comprises an HCVR having the amino acid sequence of SEQ ID NO:218 and an LCVR having the amino acid sequence of SEQ ID NO:226.

18. The method of claim 15, wherein the antibody or antigen-binding fragment thereof comprises heavy and light chain CDR amino acid sequences having SEQ ID NOs:76, 78, 80, 84, 86 and 88.

19. The method of claim 18, wherein the antibody or antigen-binding fragment thereof comprises an HCVR having the amino acid sequence of SEQ ID NO:90 and an LCVR having the amino acid sequence of SEQ ID NO:92.

20. The method of claim 8, wherein the antibody or antigen-binding fragment thereof binds to the same epitope on PCSK9 as an antibody comprising heavy and light chain CDR amino acid sequences having SEQ ID NOs:220, 222, 224, 228, 230 and 232; or SEQ ID NOs: 76, 78, 80, 84, 86 and 88.

21. The method of claim 8, wherein the antibody or antigen-binding fragment thereof competes for binding to PCSK9 with an antibody comprising heavy and light chain CDR amino acid sequences having SEQ ID NOs:220, 222, 224, 228, 230 and 232; or SEQ ID NOs: 76, 78, 80, 84, 86 and 88.

22. The method claim 8, wherein the antibody or antigen binding fragment thereof exhibits pH-dependent binding characteristics to PCSK9.

23. The method of claim 22, wherein the antibody or antigen-binding fragment thereof binds PCSK9 at neutral pH with a higher affinity than at acidic pH.

24. A therapeutic regimen for treating autosomal dominant hypercholesterolemia (ADH), wherein the therapeutic regimen comprises selecting a patient who carries a gain-of-function mutation (GOFm) in one or both alleles of the PCSK9 gene, and administering to the patient a plurality of doses of a pharmaceutical composition comprising a PCSK9 inhibitor, wherein the doses are administered to the patient at a dosing frequency selected from the group consisting of: once a week, once every two weeks, once every three weeks, once every four weeks, once every six weeks, once every eight weeks, once every ten weeks and once every twelve weeks.

25. The method of claim 24, wherein the GOFm encodes a PCSK9 variant protein comprising an amino acid substitution selected from the group consisting of V4I, E32K, D35Y, E48K, P71L, R96C, L108R, S127R, D129N, R215H, F216L, R218S, R357H, D374H, D374Y, S465L and R496W.

26. The method of claim 24, wherein the GOFm encodes a PCSK9 variant protein comprising an amino acid substitution selected from the group consisting of S127R, F216L, R218S, R357H and D374Y.

27. The method of claim 24, wherein the patient is further selected on the basis of having a plasma LDL-C level greater than or equal to about 70 mg/dL prior to administration of the pharmaceutical composition comprising the PCSK9 inhibitor.

28. The method of claim 24, wherein the patient is on a background lipid lowering therapy at the time of and/or prior to the first administration of the pharmaceutical composition comprising the PCSK9 inhibitor.

29. The method of claim 28, wherein the background lipid lowering therapy is selected from the group consisting of statins, ezetimibe, fibrates, niacin, omega-3 fatty acids, and bile acid resins.

30. The method of claim 24, wherein the patient is further selected on the basis of having a body mass index (BMI) of greater than about 18.0 kg/m2 prior to the first administration of the pharmaceutical composition comprising the PCSK9 inhibitor.

31. The method of claim 24, wherein the PCSK9 inhibitor is an antibody or antigen-binding fragment thereof that specifically binds PCSK9.

32. The method of claim 31, wherein the pharmaceutical composition comprises 20 mg to 200 mg of the PCSK9 inhibitor.

33. The method of claim 32, wherein the pharmaceutical composition comprises 50 mg to 150 mg of the PCSK9 inhibitor.

34. The method of claim 33, wherein the pharmaceutical composition comprises 50 mg of the PCSK9 inhibitor.

35. The method of claim 33, wherein the pharmaceutical composition comprises 75 mg of the PCSK9 inhibitor.

36. The method of claim 33, wherein the pharmaceutical composition comprises 100 mg of the PCSK9 inhibitor.

37. The method of claim 33, wherein the pharmaceutical composition comprises 150 mg of the PCSK9 inhibitor.

38. The method of claim 31, wherein the antibody or antigen binding fragment thereof comprises the heavy and light chain CDRs of a HCVR/LCVR amino acid sequence pair selected from the group consisting of SEQ ID NOs: 90/92 and 218/226.

39. The method of claim 38, wherein the antibody or antigen-binding fragment thereof comprises heavy and light chain CDR amino acid sequences having SEQ ID NOs:220, 222, 224, 228, 230 and 232.

40. The method of claim 39, wherein the antibody or antigen-binding fragment thereof comprises an HCVR having the amino acid sequence of SEQ ID NO:218 and an LCVR having the amino acid sequence of SEQ ID NO:226.

41. The method of claim 38, wherein the antibody or antigen-binding fragment thereof comprises heavy and light chain CDR amino acid sequences having SEQ ID NOs:76, 78, 80, 84, 86 and 88.

42. The method of claim 41, wherein the antibody or antigen-binding fragment thereof comprises an HCVR having the amino acid sequence of SEQ ID NO:90 and an LCVR having the amino acid sequence of SEQ ID NO:92.

43. The method of claim 31, wherein the antibody or antigen-binding fragment thereof binds to the same epitope on PCSK9 as an antibody comprising heavy and light chain CDR amino acid sequences having SEQ ID NOs:220, 222, 224, 228, 230 and 232; or SEQ ID NOs: 76, 78, 80, 84, 86 and 88.

44. The method of claim 31, wherein the antibody or antigen-binding fragment thereof competes for binding to PCSK9 with an antibody comprising heavy and light chain CDR amino acid sequences having SEQ ID NOs:220, 222, 224, 228, 230 and 232; or SEQ ID NOs: 76, 78, 80, 84, 86 and 88.

45. The method of claim 31, wherein the antibody or antigen binding fragment thereof exhibits pH-dependent binding characteristics to PCSK9.

46. The method of claim 45, wherein the antibody or antigen-binding fragment thereof binds PCSK9 at neutral pH with a higher affinity than at acidic pH.

47. A cascade screening method to identify subjects having, or at risk of developing autosomal dominant hypercholesterolemia (ADH), the method comprising: (a) identifying a first individual with ADH who carries a particular PCSK9 gain-of-function mutation (“PCSK9-GOFm”); and (b) screening biologically-related family members of the first individual for the presence of the PCSK9-GOFm; wherein the presence of the PCSK9-GOFm in one or more of biologically related family members identifies such family members as subjects having, or at risk of developing ADH.

48. The method of claim 47, wherein the PCSK9-GOFm is selected from the group consisting of V4I, E32K, D35Y, E48K, P71L, R96C, L108R, S127R, D129N, R215H, F216L, R218S, R357H, D374H, D374Y, S465L and R496W.
